(19) United States
(12) Patent Application Publication
(10) Pub. No.: US 2025/0242080 A1
(43) Pub. Date: Jul. 31, 2025

(54) NOVEL METHOD TO ALTER THE PROTEOME OF A DISEASED HEART BY INJECTION OF ECM PARTICLES PRODUCED FROM DECELLULARIZED 3D MICROTISSUES OF HUMAN MESENCHYMAL STEM CELLS

(71) Applicants: Brown University, Providence, RI (US); XM Therapeutics, Inc., Providence, RI (US)

(72) Inventors: Jeffrey R. Morgan, Sharon, MA (US); Frank Ahmann, Laguna Niguel, CA (US); Max Johan Petersen, Providence, RI (US)

(21) Appl. No.: 19/067,759

(22) Filed: Feb. 28, 2025

Related U.S. Application Data
(63) Continuation-in-part of application No. 18/339,980, filed on Jun. 22, 2023.
Application No. 18/339,980 is a continuation of application No. PCT/US2021/064834, filed on Dec. 22, 2021.
(60) Provisional application No. 63/560,539, filed on Mar. 1, 2024.
(60) Provisional application No. 63/129,966, filed on Dec. 23, 2020.

(51) Int. Cl. A61L 27/36; A61L 27/54

(52) U.S. Cl. CPC A61L 27/3633; A61L 27/3641; A61L 27/3687; A61L 27/54; A61L 2400/06; A61L 2430/20

(57) ABSTRACT

This disclosure describes a method for altering the proteome of a diseased heart, organ or tissue by contacting it with extracellular matrix (ECM) particles derived from decellularized 3D microtissues. In an example, the process involves restoring the normal ratio of collagen levels, modulating immune responses, improving mitochondrial function, and/or mitigating the effects of TGF-Beta, a growth factor associated with fibrosis. The ECM particles can be produced from human mesenchymal stem cells and retain bioactive components and structural properties of native tissue. Additionally, the disclosure outlines a composition for treating a diseased organ using these ECM particles, which are treated to enhance crosslinking, stability, injectability, and compatibility with patients.

### CROSS-REFERENCE TO RELATED APPLICATIONS

This patent application is a continuation in part (CIP) of U.S. patent application Ser. No. 18/339,980, filed 22 Jun. 2023, which is a continuation of International PCT Application No. PCT/US2021/064834, filed on 22 Dec. 2021, and published as WO 2022/140530 A1 on 30 Jun. 2022, which claims priority to U.S. provisional patent application 63/129,966, filed 23 Dec. 2020, and the contents of all of which are incorporated herein by reference as if each was set forth herein in its entirety. This CIP patent application claims the benefit of priority to U.S. Provisional Patent No. 63/129,966, filed 23 Dec. 2020, the entire disclosure of which is incorporated by reference as if fully set forth herein in its entirety. This patent application also claims the benefit of priority to U.S. Provisional Patent No. 63/560,539, filed 1 Mar. 2024, the entire disclosure of which is incorporated by reference as if fully set forth herein in its entirety.

### STATEMENT REGARDING FEDERALLY SPONSORED RESEARCH OR DEVELOPMENT

This invention was made with government support under grant number R03 EB028056 awarded by the National Institutes of Health. The government has certain rights in the invention.

### FIELD OF THE INVENTION

The present disclosure generally relates to the field of tissue regeneration and repair, particularly utilizing decellularized mammalian extracellular matrix (ECM) particles and changes to healing and/or to a proteome that can arise during treatments.

### BACKGROUND OF THE INVENTION

Organ failure, such as resulting from disease or trauma, poses substantial health and cost issues to society. For example, successful treatment often requires the repair or replacement of the organ, but an increasing shortage of transplantable organs has resulted in a wait-list of over 100,000 patients in the US alone. The situation is particularly dire for patients with cardiovascular disease; approximately 790,000 Americans suffer a myocardial infarction (MI) each year. While up to 50% of MI patients survive, all will have sustained progressive cardiac tissue damage, and this progressive damage is a leading cause of mortality for MI survivors worldwide. Indeed, many survivors subsequently develop heart failure, for which the 5-year survival rate is only 50%.

No current treatments can prevent post-MI heart failure; heart transplants and left ventricular assist devices are the only options for end-stage heart failure. Both options are expensive and have limitations, including scarcity of donor organs.

The heart possesses regeneration potential derived from endogenous and exogenous stem and progenitor cell populations, though baseline regeneration appears to be sub-therapeutic. This limitation was initially attributed to a lack of cells with cardiomyogenic potential following an insult to the myocardium. However, recent studies demonstrate increased numbers of cardiomyocyte progenitor cells in diseased hearts. Given that the limiting factor does not appear to be cell quantity but rather repletion of functional cardiomyocytes, it is crucial to understand potential mechanisms inhibiting progenitor cell differentiation. An area of interest in heart disease treatment is extracellular matrix (ECM) remodeling, with both the composition and mechanical properties of the ECM undergoing changes in diseased hearts.

Alternative treatments utilizing tissue engineering could help bridge the gap between available organs and patients in need of new organs to survive. For example, a natural scaffold, ECM, can support tissue repair and regeneration. Decellularized organ tissue from animals can be processed into an injectable liquid that solidifies into a gel at the site of injection. However, the ECM from animals is composed of biomolecules and protein sequences foreign to humans that can elicit an immune reaction that limits effectiveness or even causes further tissue damage. At the same time, it has been challenging to extract ECM from human-derived tissues, and various pathologies and post-mortem degradation of human tissue also render them unacceptable ECM material for tissue repair.

Accordingly, there is a need for novel sources and methods of preparing decellularized mammalian, for example human, ECM morsels (spheroid-shaped microtissues) for use in tissue repair and regeneration.

### BRIEF SUMMARY OF THE INVENTION

The present disclosure, in part, relates to novel sources, compositions, and methods of preparing and using decellularized mammalian ECM particles or morsels. Generally, the methods and compositions disclosed herein relate to the use of human ECM for the purpose of tissue repair and regeneration, for example in therapeutic or cosmetic procedures.

Disclosed embodiments comprise three-dimensional (3D) in-vitro cell culture systems to engineer specifically-tailored microtissues suitable for particular patients and indications. In embodiments, the three-dimensional (3D) in-vitro cell culture systems comprise scaffold-free systems.

Disclosed tissues can be decellularized to fabricate decellularized mammalian extracellular matrix (ECM), for example in the form of small porous particles or morsels that are equal to or less than 800 um in diameter, thus providing “flowable” compositions that can be administered with, for example, a cannula such as a needle. Disclosed compositions can be administered via injection due to the size and shape of the ECM particles/morsels.

The resulting decellularized mammalian ECM particles/morsels can have a number of clinical applications including but not limited to supporting tissue regeneration.

Disclosed herein are customized decellularized mammalian ECM compositions. For example, in embodiments, disclosed compositions are derived from cultured human cells. In embodiments the human cells can comprise heart or lung cells. In embodiments, the human cells can comprise recombinant human cells.

Disclosed herein are methods of producing customized decellularized ECM compositions, for example mammalian ECM compositions. For example, in embodiments, disclosed methods comprise production of ECM derived from cultured human cells. In embodiments the human cells can comprise heart or lung cells. In embodiments, the human cells can comprise recombinant human cells. Disclosed methods provide for faster, more efficient decellularization as compared to methods previously known in the art.

Disclosed herein are methods of using customized decellularized mammalian ECM compositions. For example, in embodiments, disclosed methods of use comprise, for example, repair and regeneration of, for example, a cardiovascular injury in, for example, a mammal such as a human. In embodiments, disclosed methods of use can comprise administration of a disclosed ECM composition to a treatment area, for example the heart. In embodiments, disclosed compositions can be administered via injection, as a liquid such as an aerosol, or as an impregnated patch. In embodiments, disclosed flowable compositions can be administered using a cannula such as a needle, for example a syringe.

As an additional brief summary, for example, to provide discussion points for a brief summary, some features of the technology disclosed herein can be briefly summarized (or discussed) by the following list of features, any of which can be inter-combined with any other feature, detail, embodiment, discussion point, aspect, or example disclosed herein:

Feature 1: A method of altering the proteome of a diseased heart, comprising: contacting the diseased heart with extra-cellular matrix (ECM) particles produced from decellularized 3D microtissues, wherein the contacting alters the proteome of the diseased heart.

Feature 2: The method of feature 1, wherein the contacting restores a normal ratio of levels of different collagens in the heart. Feature 3: The method of feature 1, wherein the contacting alters an immune response in the heart. Feature 4: The method of feature 1, wherein the contacting alters metabolism in the heart. Feature 5: The method of feature 1, wherein the contacting mitigates effects of TGF-Beta in the heart, wherein TGF-Beta is a growth factor associated with fibrosis. Feature 6: The method of feature 1, wherein the 3D microtissues are derived from human mesenchymal stem cells.

Feature 7: A method of treating a diseased heart, comprising: administering to the diseased heart extra-cellular matrix (ECM) particles produced from decellularized 3D microtissues, wherein the administering treats the diseased heart by altering the proteome of the diseased heart.

Feature 8: The method of feature 7, wherein the administering restores a normal ratio of levels of different collagens in the heart. Feature 9: The method of feature 7, wherein the administering alters an immune response in the heart. Feature 10: The method of feature 7, wherein the administering alters metabolism in the heart. Feature 11: The method of feature 7, wherein the administering mitigates effects of TGF-Beta in the heart, wherein TGF-Beta is a growth factor associated with fibrosis. Feature 12: The method of feature 7, wherein the 3D microtissues are derived from human mesenchymal stem cells.

Feature 13: Extra-cellular matrix (ECM) particles for altering the proteome of a diseased heart (i.e., or organ and/or tissue), wherein the ECM particles are produced from decellularized 3D microtissues and treated to improve one or more of crosslinking, stability, injectability, or compatibility with a patient.

Feature 14: The ECM particles of feature 13, wherein the ECM particles, when contacted with the diseased heart, restore a normal ratio of levels of different collagens in the heart. Feature 15: The ECM particles of feature 13, wherein the ECM particles, when contacted with the diseased heart, alter an immune response in the heart. Feature 16: The ECM particles of feature 13, wherein the ECM particles, when contacted with the diseased heart, alter metabolism in the heart. Feature 17: The ECM particles of feature 13, wherein the ECM particles, when contacted with the diseased heart, mitigate effects of TGF-Beta in the heart, wherein TGF-Beta is a growth factor associated with fibrosis. Feature 18: The ECM particles of feature 13, wherein the 3D microtissues are derived from human mesenchymal stem cells.

Feature 19: A composition for treating a diseased heart, comprising: extra-cellular matrix (ECM) particles produced from decellularized 3D microtissues and treated to improve one or more of crosslinking, stability, injectability, or compatibility with a patient; wherein the composition, when administered to the diseased heart, treats the diseased heart by altering the proteome of the diseased heart.

Feature 20: The composition of feature 19, wherein the 3D microtissues are derived from human mesenchymal stem cells. In any of the features above, the term “heart” refers to any organ and/or tissue in a human body.

While the summary examples disclosed above provide some introduction to embodiments of the invention, other implementations are also contemplated, described, and recited herein. These and other features and advantages will be apparent from a reading of the following detailed description, the Examples, and a review of the associated drawings. It is to be understood that both the foregoing general description and the following detailed description are explanatory only and are not restrictive of aspects as claimed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B illustrate the ECM (FIG. 1A), a fibrous network of proteins, proteoglycans, and glycoproteins arranged in a three-dimensional (3D) architecture, and its uses in human tissue regeneration after damage, such as a myocardial infarction (FIG. 1B).

FIGS. 2A-2D illustrate a method of generating spheroid-shaped microtissues for fabricating human ECM morsels. The process begins by seeding cultured human cells into micro-wells using a non-adhesive micro-mold platform technology (FIGS. 2A and 2B). See, e.g., U.S. Pat. No. 8,361,781 (Morgan et al.), herein incorporated by reference in its entirety. The seeded cells aggregate, synthesize, assemble, and deposit human ECM. Resulting ECM microtissues or spheroids (each approximately 50 to 300 μm in diameter) within each of the micro-wells (each approximately 400-800 μm in diameter) are illustrated in FIGS. 2C and 2D.

FIGS. 3A-3B illustrate disclosed non-adhesive micro-mold platform technology, including schematics of a master mold (FIG. 3A), as well as the corresponding 3D printed mold, the silicone negative, and an image of an agarose mold with ECM microtissue within the micro-wells. FIG. 3B illustrates another embodiment comprising ring- and honeycomb-shaped molds, the corresponding negative replicates in agarose, and the resulting ring- and honeycomb-shaped 3D human ECM microtissues (6 million cells, 2 cm across), removed from the agarose gel and stained for viable cells with calcein-AM. Also illustrated is a decellularized honeycomb-shaped 3D tissue of human dermal fibroblasts (14 million cells) showing ECM made after only 3 days; 2 cm across.

FIG. 4 illustrates a method of generating decellularized human ECM morsels. In FIG. 4 fetal and adult human heart cells or adult lung cells can be cultured and seeded into micro-wells to generate ECM microtissue or spheroids. The ECM spheroid microtissues can be decellularized inside the micro-wells. The resulting decellularized fetal and adult human heart or human lung ECM morsels maintain their spheroid shape following the decellularization procedure. Scale bar=200 μm.

FIG. 5 illustrates an additional or alternative method of generating decellularized human ECM morsels. In FIG. 5 fetal and adult human heart cells can be cultured and seeded into micro-wells to generate ECM microtissue or spheroids. The ECM spheroid microtissues can be collected, decellularized, and mixed, for example through vortexing. The resulting decellularized fetal and adult human heart ECM morsels maintain their spheroid shape following the decellularization procedure.

FIGS. 6A-6B illustrate decellularized human fetal ECM morsels before and after passage through a 27G syringe 10 times (FIG. 6A), or imaged with an inverted microscope in brightfield with a 10× objective before and after passage through a 27G syringe one time (FIG. 6B).

FIG. 7A illustrates one embodiment, showing stained ECM spheroid microtissues from human lung cells, either from healthy cells or cells from a patient with idiopathic pulmonary fibrosis (IPF), either untreated or treated with transforming growth factor beta 1, and FIG. 7B illustrates ECM spheroid microtissues from adult and fetal human heart cells (fibroblasts alone, or co-cultured with cardiac myocytes and microvascular endothelial cells). Fibroblast-only adult or fetal heart ECM microtissues were cultured for 3, 6, 9 or 12 days, whereas the tri-cultures were only cultured for 6 days. Images of the ECM spheroid microtissues in brightfield, stained with hematoxylin and eosin (H&E) or SIRIUS RED™, before decellularization are illustrated and ECM spheroid microtissues from adult and fetal human heart or adult lung cells after decellularization are also illustrated. Scale bars=50 μm.

FIG. 8 illustrates that the size increase of human fetal heart microtissues from day 3 to 12 is associated with increases in collagen (μg) and sulphated glycosaminoglycans (sGAG) (μg) content, but not with changes in DNA content (ng/mL).

FIGS. 9A-9C shows lung and heart ECM microtissues under multi-photon confocal second-harmonic generation (SHG) microscopy to capture fibrillar collagen architecture in three dimension. Human adult healthy lung ECM microtissues (FIG. 9A) reveal different fibrillar collagen structure than human adult fibrotic lung ECM (FIG. 9B) and human adult healthy heart ECM microtissues (FIG. 9C).

FIG. 10 Mechanical stiffness of cultured human fetal heart ECM is comparable to healthy human heart tissue. Graphical representation of cultured human ECM elastic moduli (mean±SD kPa) of adult lung (L-A), adult heart (H-A) and fetal heart (H-F), as compared to the stiffness range for healthy human heart (shaded in red), and the equivalent elastic modulus of gelled collagen (Col), Matrigel (Mat), not-crosslinked porcine cardiac ECM (Pig-N) and crosslinked porcine cardiac ECM (Pig-C) as reported in published work from another laboratory. N=9/group.

FIG. 11 shows that cultured ECM is biocompatible in vitro. Representative images of fixed and embedded sections of human cardiac myocyte (HCM) or microvascular endothelial cells (HCMEC) seeded into microwell with or without cultured human adult or fetal heart ECM that were incubated with EdU (5-ethynyl-2′-deoxyuridine) 24 h post cell-seeding for 48 h (HCM) or 24 h (HCMEC) prior fixation. Sections were stained with H&E or Click-iT™ EdU Cell Proliferation Kit, where proliferating cells were labelled with Alexa Fluor™ 488 dye (red) and all nuclei were counterstained in Hoechst 33342 (blue). N=3.

FIGS. 12A-12B shows molds for making micro-molded agarose gels. FIG. 12A shows a commercially available mold on the left that makes an agarose gel with 256 micro-wells. Each gel fits in one well of a 12-well plate. A custom mold in on the right makes an agarose gel with 721 micro-wells. Each gel fits in one well of a 6-well plate. FIG. 12B shows a custom “master mold” designed to produce multiple copies of the mold for 6 well plates.

FIGS. 13A-13C show the principal component analysis (PCA) of MSC ECM. FIG. 13A shows protein intensities by batch and replicate. FIG. 13B shows protein intensity by ECM component for three batches of MSC ECM. FIG. 13C shows the number of proteins by ECM component.

FIGS. 14A-14F show intensity of ECM components: collagens, glycoproteins, proteoglycans, ECM regulators, secreted factors and ECM affiliated proteins.

FIG. 15 shows the top 50 core matrisome (green circles), matrisome-associated (blue circles), and non-matrisome proteins (red circles) in MSC ECM particles with stringDB interaction map highest confidence interactions (StringDB interaction score ≥0.9). Disconnected nodes are removed. Abbreviations: ACTA2—actin alpha 2, smooth muscle; ACTB—actin beta; AGRN—agrin; ALB—albumin; ANXA1—annexin A1; ANXA11—annexin A11; ANXA2—annexin A2; ANXA5—annexin A5; ANXA6—annexin A6; BGN—biglycan; BMP1—bone morphogenetic protein 1; CFL1—cofilin 1; COL11A2—collagen type XI alpha 2 chain; COL14A1—collagen type XIV alpha 1 chain; COL18A1—collagen type XVIII alpha 1 chain; COL1A1—collagen type I alpha 1 chain; COL1A2—collagen type I alpha 2 chain; COL2A1—collagen type II alpha 1 chain; COL3A1—collagen type III alpha 1 chain; COL4A1—collagen type IV alpha 1 chain; COL4A2—collagen type IV alpha 2 chain; COL5A1—collagen type V alpha 1 chain; COL5A2—collagen type V alpha 2 chain; COL5A3—collagen type V alpha 3 chain; COL6A1—collagen type VI alpha 1 chain; COL6A2—collagen type VI alpha 2 chain; COL6A3—collagen type VI alpha 3 chain; COL9A1—collagen type IX alpha 1 chain; COL9A2—collagen type IX alpha 2 chain; CSTA—cystatin A; CSTB—cystatin B; CTSB—cathepsin B; CTSD—cathepsin D; DCN—decorin; EEF1A1—eukaryotic translation elongation factor 1 alpha 1; EEF2—eukaryotic translation elongation factor 2; EIF4A1—eukaryotic translation initiation factor 4A1; FBLN1—fibulin 1; FBN1—fibrillin 1; FLG2—filaggrin 2; FN1—fibronectin 1; GAPDH—glyceraldehyde-3-phosphate dehydrogenase; FLG—filaggrin; H1-4—H1.4 linker histone, cluster member; H1-5—H1.5 linker histone, cluster member; H2AC11—H2A clustered histone 11; H2BC14—H2B clustered histone 14 [Source: HGNC Symbol; Acc: HGNC: 4750]; H2BC20P—H2B clustered histone 20, pseudogene; H3C1—H3 clustered histone 1; H4C1—H4 clustered histone 1 [Source: HGNC Symbol; Acc: HGNC: 4781]; HNRNPC—heterogeneous nuclear ribonucleoprotein C; HNRNPK—heterogeneous nuclear ribonucleoprotein K; HNRNPU—heterogeneous nuclear ribonucleoprotein U; HSP90AB1—heat shock protein 90 alpha family class B member 1; HSP90B1—heat shock protein 90 beta family member 1; HSPA5—heat shock protein family A (Hsp70) member 5; HSPA8—heat shock protein family A (Hsp70) member 8; HSPB1—heat shock protein family B (small) member 1; HSPG2—heparan sulfate proteoglycan 2; KRT10—keratin 10; KRT14—keratin 14; KRT2—keratin 2; KRT5—keratin 5; LAMB1—laminin subunit beta 1; LAMC1—laminin subunit gamma 1; LGALS3—galectin 3; LTBP1—latent transforming growth factor beta binding protein 1; LUM—lumican; MMP14-matrix metallopeptidase 14; MMP2—matrix metallopeptidase 2; MYH9—myosin heavy chain 9; NID2—nidogen 2; P4HA1—prolyl 4-hydroxylase subunit alpha 1; P4HA2—prolyl 4-hydroxylase subunit alpha 2; PFN1—profilin 1; PKM—pyruvate kinase M1/2; PLOD1—procollagen-lysine,2-oxoglutarate 5-dioxygenase 1; PLOD2—procollagen-lysine,2-oxoglutarate 5-dioxygenase 2; PLOD3—procollagen-lysine,2-oxoglutarate 5-dioxygenase 3; POSTN—periostin; PXDN—peroxidasin; RAN—RAN, member RAS oncogene family; RPL11—ribosomal protein L11; RPS16—ribosomal protein S16; RPS25—ribosomal protein S25; RPS27A—ribosomal protein S27a; RPS3—ribosomal protein S3; RPS8—ribosomal protein S8; RPSA—ribosomal protein SA; S100A10—S100 calcium binding protein A10; S100A11—S100 calcium binding protein A11; S100A14—S100 calcium binding protein A14; S100A16—S100 calcium binding protein A16; S100A4—S100 calcium binding protein A4; S100A6—S100 calcium binding protein A6; S100A8—S100 calcium binding protein A8; S100A9—S100 calcium binding protein A9; SERPINH1—serpin family H member 1; SPARC—secreted protein acidic and cysteine rich; TGFBI—transforming growth factor beta induced; TGM1—transglutaminase 1; TGM2—transglutaminase 2; THBS1—thrombospondin 1; TUBA1A—tubulin alpha 1a; TUBB—tubulin beta class I; TUBB3—tubulin beta 3 class III; TUBB4B—tubulin beta 4B class lvb; and VCAN—versican.

FIG. 16 provides a comparison of decellularized mammalian ECM particles or morsels from cultured human cells such as heart or lung cells with MSC ECM particles.

FIGS. 17A-17B show TGFβ pathway regulators. FIG. 17A shows the interaction network of TGFβ signaling pathway regulators in EMC particles. FIG. 17B show intensities of TGFβ pathway regulators in ECM particle batch proteomics. Positive regulators are shown in blue and negative regulators are shown in red. The width of the lines joining the TGFβ pathway regulators indicate the interaction confidence, with broad, dark lines indicating high interaction confidence and lighter, thin lines indicating low interaction confidence. Abbreviations: CD109—CD109 molecule; DNM2—dynamin 2; EMILIN1—elastin microfibril interfacer 1; FBN1—fibrillin 1; GIPC1—GIPC PDZ domain containing family member 1; GLG1—golgi glycoprotein 1; GOT1—glutamic-oxaloacetic transaminase 1; HDAC1—histone deacetylase 1; HDAC3—histone deacetylase 3; HSPA5—heat shock protein family A (Hsp70) member 5; HTRA1—HtrA serine peptidase 1; LEMD3—LEM domain containing 3; LRP1—LDL receptor related protein 1; LTBP1—latent transforming growth factor beta binding protein 1; NRP1—neuropilin 1; OGT—O-linked N-acetylglucosamine (GlcNAc) transferase; PEG10—paternally expressed 10; PIN1—peptidylprolyl cis/trans isomerase, NIMA—interacting 1; RBBP4—RB binding protein 4, chromatin remodeling factor; RBBP7—RB binding protein 7, chromatin remodeling factor; SDCBP—syndecan binding protein; SIN3A—SIN3 transcription regulator family member A; SNW1—SNW domain containing 1; SNX1—sorting nexin 1; SNX6—sorting nexin 6; STRAP—serine/threonine kinase receptor associated protein; STUB1—STIP1 homology and U-box containing protein 1; SUDS3—SDS3 homolog, SIN3A corepressor complex component; TGFB1I1—transforming growth factor beta-1-induced transcript 1 protein; THBS1—thrombospondin 1; and USP15—ubiquitin specific peptidase 15.

FIGS. 18A-18D show molecular factors of adverse remodeling after myocardial infarction (MI) and factors for healthy recovery provided by MSC ECM particles injected after MI.

FIG. 19 shows a histogram profile of MSC ECM particle proteins. Abbreviations: ITGAV—integrin subunit alpha V; COL5A3—collagen type V alpha 3 chain; LTBP1—latent transforming growth factor beta binding protein 1; COL5A1—collagen type V alpha 1 chain; COL5A2—collagen type V alpha 2 chain; EMILIN1—elastin microfibril interfacer 1; DCN—decorin; COL6A3—collagen type VI alpha 3 chain; COL3A1—collagen type III alpha 1 chain; COL1A2—collagen type I alpha 2 chain; COL1A1—collagen type I alpha 1 chain.

FIGS. 20A-20C show effects of MSC ECM particles on cardiac tissue. Control injection samples are represented in grey and MSC ECM particles treated hearts are represented in magenta. FIG. 20A echocardiographic data pre-op and POD 3, 7, 14, 21, and 28. FIG. 20B shows the analysis of percent LV infarct and representative images of Masson's trichrome stain.

FIG. 20C shows dual immunofluorescence microscopy of isolectin B4 (endothelial cells) and αSMA (arterioles). Left panels show quantitative analysis of capillaries (isolectin B4) and arterioles (alpha-SMA) in control and saECM hearts (n=6).

FIG. 21A shows a plot of Ischemic and perfused regions. FIG. 21B shows a plot of Ischemic normalized to perfused regions.

FIG. 22A shows a plot of Cellular fraction; Ischemic region (ECM particles vs. saline injected). FIG. 22B shows a plot of Cellular fraction; Perfused region. FIG. 22C shows a plot of Soluble fraction/Ischemic region. FIG. 22D shows a plot of Soluble fraction/Perfused region.

FIG. 22E shows a plot of Insoluble fraction/Ischemic region. FIG. 22F shows a plot of Insoluble fraction/Perfused region.

FIG. 23A shows Ischemic and perfused regions, Average protein density. FIG. 23B shows Ischemic and perfused regions, ECM-vs. Saline-injected average protein intensity.

FIG. 23C shows an example heat map for region, fraction vs. protein for Perfused, Cellular. FIG. 23D shows region, fraction vs. protein for Ischemic, Cellular. FIG. 23E shows region, fraction vs. protein for Perfused, sECM. FIG. 23F shows a heat map for region, fraction fs. protein for Ischemic, sECM. FIG. 23G shows heatmaps for Perfused, iECM (top) and Ischemic, iECM (bottom).

For the mouse heart proteomics study ischemic and perfused regions, FIG. 24A shows the matrisome category composition by fractionation proteomics, cellular fraction, for the Ischemic region. FIG. 24B shows the matrisome category composition by fractionation proteomics, cellular fraction, for the Perfused region.

FIG. 24C shows the matrisome category composition by fractionation proteomics, soluble fraction, for the Ischemic region. FIG. 24D shows the matrisome category composition by fractionation proteomics, soluble fraction, for the Perfused region.

FIG. 24E shows the matrisome category composition by fractionation proteomics, insoluble fraction, for the Ischemic region. FIG. 24F shows the matrisome category composition by fractionation proteomics, insoluble fraction, for the Perfused region.

FIG. 25A shows the biological process enrichment of significantly upregulated or downregulated proteins from ischemic regions of hearts injected with ECM particles vs. saline.

FIG. 25B shows the biological process enrichment of significantly upregulated or downregulated proteins from ischemic regions of hearts injected/tested. FIG. 25C shows the biological process enrichment of significantly upregulated or downregulated proteins from perfused regions of hearts injected/tested. FIG. 25D shows the biological process enrichment of significantly upregulated or downregulated proteins from perfused regions of hearts injected/tested.

FIG. 26A shows the Cellular fraction plot for ECM particles vs. saline injected. FIG. 26B shows the Soluble fraction plot for ECM particles vs. saline injected. FIG. 26C shows the Insoluble fraction plot for ECM particles vs. saline injected.

FIG. 27A shows a heat map of Ischemic normalized to perfused region, Average protein intensity. FIG. 27B shows a heat map of Ischemic normalized to perfused region. FIG. 27C shows a heat map of Combined fractions. FIG. 27D shows a heat map of Cellular. FIG. 27E shows a heat map of sECM. FIG. 27F shows a heat map of iECM.

FIG. 28A shows matrisome category composition by fractionation proteomics: ischemic normalized to perfused region, for Cellular fraction. FIG. 28B shows matrisome category composition by fractionation proteomics: ischemic normalized to perfused region, for Soluble fraction. FIG. 28C shows matrisome category composition by fractionation proteomics: ischemic normalized to perfused region, for Insoluble fraction.

FIG. 29A shows upregulated proteins for the gene ontology biological process analyses (significantly differential proteins). FIG. 29B shows downregulated proteins for the gene ontology biological process analyses (significantly differential proteins).

FIG. 30A, FIG. 30B, FIG. 30C, and FIG. 30D show example components (Table 3 in parts) in a composition of SFM+ medium based on DMEM, SFMA based on Advanced DMEM and 50:50 medium.

FIG. 31 provides an example flowchart illustrating a method in step for altering the proteome of a diseased heart by contacting it with extra-cellular matrix (ECM) particles produced from decellularized 3D microtissues, according to an embodiment. FIG. 32 is a flowchart illustrating a method in step for administering extra-cellular matrix (ECM) particles to a diseased heart, according to an embodiment.

It should be understood that while different illustrations are sometimes used in some of the figures above to describe different embodiments and different aspects of the technology, any aspect from any figure can be inter-combined with an aspect from any other figures. Any example disclosed herein can be inter-combined with any other. All trademarks, images, likenesses, words, and depictions in the drawings and the disclosure are plainly in fair use and are provided solely for the purposes of illustration of the invention in view of an urgent need to prevent injuries and to treat subjects as further discussed in more detail below.

### DETAILED DESCRIPTION OF THE INVENTION

Some definitions are provided hereafter. Nevertheless, definitions may be located in the “Embodiments” section below, and the above header “Definitions” does not mean that such disclosures in the “Embodiments” section are not definitions.

### Definitions

“Administration,” or “to administer” means the step of giving (i.e. administering) a medical device, material or agent to a subject. The materials disclosed herein can be administered via a number of appropriate routes, but are typically employed in connection with a surgical procedure.

As used herein in the specification and the appended claims, the singular forms “a,” “an,” and “the” include plural referents unless the context clearly dictates otherwise. Thus, for example, reference to “a component” or “the component” includes two or more components.

The term “and/or” used in the context of “X and/or Y” should be interpreted as “X,” or “Y,” or “X and Y.” Similarly, “at least one of X or Y” should be interpreted as “X,” or “Y,” or “X and Y.”

“ECM physical property” refers to properties including but not limited to the shape, size, surface roughness, porosity, fibrillar collagen two-dimensional architecture, fibrillar collagen three-dimensional architecture, of the ECM morsels/particles.

“ECM biochemical property” refers to properties including but not limited to species (identity) and contents (relative amounts) of biochemical molecules (amino acids, peptides, proteins, modified proteins, carbohydrates, fatty acids, glycosaminoglycans, enzymes, signalling molecules (such as transforming growth factor beta 1), cytokines, hormones), as well as the degradability and biocompatibility of ECM morsels/particles.

“ECM mechanical property” refers to properties including but not limited to tensile strength, compressive strength, elastic modulus, shear modulus of ECM morsels/particles.

“In-vivo ECM properties” refers to physical, biochemical, or mechanical properties associated with naturally-occurring ECM.

“Customized ECM” refers to ECM with physical, biochemical, or mechanical properties that differ from those associated with naturally-occurring ECM as, such difference a result of the disclosed methods.

“ECM microtissue” refers to 3D compositions comprising cells and ECM.

“ECM morsels” or “ECM particles” refers to decellularized ECM microtissue.

Ranges can be expressed herein as from “about” or “approximately” one particular value, and/or “about” or “approximately” to another particular value. When such a range is expressed, another embodiment includes from the one particular value and/or to the other particular value. Similarly, when values are expressed as approximations, by use of the antecedent “about” or “approximately,” it will be understood that the particular value forms another embodiment.

The terms “subject” and “patient” are used interchangeably and refer to any individual who is the target of administration or treatment. The “subject” can be a vertebrate, such as a mammal. The “subject” can be a human or veterinary patient. The term “patient” generally refers to a “subject” under the treatment of a clinician, e.g., physician, or a healthcare professional.

The terms “peptide,” “polypeptide,” and “protein” are used interchangeably to refer to a polymer of amino acid residues.

The term “therapeutically effective” refers to the amount of the composition used that is of sufficient quantity to ameliorate one or more causes or symptoms of a disease or disorder. Such amelioration only requires a reduction or alteration, not necessarily elimination. In addition, the term “therapeutically effective” includes the amount of the composition used is of sufficient quantity to initiate and/or support the body's tissue or organ repair processes.

The term “treatment” refers to the medical management of a patient with the intent to cure, ameliorate, stabilize, prevent a disease, pathological condition, or disorder. In addition, the term “treatment” refers to the medical management of a patient with the intent to repair, regenerate, or provide support for the body's repair or regenerative processes, for an injury, tissue damage, or organ damage. This term includes active treatment, that is, treatment directed specifically toward the improvement of a disease, injury, damage, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, injury, or disorder; and supportive treatment, that is, treatment employed to supplement another specific therapy directed toward the improvement of the associated disease, pathological condition, injury, or disorder.

The term “administration” to a subject includes any route of introducing or delivering to a subject an agent. “Administration” can be carried out by any suitable route, including, but not limited to oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like.

This disclosure can save human lives, thus the terms “heart” and/or “diseased heart” can refer to any organ, tissue, and/or cell in a human body that can be treated to improve a condition, sign and/or symptom of a subject in need thereof. The term “crosslinking” used herein can refer to an ECM (extra cellular matrix) made by growing a human cell using a cell culture medium not found in nature into a cell spheroid, then decellularizing the spheroid while retaining the ECM which was grown in vitro using the cell culture medium, which ECM will not be found in a human because the ECM grown in the cell culture medium has a different (e.g., controlled, tailored) crosslinking than any ECM found in nature. The terms “treat,” “treating,” “treatment,” and grammatical variations thereof as used herein, include the administration of a composition with the intent or purpose of partially or completely preventing, delaying, curing, healing, repairing, regenerating, alleviating, relieving, altering, remedying, ameliorating, improving, stabilizing, mitigating, and/or reducing the intensity or frequency of one or more diseases or conditions, a symptom of a disease or condition, or an underlying cause of a disease or condition. Treatments according to the invention may be applied preventively, prophylactically, palliatively or remedially.

### EMBODIMENTS

Various non-exhaustive, non-limiting aspects of compositions according to the present disclosure may be useful alone or in combination with one or more other aspects described herein. Disclosed systems, compositions and methods provide unique advantages to both patients and practitioners. For example, disclosed embodiments can produce customized 3D microtissues specifically tailored for use with a particular patient and/or for a particular treatment. In embodiments, these microtissues can be used to fabricate decellularized mammalian ECM particles/morsels from many different types and combinations of mammalian cells, including and not limited to lung fibroblasts, dermal fibroblasts, cardiac fibroblasts, cardiac microvascular endothelial cells, cardiac myocytes of different ages (adult, fetal, juvenile, etc.). Thus, provided herein are methods for producing customizable ECM in-vitro, including ECM that cannot be made from in-vivo tissues.

The biochemical/chemical composition (both biochemical/chemical species and/or their contents), collagen architecture, and mechanical properties of the ECM particles/morsels generated by the mammalian cells in microtissues are uniquely dependent on the types of mammalian cells used (tissue origin, age, disease state, etc.). Thus, particular cells can be employed to achieve desired ECM properties.

The biochemical/chemical composition (both biochemical/chemical species and/or their contents), collagen architecture, and mechanical properties of the ECM particles/morsels generated by the mammalian cells in microtissues are further dependent on the culturing conditions of the mammalian cells as they form microtissues. For example, cell culture media composition, culturing time, oxygen level, and the presence or amount of additional biological factors including and not limited to growth factors, cytokines, drugs, and the like can be adjusted to produce the desired ECM properties.

The biochemical/chemical composition (both biochemical/chemical species and/or their contents), collagen architecture, and mechanical properties of the disclosed ECM particles/morsels generated by the mammalian cells in microtissues can be different from ECM extracted from mammalian tissues found in nature (for example, pig heart, human heart, etc.).

Disclosed ECM particles/morsels can form flowable compositions that can pass through a syringe with an attached needle, where the needle inner diameter (ID) would depend on the size of the ECM particles/morsels. For example, ECM particles/morsels with a diameter of ˜200 um will be able to pass through any needle with an ID equal or larger than the ID of a 27G needle (ID=210 um).

Disclosed ECM particles can be made using fewer steps than ECM extracted from animal or human tissues and organs. Disclosed ECM particles can be made using aseptic conditions.

Disclosed decellularized mammalian ECM particles/morsels are biocompatible in-vitro, such that mammalian cells placed on the decellularized mammalian ECM particles/morsels can survive and multiply.

Different ECM characteristics (including, for example, physical properties, biochemical/chemical compositions, collagen architecture, mechanical properties, and combinations thereof) are likely to have different effects on surrounding tissue once implanted in-vivo, allowing for customized ECM to be developed for any number of clinical conditions.

### ECM Compositions

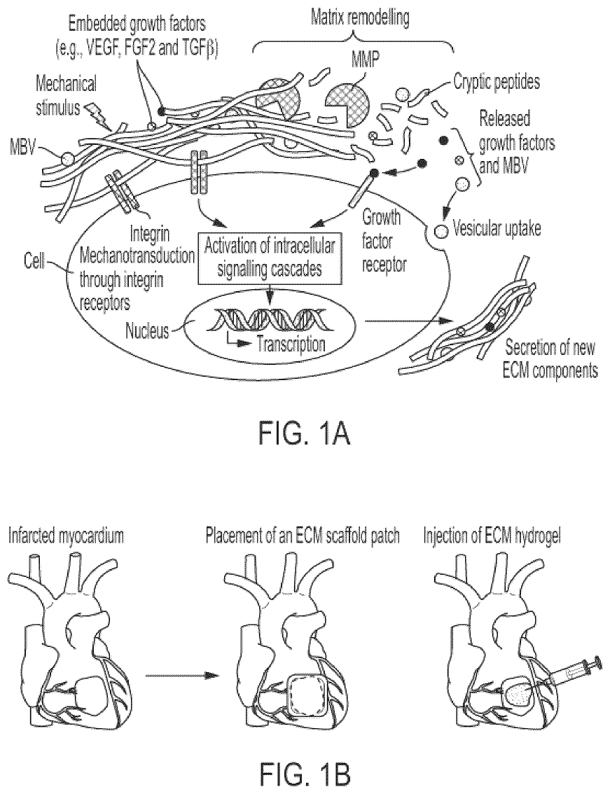

Disclosed herein are customized decellularized compositions, for example decellularized mammalian ECM compositions, whose properties can be specifically tailored to suit particular patients (or patient groups) as well as particular indications. FIG. 1 illustrates the ECM (FIG. 1A), a fibrous network of proteins, proteoglycans, and glycoproteins arranged in a three-dimensional (3D) architecture, and its uses in human tissue regeneration after damage, such as a myocardial infarction (FIG. 1B). The use of foreign, non-human decellularized ECM for tissue regeneration and/or repair can be prone to causing immune reactions in the subject. Decellularized human ECM morsels overcome the issue of immune reactions because the ECM is human. Thus, in embodiments, the decellularized cell are human cells. In embodiments, the human cells can comprise heart or lung cells. In embodiments, the human cells can comprise recombinant human cells. In embodiments, the human cells can comprise at least one of cardiac fibroblasts, cardiac myocytes and cardiac microvascular endothelial cells.

In embodiments, disclosed compositions are derived from cultured human cells used to form decellularized human ECM morsels. The ECM can comprise a complex 3D architecture of structural proteins such as collagen and elastin, along with proteoglycans, enzymes and growth factors (FIG. 1A). In embodiments, the ECM provides structural support, as well as signals for tissue regeneration (FIG. 1B).

ECM Compositions-Methods of Production: Disclosed herein are methods of producing customized decellularized mammalian-derived ECM compositions with desired physical or chemical properties. For example, in embodiments, disclosed methods are derived from cultured human cells.

Disclosed methods can comprise: Seeding cultured cells (such as mammalian cells) into micro-wells, wherein the cultured mammalian cells generate 3D microtissues, where each of the microtissues are comprised of the cultured mammalian cells and the cell-secreted soluble and insoluble ECM; Collecting microtissues; and Decellularizing microtissues to form ECM morsels or particles with steps that involve in combining the microtissues with detergent, a buffer, a DNase, and an RNase, and mixing/vortexing the mixture.

Various human cell lines can be utilized as sources for disclosed decellularized human ECM morsels. In embodiments the human cells can comprise, for example, heart or lung cells. In embodiments, the human cells can comprise recombinant human cells. For example, in embodiments, human cell lines utilized as sources can include cardiac fibroblasts, cardiac myocytes, cardiac microvascular endothelial cells, and lung fibroblasts. In addition, other human cell types and of different maturation could be used as a source for the ECM morsels. In embodiments, the human cell lines used as the source of the decellularized ECM morsels can be of different maturity, such as adult, juvenile, or fetal cells.

In embodiments, methods of making human ECM microtissues or spheroids are provided. In embodiments, disclosed methods comprise seeding cultured mammalian, for example human, cells into, for example, micro-wells using a non cell-adhesive micro-mold platform technology.

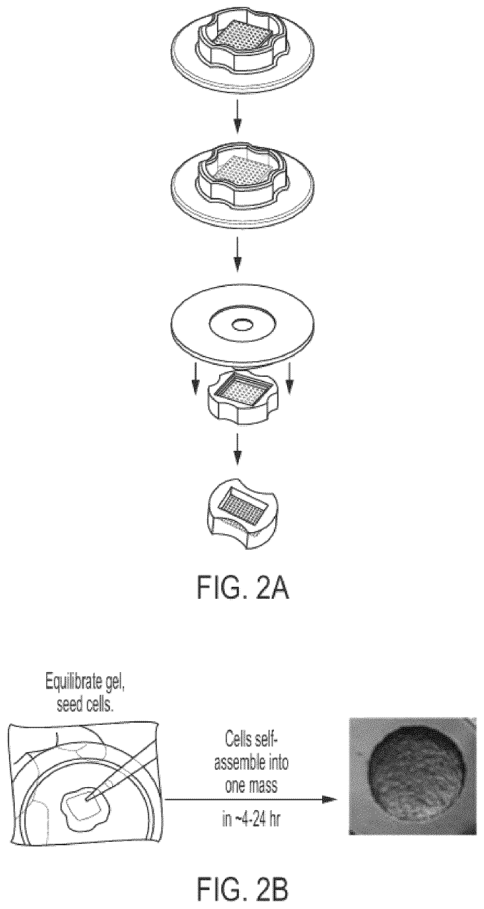

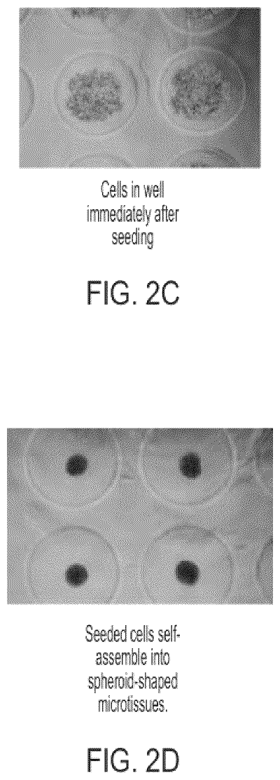

FIG. 2 illustrates a disclosed method for generating spheroid-shaped microtissues for fabricating human ECM morsels. The process begins by seeding cultured human cells into micro-wells using a non-adhesive micro-mold platform technology (FIGS. 2A and 2B). The seeded cells aggregate, synthesize, assemble, and deposit human ECM. Resulting ECM microtissues or spheroids (each approximately 50 to 300 μm in diameter) within each of the micro-wells (each approximately 400-800 μm in diameter) are illustrated in FIGS. 2C and 2D.

In embodiments, the micro-wells can be generated from any suitable material, such as agarose. For example, 2% agarose can be used to generate the micro-wells where the cells, such as living human cells, can aggregate synthesize, assemble, and deposit human ECM.

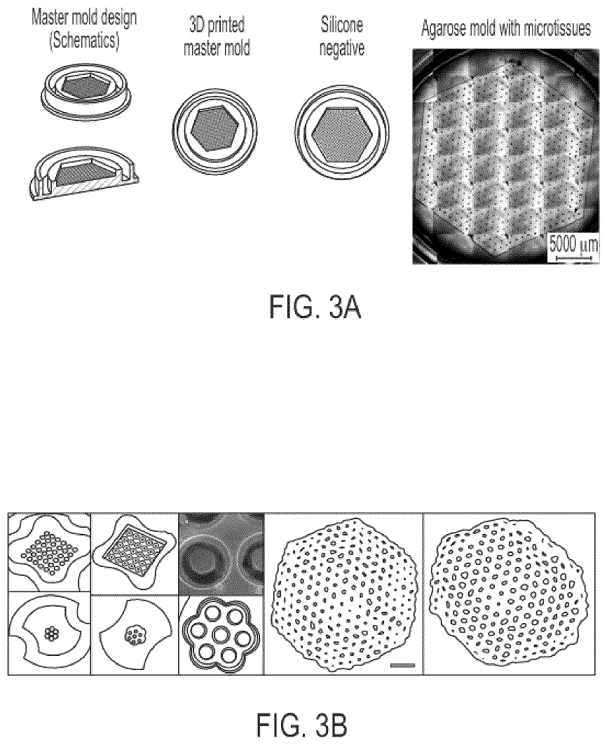

FIG. 3 illustrates an embodiment employing non-adhesive micro-mold platform technology, including schematics of a master mold (FIG. 3A), as well as the corresponding 3D printed mold, the silicone negative, and an image of an agarose mold with ECM microtissue within the micro-wells. FIG. 3B illustrates another embodiment, using ring- and honeycomb-shaped molds, the corresponding negative replicates in agarose, and the resulting ring- and honeycomb-shaped 3D human ECM microtissues (6 million cells, 2 cm across), removed from the agarose gel and stained for viable cells with calcein-AM. Also illustrated is a decellularized honeycomb-shaped 3D tissue of human dermal fibroblasts (14 million cells) showing ECM made after only 3 days; 2 cm across.

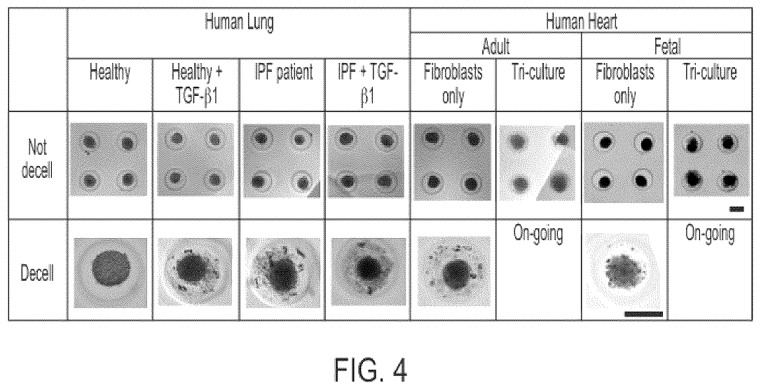

FIG. 4 illustrates a further method of generating decellularized human ECM morsels. In the embodiment of FIG. 4, fetal and adult human heart cells or adult lung cells are cultured and seeded into micro-wells to generate ECM microtissue or spheroids. The ECM spheroid microtissues can be decellularized inside the micro-wells. The resulting decellularized fetal and adult human heart or human lung ECM morsels maintain their spheroid shape following the decellularization procedure. Scale bar=200 μm.

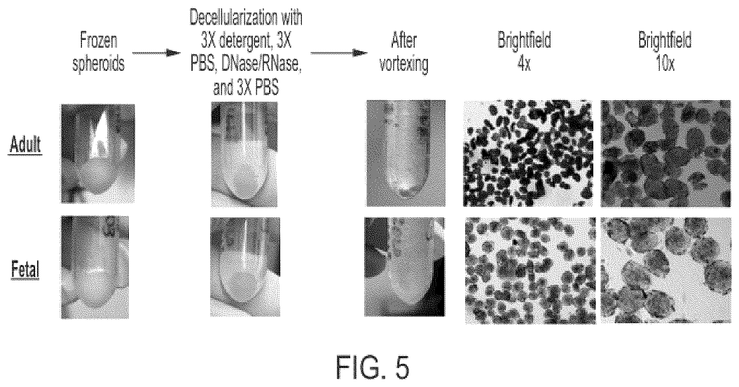

FIG. 5 illustrates an additional or alternative method of generating decellularized human ECM morsels. In FIG. 5, fetal and adult human heart cells can be cultured and seeded into micro-wells to generate ECM microtissue or spheroids. The ECM spheroid microtissues can be collected, decellularized, and mixed, for example vortexed. The resulting decellularized fetal and adult human heart ECM morsels maintain their spheroid shape following the decellularization procedure.

In embodiments, micro-molded, non-adhesive, cell aggregation devices can comprise a plurality of cell aggregation recesses in the shape of, for example, depressions or troughs. In embodiments, agarose can be employed as the hydrogel material and the cell aggregation recesses can be established, in embodiments, as follows. Troughs can be 400 μm wide with bottoms rounded with, for example, 200 μm radii. Disclosed embodiments can comprise rows of troughs of increasing length per gel. For example, in an embodiment, each row can have 11 troughs, two of which are 400 μm long, then one each of 600 μm through 1800 μm increasing at 200 μm lengths, then two 2200 μm troughs. In embodiments, tori-shaped recesses can be 800 μm deep, with circular track 400 μm wide. In embodiments, the recess bottom can comprise a radius of 200 μm.

In embodiments, it can take, for example, about 4 hours to about 24 hours for the cells to assemble into mammalian, for example human, ECM microtissues or spheroids and begin to generate the ECM (FIG. 2C). For example, in embodiments, cell assembly can take 4 hours, 6 hours, 8 hours, 10 hours, 12 hours, 14 hours, 16 hours, 18 hours, 20 hours, 22 hours, 24 hours, 26 hours, 28 hours, or the like.

In embodiments, cell assembly can take at least 4 hours, at least 6 hours, at least 8 hours, at least 10 hours, at least 12 hours, at least 14 hours, at least 16 hours, at least 18 hours, at least 20 hours, at least 22 hours, at least 24 hours, at least 26 hours, at least 28 hours, or the like.

Disclosed methods of generating the mammalian, for example human ECM microtissues or spheroids using the micro-mold technology provides for a stable, long-term, reproducible culture platform to form 3D human ECM microtissues or spheroids at high cell density (FIG. 2C). In addition, the micro-mold technology does not require that a scaffold material be used, thus, in embodiments, only the cultured cells, for example human cells, are needed to add to the micro-wells to generate spheroid-shaped microtissues. This approach allows for optimal cell-to-cell communication and movement, allowing mixtures of different cell types to interact while undergoing complex 3D morphological changes and differentiation. The micro-mold technology can allow cells in micro-wells to be cultured statically with exchange of cell culture medium that allows cell-secreted ECM to be concentrated at the site of secretion. In addition, the micro-molds can be customizable for a wide variety of tissue shapes and sizes based on by initial mold geometry (FIGS. 3A and 3B). Moreover, the mold geometry directs cellular alignment and organization, which subsequently affects the ECM microstructure and alignment, as well as bulk mechanical properties. Thus, unlike scaffold-based or cell-sheet methods, the micro-mold technology can be used to promote ECM alignment to better mimic the native tissue, which can increase therapeutic efficacy by promoting cell attachment and migration. The amount of time required for the seeded cells to sufficiently generate human ECM can be dependent on the type of cell, as well as the growing conditions. In one embodiment, seeded human cardiac fibroblasts can generate ECM in about 3 to about 12 days. For example, in embodiments, seeded human cardiac fibroblasts can generate ECM in about 3 days, 4 days, 5 days, 6 days, 7 days, 8 days, 9 days, 10 days, 11 days, 12 days, or the like.

In embodiments, the human ECM microtissues can then be collected and decellularized, or decellularized directly within the micro-molds. Decellularization can be accomplished by treatment with a mild detergent followed by a treatment to remove DNA (FIG. 3). Disclosed ECM microtissues can be collected from the micro-wells by, for example, pipetting, subjected to an optional freeze-thaw step, treatment with salt solutions and a mild detergent, followed by treatment with the enzymes DNase and RNase can be used to remove the DNA and RNA from the human microtissues.

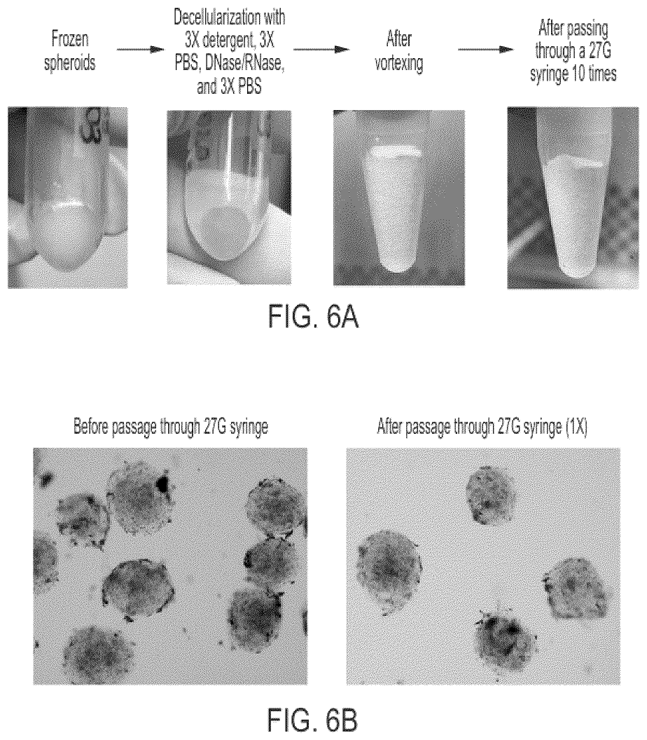

In embodiments, successful decellularization kills the cells, removes most of the cellular material, and removes or destroys most of the DNA, leaving behind human ECM in tissue and age-specific three-dimensional architecture and mechanical stiffness. The resulting composition, comprising decellularized human ECM morsels or small porous spheres of human ECM, can freely pass through a small diameter hypodermic needle (FIG. 4) and can therefore be easily injected into organs or tissues in need of tissue repair or regeneration. Disclosed decellularized human ECM morsels can be evaluated for the presence of DNA using techniques know to a person skilled in the art. DNA-free decellularized ECM can undergo constructive remodeling in-vivo with minimal adverse effects. FIG. 6 illustrates decellularized human fetal ECM morsels before and after passage through a 27G syringe 10 times.

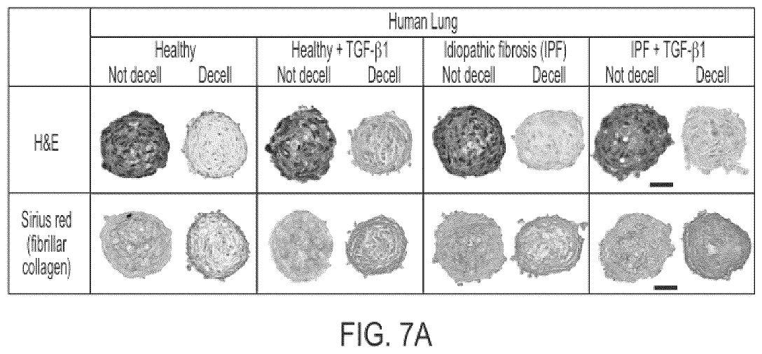

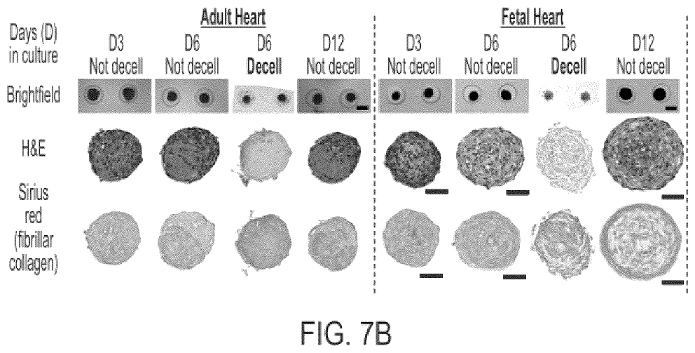

FIG. 7A illustrates one embodiment comprising stained ECM spheroid microtissues from human lung cells, both healthy cells and cells from a patient with idiopathic pulmonary fibrosis (IPF), and FIG. 7B illustrates ECM spheroid microtissues from adult and fetal human heart cells (fibroblasts alone, or co-cultured with cardiac myocytes and microvascular endothelial cells). Fibroblast-only adult or fetal heart ECM microtissues were cultured for 3, 6, 9 or 12 days, whereas the tri-cultures were only cultured for 6 days. Images of the ECM spheroid microtissues in brightfield, stained with hematoxylin and eosin (H&E) or SIRIUS RED™, before decellularization are illustrated and ECM spheroid microtissues from adult and fetal human heart or adult lung cells after decellularization are also illustrated. Scale bars=50 μm.

In accordance with another non-limiting aspect of the present disclosure, which may be used in combination with each or any of the above-mentioned aspects, the composition of the decellularized mammalian, for example human, ECM morsels can be designed for a particular patient in need thereof. In embodiments, the composition of the decellularized human ECM morsels can be generated by selecting a desirable cell type or selecting a combination or mixture of different cell types to generate decellularized human ECM morsels. Starting cell types may include, but are not limited to the following: cardiac fibroblasts, cardiac myocytes and cardiac microvascular endothelial cells could be used in combination to generate a decellularized human ECM that may be administered to a particular patient in need thereof. These “designer” ECM compositions comprise unique compositions and potencies that do not exist in native tissues or whole organs, thus providing the practitioner the ability to design and produce compositions particularly suited for a desired treatment in a specific patient.

In addition to selecting different cell types or combining different cell types to form the starting ECM microtissues, as previously described, “designer” ECM compositions can also be generated by treating the starting 3D ECM microtissues with growth factors and/or drugs that can alter and/or improve the production of ECM and its quality. In some embodiments, additives to the culture media such as growth factors, cytokines and drugs can influence the amounts and types of ECM produced by cells. By incubating the microtissues with anti-inflammatory mediators such as, for example, interleukin 4, interleukin 10, interleukin 11 or interleukin 13 can influence the microtissues to produce an “anti-inflammatory designer” ECM. Further “designer” ECM compositions can comprise ECM produced from recombinant cells, for example recombinant cells producing a cytokine.

Additionally or alternatively, as previously explained, the desired size (i.e., diameter) and/or shape of the decellularized human ECM morsels can be changed or adjusted via the number of cells seeded into the micro-molds, the geometry of the initial micro-mold, or the length of culture time for ECM morsels made with human cells. A particular micro-mold could be used to generate 3D ECM spheroid microtissues of a precise size can generate decellularized human ECM morsels that can pass through the desired need size during administration.

In accordance with another non-limiting aspect of the present disclosure, a method of generating decellularized human ECM morsels from cultured cells can be performed using an automated process.

The presently disclosed methods of generating decellularized human ECM morsels from cultured cells for use in tissue repair and regeneration can result in a purer ECM composition, because the starting material is generally more highly defined with no fat, fascia or connective tissues, bacteria, and/or other materials that can contaminate whole organs. There can also be less batch-to-batch variability of ECM compositions derived from cultured cells rather than whole organs. In addition, the decellularized human ECM morsels derived from cultured cells require fewer steps and a gentler process that can preserve function as compared to decellularized human ECM morsels derived from whole organs or from cultured cell sheets. The disclosed processes for producing decellularized human ECM morsels eliminate the steps of lyophilization, digestion, and reconstitution which are typically more laborious, costly and most importantly, disrupt the structure of ECM and its potency.

ECM Compositions-Methods of Use: Disclosed herein are methods of using disclosed, customized decellularized human ECM compositions. For example, in embodiments, disclosed methods of use comprise treatment of, for example, heart disease.

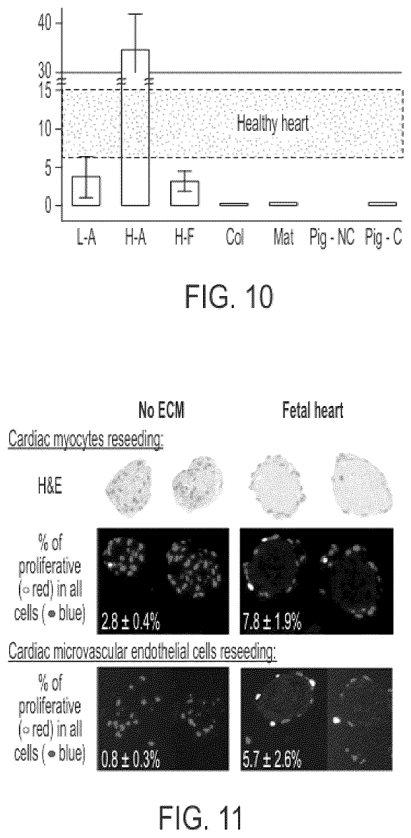

It has been shown that the structural, biochemical and mechanical cues of ECM can facilitate cell attachment, migration and signalling, all of which are critical for tissue regeneration and repair. It is hypothesized that a stiffness similar to healthy myocardium would be ideal for cardiac tissue engineering. FIG. 10 shows that decellularized fetal human heart ECM morsels had comparable mechanical stiffness (elastic modulus) as healthy heart tissue, thus providing an ideal mechanical property for use in heart treatments.

In one aspect, disclosed herein, are methods of using the decellularized human ECM morsels as treatments for the purposes of tissue repair, tissue regeneration, and tissue augmentation. The decellularized human ECM composition may be formulated as an injectable, as a patch, and/or as an aerosol. In embodiments comprising a patch substrate, the patch can comprise a biodegradable material, i.e. it is naturally absorbed by the patient's body after some time. In embodiments, the biodegradable material is biocompatible, i.e. have no harming effect to the patient to whom the material is administered. Thus, the biocompatible matrix can be a biomaterial selected from biopolymers such as a proteins or polysaccharides, for example a biomaterial such as collagen, gelatin, fibrin, a polysaccharide, e.g. hyaluronic acids, chitosan, and derivatives thereof, collagen, chitosan, etc.

In another embodiment, customized decellularized mammalian, for example human, ECM morsels can be injected into the skin to achieve augmentation as a strategy for tissue repair as well as for cosmetic applications and treatments, such as, for example, treatment of the lip, treatment of the cheek, treatment of the forehead, dermal filler treatments, or the like. For example, in an embodiment, a disclosed EOM composition comprising hyaluronic acid can be administered to a subject's lips to add fullness.

Disclosed embodiments can comprise treatment to reduce the effects of aging upon tissues such as skin. For example, over time, various body structures lose function in an unpredictable sequence. ECM provides a commonality amongst these intricate processes, and thus disclosed methods can comprise treatment to reduce the effect of age upon the skin.

Furthermore, in another embodiment, decellularized human ECM morsels can be applied topically to aid in wound healing, for example as a solution, gel, or patch.

In accordance with another non-limiting aspect of the present disclosure, which may be used in combination with the other aspects, compositions of decellularized human ECM morsels can be mixed with stem cells and used as cell carriers for the safe transplantation of administered cells, or mixed with therapeutic compounds/drugs as a delivery agent, such as via injection are described.

Also disclosed herein is the ability of administered decellularized human ECM morsels to promote cell viability, cell proliferation, cell migration, chemotaxis, and/or capillary tube formation in vivo.

It should be understood that various changes and modifications to the embodiments described herein will be apparent to those skilled in the art. Such changes and modifications can be made without departing from the spirit and scope of the present subject matter and without diminishing its intended advantages. It is therefore intended that such changes and modifications be covered by the appended claims.

As various changes could be made in the above-described sources and methods without departing from the scope of the invention, it is intended that all matter contained in the above description and in the examples given below, shall be interpreted as illustrative and not in a limiting sense.

### Decellularized Human Mesenchymal Stem Cell-Derived Extracellular Matrix for the Treatment of Heart Failure

The present disclosure, in part, relates to novel sources, compositions, and methods of preparing and using decellularized mammalian ECM particles. Generally, the methods and compositions disclosed herein relate to the use of human ECM particles for the purpose of heart tissue repair and regeneration but can be used for any repair to save lives.

Human mesenchymal stem cells (MSCs) derived from human umbilical cords are a potentially a promising source for entirely human ECM particles because they are stem cells and are a cell source well known to the pharmaceutical industry, which has already figured out how to scale up the production of these cells in 2D for testing them in as a living cell therapy in clinical trials. Disclosed embodiments comprise three-dimensional (3D) in vitro MSC cell culture systems to engineer specifically tailored microtissues suitable for decellularization to make a unique formulation of the human ECM, a formulation that cannot be found or extracted from cadaver or animal sources.

MSC tissues can be decellularized to fabricate decellularized human ECM, for example in the form of small porous particles that are equal to or less than 800 μm in diameter, thus providing “flowable” compositions that can be administered with, for example, a cannula such as a needle. Disclosed compositions can be administered via injection due to the size and shape of the ECM particles.

The resulting decellularized human ECM particles can support heart tissue regeneration and are herein shown to have beneficial effects as measured by an increase in fractional shortening, a decrease in infarct scar size and an increase in capillaries/arterioles.

Disclosed herein are customized decellularized human ECM compositions. Disclosed compositions are prepared from xeno-free human umbilical cord-derived cultured human MSCs.

Disclosed herein are methods for producing customized decellularized human ECM compositions. MSCs are grown in tissue culture flasks and released from the flasks by trypsinization. The cells are counted and seeded onto micro-molded agarose gels cast from a mold designed to scale up the process. Prior to seeding the cells, the agarose is equilibrated with a selected cell culture medium. The MSCs form microtissues and 7 to 10 days later the MSC microtissues are decellularized by treatment with a mild detergent (e.g., Triton X-100) followed by the enzyme DNase to remove DNA. This results in the production of MSC ECM particles. The ECM particles are made using a highly controlled industrial aseptic process that helps assure consistency and safety, of utmost importance to the FDA. The matrisome of batches of MSC ECM particles having acceptably low levels of DNA were analyzed by proteomics and shown to be highly consistent from batch to batch.

Disclosed herein are methods of using customized decellularized human MSC ECM compositions. For example, disclosed methods of use comprise repair and regeneration of a cardiovascular injury in a human. In some embodiments, disclosed methods of use can comprise administration of a disclosed MSC ECM composition to a treatment area in the heart. In some embodiments, disclosed compositions can be administered via injection. In some embodiments, disclosed flowable compositions can be administered using a cannula such as a needle, for example a syringe. In some embodiments, the MSC ESM particles can have a wide variety of applications in the field of regenerative medicine where scarring or fibrosis is problematic. Other implementations are also described and recited herein.

### Biopharmaceutical Definitions

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, sumoylated, farnesylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions, insertions, or suppressor mutations to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions, substitutions, or suppressor mutations. Variant polypeptide-encoding DNA sequences encompass sequences that comprise one or more insertions, deletions, or substitutions of nucleotides when compared to a native or reference DNA sequence, but that encode a variant protein or fragment thereof that retains activity or function. A wide variety of PCR-based site-directed mutagenesis approaches are known in the art and can be applied by the ordinarily skilled artisan.

As used herein, the term “nucleic acid” or “nucleic acid sequence” refers to any molecule, preferably a polymeric molecule, incorporating units of ribonucleic acid, deoxyribonucleic acid or artificial nucleic acid analogues (e.g., peptide nucleic acid, morpholino- and locked nucleic acid, glycol nucleic acid, threose nucleic acid and hexitol nucleic acid), or any analogs thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell is typically still referred to as “engineered” even though the actual manipulation was performed on a prior entity.

In some embodiments, a nucleic acid encoding a polypeptide as described herein (e.g., an antibody or antibody reagent) is comprised by a vector. In some of the aspects described herein, a nucleic acid sequence encoding a given polypeptide as described herein, or any module thereof, is operably linked to a vector. A vector can include, but is not limited to, a cloning vector, an expression vector, a plasmid, phage, transposon, cosmid, chromosome, virus, oncolytic virus-like vesicle (VLV), virion, extracellular vesicles, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA, peptides and proteins and as appropriate, secreting peptides or proteins, including where applicable, but not limited to, for example, transcription, transcript processing, translation and protein folding, modification, degradation, shuttling, shuffling, and processing. “Expression products” include RNA transcribed from a gene, and polypeptides obtained by translation of mRNA transcribed from a gene. The term “gene” means the nucleic acid sequence which is transcribed (DNA) to RNA in vitro, ex vivo or in vivo when operably linked to appropriate regulatory sequences. The gene may or may not include regions preceding and following the coding region, e.g., 5′ untranslated (5′UTR) or “leader” sequences and 3′ UTR, “trailer” or “leader” sequences, as well as intervening sequences (introns) or spacers between individual coding segments (exons).

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell or secreted in the case of secreted polypeptides. A chemically synthesized nucleic acid or polypeptide or one synthesized using in vitro transcription/translation is considered “isolated.” The terms “purified” or “substantially purified” refer to an isolated nucleic acid or polypeptide that is at least 95% by weight the subject nucleic acid or polypeptide, including, for example, at least 96%, at least 97%, at least 98%, at least 99% or more. In some embodiments, the antibody, antigen-binding portion (e.g., scFv or nanobody) thereof, bispecific or tri-specific T cell engager, bispecific or tri-specific NK cell engager, immune cell recruiter, or chimeric antigen receptor (CAR) described herein is isolated. In some embodiments, the antibody, antibody reagent, antigen-binding portion thereof, engager, recruiter or CAR described herein is purified.

As used herein, “engineered” refers to the aspect of having been manipulated by the hand of man. For example, an antibody, antibody reagent, antigen-binding portion (e.g., scFv or nanobody) thereof, or tri-specific T cell engager, bispecific or tri-specific NK cell engager, immune cell recruiter (such as Bifunctional checkpoint-inhibitory T cell engager (CiTE), simultaneous multiple interaction T cell engager (SMITE), immune-mobilizing monoclonal TCRs against cancer (ImmTACs)), CAR or is considered to be “engineered” when the sequence of the antibody, antibody reagent, antigen-binding portion thereof, engager, recruiter or CAR is manipulated by the hand of man to differ from the sequence of an antibody as it exists in nature. As is common practice and is understood by those in the art, progeny and copies of an engineered polynucleotide and/or polypeptide are typically still referred to as “engineered” even though the actual manipulation was performed on a prior entity.

Other terms are defined herein within the description of the various aspects of the invention.

Various non-exhaustive, non-limiting aspects of compositions according to the present disclosure may be useful alone or in combination with one or more other aspects described herein. Disclosed systems, compositions and methods provide unique advantages to both patients and practitioners. For example, disclosed embodiments can produce customized 3D MSC microtissues. In some embodiments, these MSC microtissues can be used to fabricate decellularized human ECM particles.

The biochemical/chemical composition (both biochemical/chemical species and/or their contents), collagen architecture, and mechanical properties of the ECM particles generated by the mammalian cells in microtissues are uniquely dependent on the types of mammalian cells used (tissue origin, age, disease state, etc.). Thus, particular cells can be employed to achieve desired ECM properties.

The biochemical/chemical composition (both biochemical/chemical species and/or their contents), collagen architecture, and mechanical properties of the ECM particles generated by the mammalian cells in microtissues are further dependent on the culturing conditions of the mammalian cells as they form microtissues. For example, cell culture media composition, culturing time, oxygen level, and the presence or amount of additional biological factors including and not limited to growth factors, cytokines, drugs, and the like can be adjusted to produce the desired ECM properties.

The biochemical/chemical composition (both biochemical/chemical species and/or their contents), collagen architecture, and mechanical properties of the disclosed ECM particles generated by the mammalian cells in microtissues can be different from ECM extracted from mammalian tissues found in nature (for example, pig heart, human heart, etc.).

Disclosed ECM particles can form flowable compositions that can pass through a syringe with an attached needle, where the needle inner diameter (ID) would depend on the size of the ECM particles. For example, ECM particles with a diameter of ˜200 μm will be able to pass through any needle with an ID equal or larger than the ID of a 27 gauge (G) needle (ID=200 μm).

Disclosed ECM particles can be made using fewer steps than ECM extracted from animal or human tissues and organs. Disclosed ECM particles can be made using aseptic conditions.

Disclosed ECM particles are biocompatible in vitro, such that mammalian cells placed on the decellularized mammalian ECM particles can survive and multiply.

Different ECM characteristics (including, for example, physical properties, biochemical/chemical compositions, collagen architecture, mechanical properties, and combinations thereof) are likely to have different effects on surrounding tissue once implanted in vivo, allowing for customized ECM to be developed for any number of clinical conditions, especially in regenerative medicine.

### ECM Compositions

Disclosed herein are customized decellularized compositions, particularly decellularized human MSC ECM compositions, whose properties can be specifically tailored to suit particular patients (or patient groups) as well as particular indications. The use of foreign, non-human decellularized ECM for tissue regeneration and/or repair can be prone to causing immune reactions in the subject. Decellularized human ECM particles overcome the issue of immune reactions because the ECM is human. Thus, in some embodiments, the decellularized cells are human MSC cells.

In some embodiments, disclosed compositions are derived from cultured human MSC cells used to form decellularized human ECM particles. The ECM can comprise a complex 3D architecture of structural proteins such as collagens, glycoproteins, proteoglycans, ECM regulators, secreted factors and ECM affiliated proteins. In some embodiments, the ECM provides structural support, as well as signals for tissue regeneration.

### ECM Compositions—Methods of Production

Disclosed herein are methods of producing customized decellularized human MSC-derived ECM compositions with desired physical or chemical properties. For example, in some embodiments, disclosed methods are derived from cultured human cells.

Disclosed methods can comprise: 1. Growing xeno-free human umbilical cord-derived Mesenchymal/Stromal cells (MSCs) in tissue culture flasks. 2. Releasing the cells from the flasks (e.g., by trypsinization). 3. Counting the cells and seeding them onto micro-molded agarose gels cast from a mold and equilibrated with a selected cell culture medium. 4. Allowing the MSC cells to form 3D microtissues for 7 to 10 days. 5. Collecting the microtissues and decellularizing the microtissues by treatment with a mild detergent (e.g., Triton X-100) followed by the enzyme DNase to remove DNA to form ECM particles.

In some embodiments, methods of making human ECM microtissues or spheroids are provided. In some embodiments, disclosed methods comprise seeding cultured mammalian, for example human, cells into, for example, micro-wells using a non-cell-adhesive micro-mold platform technology.

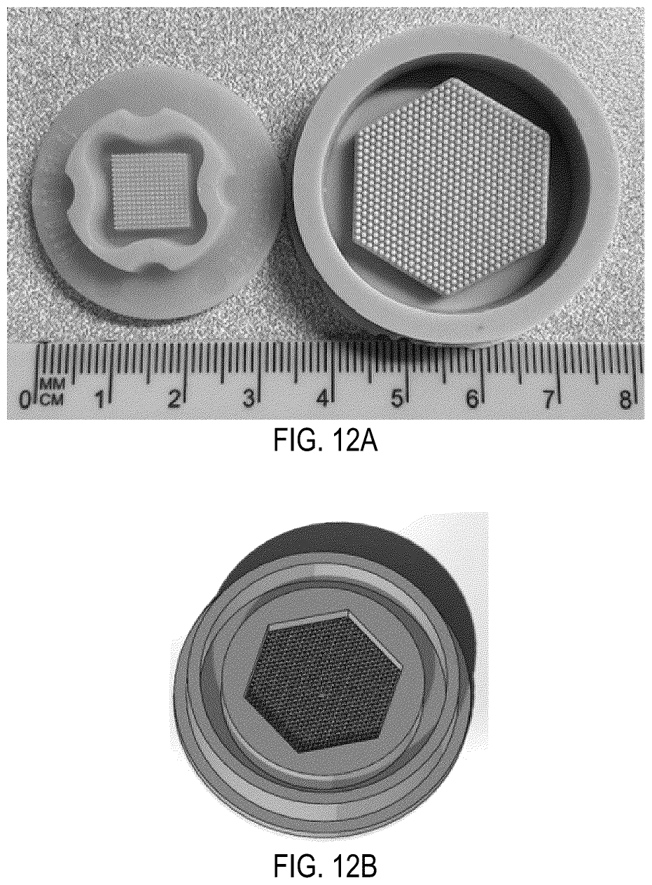

FIGS. 12A-12B illustrate molds for generating spheroid-shaped MSC microtissues for fabricating human ECM particles. The process begins by seeding cultured human cells into micro-wells using a non-adhesive micro-mold platform technology. The seeded cells aggregate, synthesize, assemble, and deposit human ECM. Resulting MSC microtissues or spheroids are each approximately 50 to 300 μm in diameter within each of the micro-wells (each approximately 400-800 μm in diameter). In some embodiments, the micro-wells can be generated from any suitable material, such as agarose. For example, 2% agarose can be used to generate the micro-wells where the cells, such as living human cells, can aggregate synthesize, assemble, and deposit human ECM.

In some embodiments, micro-molded, non-adhesive, cell aggregation devices can comprise a plurality of cell aggregation recesses in the shape of, for example, depressions or troughs. In some embodiments, agarose can be employed as the hydrogel material and the cell aggregation recesses can be established, in some embodiments, as follows. Troughs can be 400 μm wide with bottoms rounded with, for example, 200 μm radii. Disclosed embodiments can comprise rows of troughs of increasing length per gel. For example, in an embodiment, each row can have 11 troughs, two of which are 400 μm long, then one each of 600 μm through 1800 μm increasing at 200 μm lengths, then two 2200 μm troughs. In some embodiments, tori-shaped recesses can be 800 μm deep, with circular track 400 μm wide. In some embodiments, the recess bottom can comprise a radius of 200 μm.

Disclosed methods of generating the human MSC microtissues or spheroids using the micro-mold technology provides for a stable, long-term, reproducible culture platform to form 3D human MSC microtissues or spheroids at high cell density. In addition, the micro-mold technology does not require that a scaffold material be used, thus, in some embodiments, only the cultured human MSC cells are needed to add to the micro-wells to generate spheroid-shaped MSC microtissues.

In some embodiments, the human MSC microtissues can be collected and decellularized, or decellularized directly within the micro-molds. Decellularization can be accomplished by treatment with a mild detergent followed by a treatment to remove DNA. Disclosed MSC microtissues can be collected from the micro-wells by, for example, pipetting, subjected to an optional freeze-thaw step, treatment with salt solutions and a mild detergent, followed by treatment with the enzyme DNase and optionally RNase to remove the DNA and RNA from the human MSC microtissues.

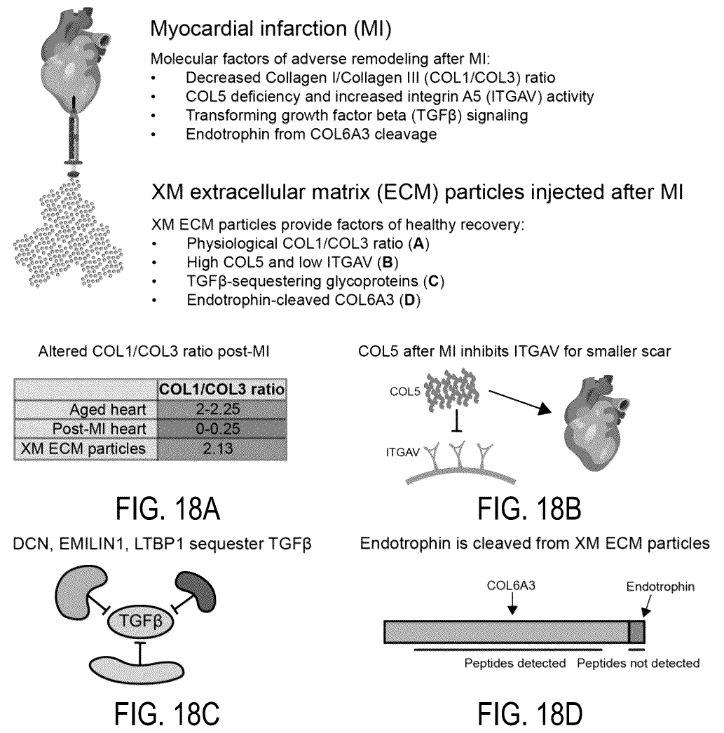

In some embodiments, successful decellularization kills the cells, removes most of the cellular material, and removes or destroys most of the DNA, leaving behind human ECM. The resulting composition, comprising decellularized human ECM particles or small porous spheres of human ECM, can freely pass through a small diameter hypodermic needle (FIGS. 18A-18D) and can therefore be easily injected into organs or tissues in need of tissue repair or regeneration.

“Designer” ECM compositions can be generated by treating the starting 3D MSC microtissues with growth factors and/or drugs that can alter and/or improve the production of ECM and its quality. In some embodiments, additives to the culture media such as growth factors, cytokines and drugs can influence the amounts and types of ECM produced by cells. By incubating the microtissues with anti-inflammatory mediators such as, for example, interleukin 4, interleukin 10, interleukin 11 or interleukin 13 can influence the microtissues to produce an “anti-inflammatory designer” ECM.

Additionally, or alternatively, as previously explained, the desired size (i.e., diameter) and/or shape of the human ECM particles can be changed or adjusted via the number of cells seeded into the micro-molds, the geometry of the initial micro-mold, or the length of culture time for ECM particles made with human MSC cells. A particular micro-mold could be used to generate 3D MSC spheroid microtissues of a precise size that can generate human ECM particles that can pass through the desired need size during administration.

In accordance with another non-limiting aspect of the present disclosure, a method of generating human ECM particles from cultured human MSC cells can be performed using an automated process.

The presently disclosed methods of generating human ECM particles from cultured MSC cells for use in tissue repair and regeneration can result in a purer ECM composition, because the starting material is more highly defined with no fat, fascia or connective tissues, bacteria, and/or other materials that can contaminate whole organs. There can also be less batch-to-batch variability of ECM compositions derived from cultured MSC cells rather than whole organs. In addition, the human ECM particles derived from cultured cells require fewer steps and a gentler process that can preserve function as compared to decellularized human ECM particles derived from whole organs or from cultured cell sheets. The disclosed processes for producing decellularized human ECM particles eliminate the steps of lyophilization, digestion, and reconstitution which are typically more laborious, costly and most importantly, disrupt the structure of ECM and its potency.

### ECM Compositions—Methods of Use

Disclosed herein are methods of using disclosed, customized decellularized human ECM compositions. For example, in some embodiments, disclosed methods of use comprise treatment of, for example, heart disease.

It has been shown that the structural, biochemical and mechanical cues of ECM can facilitate cell attachment, migration and signaling, all of which are critical for tissue regeneration and repair. It is hypothesized that a stiffness similar to healthy myocardium would be ideal for cardiac tissue engineering. We previously established that decellularized fetal human heart ECM particles had comparable mechanical stiffness (elastic modulus) as healthy heart tissue, thus providing an ideal mechanical property for use in heart treatments. See, FIG. 10 in PCT Application WO 2022/140530.

In one aspect, disclosed herein, are methods of using the decellularized human ECM particles as treatments for the purposes of tissue repair, tissue regeneration, and tissue augmentation. The decellularized human ECM composition may be formulated as an injectable, as a patch, and/or as an aerosol. In some embodiments comprising a patch substrate, the patch can comprise a biodegradable material, i.e., it is naturally absorbed by the patient's body after some time. In some embodiments, the biodegradable material is biocompatible, i.e., have no harming effect to the patient to whom the material is administered. Thus, the biocompatible matrix can be a biomaterial selected from biopolymers such as proteins or polysaccharides, for example a biomaterial such as collagen, gelatin, fibrin, a polysaccharide, e.g., hyaluronic acids, chitosan, and derivatives thereof, collagen, chitosan, etc.

In another embodiment, customized decellularized mammalian, for example human, ECM particles can be injected into the skin to achieve augmentation as a strategy for tissue repair as well as for cosmetic applications and treatments, such as, for example, treatment of the lip, treatment of the cheek, treatment of the forehead, dermal filler treatments, or the like. For example, in an embodiment, a disclosed ECM composition comprising hyaluronic acid can be administered to a subject's lips to add fullness.

Disclosed embodiments can comprise treatment to reduce the effects of aging upon tissues such as skin. For example, over time, various body structures lose function in an unpredictable sequence. ECM provides a commonality amongst these intricate processes, and thus disclosed methods can comprise treatment to reduce the effect of age upon the skin.

Furthermore, in another embodiment, decellularized human ECM particles can be applied topically to aid in wound healing, for example as a solution, gel, or patch.

In accordance with another non-limiting aspect of the present disclosure, which may be used in combination with the other aspects, compositions of decellularized human ECM particles can be mixed with stem cells and used as cell carriers for the safe transplantation of administered cells, or mixed with therapeutic compounds/drugs as a delivery agent, such as via injection are described.

Also disclosed herein is the ability of administered decellularized human ECM particles to promote cell viability, cell proliferation, cell migration, chemotaxis, and/or capillary tube formation in vivo.

In some embodiments, the techniques described herein relate to a customized flowable composition including decellularized human heart extracellular matrix (ECM), wherein said customized flowable composition includes ECM including a desired physical or biochemical/chemical profile differing from the physical or biochemical/chemical profile of in-vivo derived adult human heart ECM in collagen and sulphated glycosaminoglycans (sGAG) content.

According to some aspects, the techniques described herein relate to a method of producing customized decellularized human heart extracellular matrix (ECM) morsels, the method including: seeding cultured human heart cells into micro-wells, wherein the cultured human heart cells generate an ECM microtissue included of the cultured cells and ECM produced from the cultured cells; collecting the ECM microtissue as spheroid particles; and decellularizing the ECM microtissue; wherein said customized decellularized ECM morsels include a desired physical or biochemical/chemical profile differing from the physical or biochemical/chemical profile of in-vivo derived adult human heart ECM in collagen and sulphated glycosaminoglycans (sGAG) content.

In some embodiments, the techniques described herein relate to a method, wherein said decellularizing includes mixing the microtissue with at least one of a detergent, a buffer, a DNAse, or an RNase.

In some aspects, the techniques described herein relate to a method, wherein said ECM microtissue includes spherical particles having a diameter of less than or equal to 800 μm.

In some embodiments, the techniques described herein relate to a method, wherein said cells include recombinant human cells.

According to some aspects, the techniques described herein relate to a method, wherein said cells include at least one of cardiac fibroblasts, cardiac myocytes and cardiac microvascular endothelial cells.

In some embodiments, the techniques described herein relate to a customized flowable composition including decellularized human heart extracellular matrix (ECM) made by the method including: seeding cultured human heart cells into micro-wells, wherein the cultured cells generate an ECM microtissue included of the cultured cells and an ECM; collecting the ECM microtissue as spheroid particles; and decellularizing the ECM microtissue; wherein said customized flowable composition includes ECM including a desired physical or biochemical/chemical profile differing from the physical or biochemical/chemical profile of in-vivo derived adult human heart ECM in collagen and sulphated glycosaminoglycans (sGAG) content.

In some aspects, the techniques described herein relate to a composition, wherein said decellularizing includes mixing the cells with at least one of a detergent, a buffer, a DNAse, or an RNase.

In some embodiments, the techniques described herein relate to a composition, wherein said method further includes passing the mixture through a syringe.

According to some aspects, the techniques described herein relate to a composition, wherein said syringe includes a 27 G needle.

In some embodiments, the techniques described herein relate to a composition, wherein said cells include recombinant human cells.

In some aspects, the techniques described herein relate to a composition, wherein said cells include at least one of cardiac fibroblasts, cardiac myocytes and cardiac microvascular endothelial cells.

In some embodiments, the techniques described herein relate to a method, wherein said micro-wells are scaffold-free.

According to some aspects, the techniques described herein relate to a composition, wherein said micro-wells are scaffold-free.

In some embodiments, the techniques described herein relate to a method, wherein said modifying includes adjusting at least one of cell culture media composition, culturing time, oxygen level, or the presence or amount of additional biological factors.

In some aspects, the techniques described herein relate to a method, wherein said additional biological factors include at least one of a growth factor, a cytokine, and a drug.

In some embodiments, the techniques described herein relate to a method of producing customized decellularized human heart extracellular matrix (ECM) morsels, the method including: seeding cultured human heart cells into micro-wells, wherein the cultured cells generate an ECM microtissue included of the cultured cells and an ECM, wherein said ECM microtissue includes spherical particles having a diameter of less than or equal to 800 μm; collecting the ECM microtissue; and decellularizing the ECM microtissue; wherein said customized decellularized human heart ECM morsels include a desired physical or biochemical/chemical profile differing from the physical or biochemical/chemical profile of in-vivo derived adult human heart ECM in collagen and sulphated glycosaminoglycans (sGAG) content.

According to some aspects, the techniques described herein relate to a customized flowable composition, wherein the mechanical stiffness of the ECM is comparable to healthy human heart tissue.

In some embodiments, the techniques described herein relate to a method, wherein the mechanical stiffness of the ECM is comparable to healthy human heart tissue.

In some aspects, the techniques described herein relate to a customized flowable composition, wherein the mechanical stiffness of the ECM is comparable to healthy human heart tissue.

In some embodiments, the techniques described herein relate to a method, wherein the mechanical stiffness of the ECM is comparable to healthy human heart tissue.

According to some aspects, the techniques described herein relate to a customized flowable composition including decellularized mesenchymal stem cell extracellular matrix (MSC ECM), wherein said customized flowable composition includes ECM including a desired physical or biochemical/chemical profile differing from the physical or biochemical/chemical profile of non-MSC derived ECM in at least one of a physical property, a biochemical/chemical property, a mechanical property, or collagen architecture.

In some embodiments, the techniques described herein relate to a customized flowable composition, wherein said desired physical or biochemical/chemical profile differing from the physical or biochemical/chemical profile of in vivo derived ECM includes a different physical property.

In some aspects, the techniques described herein relate to a customized flowable composition, wherein said desired physical or biochemical/chemical profile differing from the physical or biochemical/chemical profile of in vivo derived ECM includes a different biochemical/chemical property.

In some embodiments, the techniques described herein relate to a customized flowable composition, wherein said desired physical or biochemical/chemical profile differing from the physical or biochemical/chemical profile of in vivo derived ECM includes a different mechanical property.

According to some aspects, the techniques described herein relate to a customized flowable composition, wherein said desired physical or biochemical/chemical profile differing from the physical or biochemical/chemical profile of in vivo derived ECM includes different collagen architecture.

In some embodiments, the techniques described herein relate to a method of producing customized decellularized human MSC extracellular matrix (MSC ECM) particles, the method including: (i) seeding cultured human MSC cells into medium-conditioned agarose gels in micro-wells, wherein the cultured MSC cells generate an MSC microtissue included of the cultured MSC cells; (ii) collecting the MSC microtissue; and (iii) decellularizing the MSC microtissue; wherein said customized decellularized MSC ECM particles include a desired physical or biochemical/chemical profile differing from the physical or biochemical/chemical profile of non-MSC-derived ECM in at least one of a physical property, a biochemical/chemical property, a mechanical property, or collagen architecture.

In some aspects, the techniques described herein relate to a method, wherein said decellularizing includes mixing the microtissue with at least one of a detergent, a buffer, a DNAse, or an RNase.

In some embodiments, the techniques described herein relate to a method, wherein said MSC ECM particles include spheroidal particles.

According to some aspects, the techniques described herein relate to a method, wherein said MSC ECM particles include spheroidal particles having a diameter of less than or equal to 800 um.

In some embodiments, the techniques described herein relate to a method of treating a cardiovascular injury, the method including administering a decellularized human MSC ECM composition to the injury area.

In some aspects, the techniques described herein relate to a method, wherein said cardiovascular injury is the result of a myocardial infarction.

In some embodiments, the techniques described herein relate to a method, wherein said micro-wells are scaffold-free.

According to some aspects, the techniques described herein relate to a method, wherein said additional biological factors include at least one of a growth factor, a cytokine, and a drug.

In some embodiments, the disclosure provides a method that may alter the proteome of a diseased heart, organ, and/or tissue by contacting it with extra-cellular matrix (ECM) particles. These ECM particles can be produced from decellularized 3D microtissues derived from human mesenchymal stem cells. The method may restore the normal ratio of different collagens in the heart, which can promote proper cardiac function and prevent fibrosis. Additionally, the method may alter the immune response in the heart by modulating the activity of macrophages, T cells, and B cells, potentially reducing inflammation and promoting tissue repair. The method may also enhance metabolism in the heart by improving mitochondrial function, increasing ATP production, and reducing oxidative stress, thereby enhancing the energy supply to cardiac tissue. Furthermore, the method may mitigate the effects of TGF-Beta, a growth factor associated with fibrosis, by reducing the activation of fibroblasts and the deposition of excessive extracellular matrix components. The ECM particles may be treated to improve crosslinking, stability, injectability, or compatibility with a patient, ensuring effective treatment of the diseased heart.

The process may involve the derivation of 3D microtissues from human mesenchymal stem cells, which can serve as a source for the extra-cellular matrix (ECM) particles. These ECM particles may be produced from decellularized 3D microtissues, potentially altering the proteome of a diseased heart. The contacting of the diseased heart with these ECM particles may facilitate the alteration of the proteome, which can be a step in the treatment process. The ECM particles may retain bioactive components and structural properties of the native tissue, which can be for their function. The alteration of the proteome may lead to various beneficial outcomes, such as restoring the normal ratio of collagen levels, altering immune responses, and improving metabolism. The ECM particles may also mitigate the effects of TGF-Beta, a growth factor associated with fibrosis, thereby reducing the activation of fibroblasts and the deposition of excessive extracellular matrix components. The method may be designed to change the proteome of the diseased heart, which can be an aspect of the treatment strategy. The ECM particles may be treated to improve crosslinking, stability, injectability, or compatibility with a patient, which can enhance their effectiveness in altering the proteome of the diseased heart.

In the context of altering the proteome of a diseased heart, the process may involve the restoration of a normal ratio of levels of different collagens within the heart. This restoration may be achieved through the interaction of extra-cellular matrix (ECM) particles with the heart tissue. The ECM particles, which may be produced from decellularized 3D microtissues, can be designed to restore the balance of collagen types I, III, and IV. This balance may promote proper cardiac function and potentially prevent fibrosis. The restoration process may be facilitated by the ECM particles' ability to interact with the heart's existing collagen structures, thereby adjusting the levels to a more normal state. The ECM particles may be engineered to retain bioactive components and structural properties that are conducive to this restoration. The process may also involve the modulation of cellular activities that influence collagen production and degradation, ensuring that the heart's collagen levels are maintained within a range that supports optimal cardiac function. This approach may offer a method to address the imbalance of collagen levels that is often associated with cardiac diseases, potentially leading to improved cardiac health and function.

The immune response in the heart may be altered by contacting the diseased heart with extra-cellular matrix (ECM) particles produced from decellularized 3D microtissues. This alteration may involve the modulation of the activity of immune cells such as T cells, B cells, and macrophages. The modulation of these immune cells may lead to a reduction in inflammation and may promote tissue repair. The ECM particles may interact with the immune cells, potentially influencing their behavior and activity within the cardiac tissue. The alteration of the immune response may be facilitated by the bioactive components retained within the ECM particles, which may interact with the cellular environment of the heart. The process may involve complex interactions between the ECM particles and the immune cells, potentially leading to changes in the signaling pathways that regulate inflammation and tissue repair. The modulation of the immune response may be a step in the overall method of altering the proteome of a diseased heart, as it may contribute to the restoration of normal cardiac function and the prevention of further damage to the heart tissue. The potential for altering the immune response may be an aspect of the method, as it may provide a means to address the underlying causes of inflammation and promote healing in the diseased heart.

In the context of altering the proteome of a diseased heart, the process may involve the alteration of metabolism within the heart. This alteration can be achieved by engaging with the mitochondrial function, which may enhance the energy supply to the cardiac tissue. The metabolism and mitochondrial function may be involved in this process. The alteration of metabolism may be facilitated by the contacting of the diseased heart with extra-cellular matrix (ECM) particles produced from decellularized 3D microtissues. These ECM particles may be derived from human mesenchymal stem cells, which may retain the bioactive components and structural properties of the native tissue. The alteration of metabolism may potentially improve mitochondrial function, increase ATP production, and reduce oxidative stress, thereby enhancing the energy supply to the cardiac tissue. This process may be part of a broader method to alter the proteome of a diseased heart, which may also involve restoring the normal ratio of levels of different collagens, altering the immune response, and mitigating the effects of TGF-Beta, a growth factor associated with fibrosis. The method may not be limited to these actions and may include other potential actions that contribute to the treatment of a diseased heart.

In the context of the system, the effects of TGF-Beta in the heart may be mitigated through the application of extra-cellular matrix (ECM) particles. These particles, which may be produced from decellularized 3D microtissues, can be contacted with the diseased heart. This contacting may lead to a reduction in the activation of fibroblasts and the deposition of excessive extracellular matrix components. The mitigation of TGF-Beta effects may be in addressing fibrosis, a condition associated with the growth factor TGF-Beta. The ECM particles may retain bioactive components and structural properties that are for this process. The method may involve the use of human mesenchymal stem cells as a source for the 3D microtissues, which may be cultured under specific conditions to promote the formation of tissue-specific extracellular matrix. This approach may offer a potential pathway to alter the proteome of a diseased heart, thereby contributing to the reduction of fibrosis and the improvement of cardiac function. The actions described may be aligned with the method, suggesting a comprehensive strategy to address heart disease through the modulation of TGF-Beta effects.

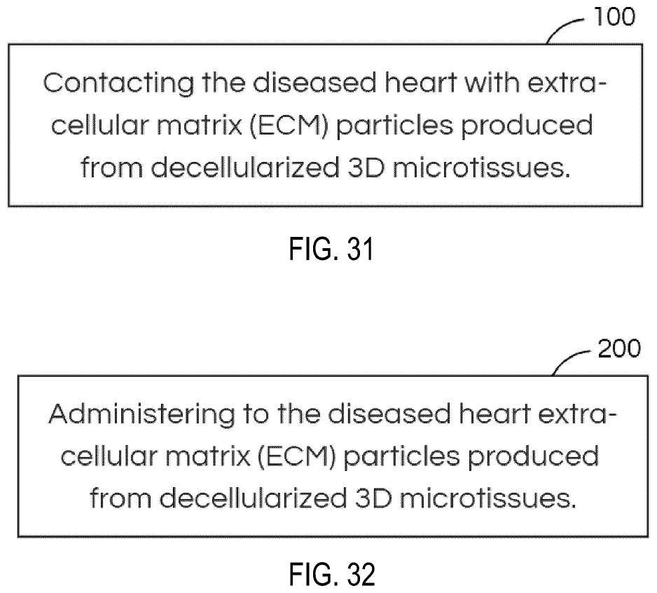

FIG. 31 is a flowchart illustrating a method in step 100 for altering the proteome of a diseased heart by contacting it with extra-cellular matrix (ECM) particles produced from decellularized 3D microtissues, according to an embodiment. At step 100, the diseased heart may be contacted with ECM particles, which are produced from decellularized 3D microtissues. The ECM particles may be derived from human mesenchymal stem cells, which may be cultured under conditions that promote the formation of tissue-specific extracellular matrix. This contacting may alter the proteome of the diseased heart. The contacting may restore a normal ratio of levels of different collagens in the heart, which may include collagen types I, III, and IV. This restoration may promote proper cardiac function and may prevent fibrosis. Additionally, the contacting may alter an immune response in the heart by modulating the activity of macrophages, T cells, and B cells, which may lead to a reduction in inflammation and may promote tissue repair. Furthermore, the contacting may alter metabolism in the heart by improving mitochondrial function, increasing ATP production, and reducing oxidative stress, thereby potentially enhancing the energy supply to the cardiac tissue. The contacting may also mitigate the effects of TGF-Beta in the heart, a growth factor associated with fibrosis, which may reduce the activation of fibroblasts and the deposition of excessive extracellular matrix components. The ECM particles may retain the bioactive components and structural properties of the native tissue, which may be for the described alterations in the proteome of the diseased heart.

FIG. 32 is a flowchart illustrating a method in step 200 for administering extra-cellular matrix (ECM) particles to a diseased heart, according to an embodiment. At step 200, the diseased heart may be administered with ECM particles produced from decellularized 3D microtissues. The administration of these ECM particles may potentially restore a normal ratio of levels of different collagens in the heart, which may promote proper cardiac function and prevent fibrosis. Additionally, the administration may alter the immune response in the heart by modulating the activity of macrophages, T cells, and B cells, which may lead to a reduction in inflammation and promote tissue repair. Furthermore, the administration may alter metabolism in the heart by improving mitochondrial function, increasing ATP production, and reducing oxidative stress, thereby potentially enhancing the energy supply to the cardiac tissue. The effects of TGF-Beta, a growth factor associated with fibrosis, may also be mitigated through this administration, which may reduce the activation of fibroblasts and the deposition of excessive extracellular matrix components. The 3D microtissues from which the ECM particles are derived may originate from human mesenchymal stem cells, which may be cultured under conditions that promote the formation of tissue-specific extracellular matrix, ensuring that the resulting ECM particles retain the bioactive components and structural properties of the native tissue.

The methods described in this disclosure have the potential to save human lives by addressing the underlying causes of heart disease, which is a leading cause of mortality worldwide. By altering the proteome of a diseased heart, these methods can restore normal cardiac function, reduce inflammation, and prevent fibrosis, all of which are critical factors in the progression of heart disease. The restoration of normal collagen levels in the heart can prevent the stiffening of cardiac tissue, which is a common consequence of fibrosis and can lead to heart failure. By maintaining the flexibility and elasticity of the heart, these methods can improve the heart's ability to pump blood effectively, reducing the risk of heart failure and other complications.

The modulation of the immune response in the heart can also play a significant role in saving lives. Inflammation is a key driver of heart disease, and by reducing inflammation and promoting tissue repair, these methods can prevent further damage to the heart and promote healing. This can lead to improved cardiac function and a reduced risk of heart attacks and other life-threatening events.

The enhancement of metabolism in the heart is another critical aspect of these methods. By improving mitochondrial function and increasing ATP production, these methods can ensure that the heart has an adequate energy supply to function properly. This can prevent the energy deficits that often occur in heart disease and lead to heart failure. By reducing oxidative stress, these methods can also protect the heart from damage caused by free radicals, further preserving cardiac function.

The mitigation of TGF-Beta effects in the heart can also contribute to saving lives by preventing the excessive deposition of extracellular matrix components, which can lead to fibrosis and heart failure. By reducing the activation of fibroblasts, these methods can prevent the progression of fibrosis and maintain the structural integrity of the heart.

Overall, the methods described in this disclosure offer a comprehensive approach to treating heart disease by addressing multiple underlying causes and promoting the restoration of normal cardiac function. By doing so, these methods have the potential to save lives and improve the quality of life for individuals with heart disease.

Additional inter-combinable details: when contemplating the details of the Invention disclosed herein, in a discussion, study or a reading of the details, features, embodiments, aspects, and/or examples of the technology disclosed herein, any of the features, embodiments, aspects, and/or examples can be inter-combined, inter-claimed (or inter-discussed) with the example details listed below:

Detail 1: A method of altering the proteome of a diseased heart, comprising: contacting the diseased heart with extra-cellular matrix (ECM) particles produced from decellularized 3D microtissues, wherein the contacting alters the proteome of the diseased heart and beneficially changes expression of one or more genes selected from the group consisting of NPPA, NPPB, MYH6, MYH7, ACTC1, TNNT2, TNNC1, TNNI3, TPM1, MYL2, MYL3, MYL7, MYBPC3, TTN, ACTN2, ANKRD1, CSRP3, LDB3, TCAP, VCL, DES, LMNA, RBM20, PLN, SLC8A1, SLC9A1, HRC, CASQ2, PRKAG2, and LAMP2.

Detail 2: The method of detail 1, wherein the contacting restores a normal ratio of levels of different collagens in the heart and beneficially changes expression of one or more genes selected from the group consisting of COL1A1, COL1A2, COL3A1, COL4A1, COL4A2, COL4A3, COL4A4, COL4A5, COL4A6, COL5A1, COL5A2, COL6A1, COL6A2, COL6A3, COL6A5, COL6A6, COL8A1, COL8A2, COL14A1, COL15A1, COL18A1, and COL21A1.

Detail 3: The method of detail 1, wherein the contacting alters an immune response in the heart and beneficially changes expression of one or more genes selected from the group consisting of IL1B, IL6, TNF, IFNG, IL2, IL4, IL10, IL13, IL17A, TGFB1, CCL2, CCL3, CCL4, CCL5, CCL11, CXCL8, CXCL10, CX3CL1, ICAM1, VCAM1, SELE, SELP, CD14, TLR2, TLR4, MRC1, and CD163.

Detail 4: The method of detail 1, wherein the contacting alters metabolism in the heart and beneficially changes expression of one or more genes selected from the group consisting of PPARA, PPARG, PPARGC1A, PPARGC1B, ESRRA, ESRRB, ESRRG, NRF1, TFAM, CPT1A, CPT1B, ACADM, ACADVL, HADHA, HADHB, IDH2, ACO2, SDHA, SDHB, SDHC, SDHD, ATP5F1A, ATP5F1B, ATP5F1C, ATP5F1D, ATP5F1E, ATP5MC1, ATP5MC2, ATP5MC3, ATP5PB, ATP5PD, ATP5PF, ATP5PO, and ATP5ME.

Detail 5: The method of detail 1, wherein the contacting mitigates effects of TGF-Beta in the heart, wherein TGF-Beta is a growth factor associated with fibrosis, and beneficially changes expression of one or more genes selected from the group consisting of TGFB1, TGFB2, TGFB3, TGFBR1, TGFBR2, TGFBR3, SMAD2, SMAD3, SMAD4, SMAD6, SMAD7, SERPINE1, THBS1, THBS2, THBS3, THBS4, LTBP1, LTBP2, LTBP3, LTBP4, FN1, CTGF, and PDGFB.

Detail 6: The method of detail 1, wherein the 3D microtissues are derived from human mesenchymal stem cells and the contacting beneficially changes expression of one or more genes selected from the group consisting of NT5E, ENG, THY1, ALCAM, VCAM1, ITGAV, ITGB1, VEGFA, VEGFB, VEGFC, PGF, PDGFRA, PDGFRB, FGF2, EGF, IGF1, BDNF, NGF, NTF3, NTF4, IL1B, IL6, IL10, TNF, IFNG, CCL2, CCL5, and CXCL12.

Detail 7: A method of treating a diseased heart, comprising: administering to the diseased heart extra-cellular matrix (ECM) particles produced from decellularized 3D microtissues, wherein the administering treats the diseased heart by altering the proteome of the diseased heart and beneficially changing expression of one or more genes selected from the group consisting of NPPA, NPPB, MYH6, MYH7, ACTC1, TNNT2, TNNC1, TNNI3, TPM1, MYL2, MYL3, MYL7, MYBPC3, TTN, ACTN2, ANKRD1, CSRP3, LDB3, TCAP, VCL, DES, LMNA, RBM20, PLN, SLC8A1, SLC9A1, HRC, CASQ2, PRKAG2, and LAMP2.

Detail 8: The method of detail 7, wherein the administering restores a normal ratio of levels of different collagens in the heart and beneficially changes expression of one or more genes selected from the group consisting of COL1A1, COL1A2, COL3A1, COL4A1, COL4A2, COL4A3, COL4A4, COL4A5, COL4A6, COL5A1, COL5A2, COL6A1, COL6A2, COL6A3, COL6A5, COL6A6, COL8A1, COL8A2, COL14A1, COL15A1, COL18A1, and COL21A1.

Detail 9: The method of detail 7, wherein the administering alters an immune response in the heart and beneficially changes expression of one or more genes selected from the group consisting of IL1B, IL6, TNF, IFNG, IL2, IL4, IL10, IL13, IL17A, TGFB1, CCL2, CCL3, CCL4, CCL5, CCL11, CXCL8, CXCL10, CX3CL1, ICAM1, VCAM1, SELE, SELP, CD14, TLR2, TLR4, MRC1, and CD163.

Detail 10: The method of detail 7, wherein the administering alters metabolism in the heart and beneficially changes expression of one or more genes selected from the group consisting of PPARA, PPARG, PPARGC1A, PPARGC1B, ESRRA, ESRRB, ESRRG, NRF1, TFAM, CPT1A, CPT1B, ACADM, ACADVL, HADHA, HADHB, IDH2, ACO2, SDHA, SDHB, SDHC, SDHD, ATP5F1A, ATP5F1B, ATP5F1C, ATP5F1D, ATP5F1E, ATP5MC1, ATP5MC2, ATP5MC3, ATP5PB, ATP5PD, ATP5PF, ATP5PO, and ATP5ME.

Detail 11: The method of detail 7, wherein the administering mitigates effects of TGF-Beta in the heart, wherein TGF-Beta is a growth factor associated with fibrosis, and beneficially changes expression of one or more genes selected from the group consisting of TGFB1, TGFB2, TGFB3, TGFBR1, TGFBR2, TGFBR3, SMAD2, SMAD3, SMAD4, SMAD6, SMAD7, SERPINE1, THBS1, THBS2, THBS3, THBS4, LTBP1, LTBP2, LTBP3, LTBP4, FN1, CTGF, and PDGFB.

Detail 12: The method of detail 7, wherein the 3D microtissues are derived from human mesenchymal stem cells and the administering beneficially changes expression of one or more genes selected from the group consisting of NT5E, ENG, THY1, ALCAM, VCAM1, ITGAV, ITGB1, VEGFA, VEGFB, VEGFC, PGF, PDGFRA, PDGFRB, FGF2, EGF, IGF1, BDNF, NGF, NTF3, NTF4, IL1B, IL6, IL10, TNF, IFNG, CCL2, CCL5, and CXCL12.

Detail 13: Extra-cellular matrix (ECM) particles for altering the proteome of a diseased heart, wherein the ECM particles are produced from decellularized 3D microtissues and treated to improve one or more of crosslinking, stability, injectability, or compatibility with a patient, and wherein the ECM particles beneficially change expression of one or more genes selected from the group consisting of NPPA, NPPB, MYH6, MYH7, ACTC1, TNNT2, TNNC1, TNNI3, TPM1, MYL2, MYL3, MYL7, MYBPC3, TTN, ACTN2, ANKRD1, CSRP3, LDB3, TCAP, VCL, DES, LMNA, RBM20, PLN, SLC8A1, SLC9A1, HRC, CASQ2, PRKAG2, and LAMP2.

Detail 14: The ECM particles of detail 13, wherein the ECM particles, when contacted with the diseased heart, restore a normal ratio of levels of different collagens in the heart and beneficially change expression of one or more genes selected from the group consisting of COL1A1, COL1A2, COL3A1, COL4A1, COL4A2, COL4A3, COL4A4, COL4A5, COL4A6, COL5A1, COL5A2, COL6A1, COL6A2, COL6A3, COL6A5, COL6A6, COL8A1, COL8A2, COL14A1, COL15A1, COL18A1, and COL21A1.

Detail 15: The ECM particles of detail 13, wherein the ECM particles, when contacted with the diseased heart, alter an immune response in the heart and beneficially change expression of one or more genes selected from the group consisting of IL1B, IL6, TNF, IFNG, IL2, IL4, IL10, IL13, IL17A, TGFB1, CCL2, CCL3, CCL4, CCL5, CCL11, CXCL8, CXCL10, CX3CL1, ICAM1, VCAM1, SELE, SELP, CD14, TLR2, TLR4, MRC1, and CD163.

Detail 16: The ECM particles of detail 13, wherein the ECM particles, when contacted with the diseased heart, alter metabolism in the heart and beneficially change expression of one or more genes selected from the group consisting of PPARA, PPARG, PPARGC1A, PPARGC1B, ESRRA, ESRRB, ESRRG, NRF1, TFAM, CPT1A, CPT1B, ACADM, ACADVL, HADHA, HADHB, IDH2, ACO2, SDHA, SDHB, SDHC, SDHD, ATP5F1A, ATP5F1B, ATP5F1C, ATP5F1D, ATP5F1E, ATP5MC1, ATP5MC2, ATP5MC3, ATP5PB, ATP5PD, ATP5PF, ATP5PO, and ATP5ME.

Detail 17: The ECM particles of detail 13, wherein the ECM particles, when contacted with the diseased heart, mitigate effects of TGF-Beta in the heart, wherein TGF-Beta is a growth factor associated with fibrosis, and beneficially change expression of one or more genes selected from the group consisting of TGFB1, TGFB2, TGFB3, TGFBR1, TGFBR2, TGFBR3, SMAD2, SMAD3, SMAD4, SMAD6, SMAD7, SERPINE1, THBS1, THBS2, THBS3, THBS4, LTBP1, LTBP2, LTBP3, LTBP4, FN1, CTGF, and PDGFB.

Detail 18: The ECM particles of detail 13, wherein the 3D microtissues are derived from human mesenchymal stem cells and the ECM particles beneficially change expression of one or more genes selected from the group consisting of NT5E, ENG, THY1, ALCAM, VCAM1, ITGAV, ITGB1, ITGB3, ITGB5, ANPEP, ENPEP, MCAM, NCAM1, CD44, HGF, BMP2, WNT5A, TGFB1, TGFB3, VEGFA, VEGFB, VEGFC, PGF, PDGFRA, PDGFRB, FGF2, EGF, IGF1, BDNF, NGF, NTF3, NTF4, IL1B, IL6, IL10, TNF, IFNG, CCL2, CCL5, and CXCL12.

Detail 19: A composition for treating a diseased heart, comprising: extra-cellular matrix (ECM) particles produced from decellularized 3D microtissues and treated to improve one or more of crosslinking, stability, injectability, or compatibility with a patient; wherein the composition, when administered to the diseased heart, treats the diseased heart by altering the proteome of the diseased heart and beneficially changing expression of one or more genes selected from the group consisting of NPPA, NPPB, MYH6, MYH7, ACTC1, TNNT2, TNNC1, TNNI3, TPM1, MYL2, MYL3, MYL7, MYBPC3, TTN, ACTN2, ANKRD1, CSRP3, LDB3, TCAP, VCL, DES, LMNA, RBM20, PLN, SLC8A1, SLC9A1, HRC, CASQ2, PRKAG2, and LAMP2.

Detail 20: The composition of detail 19, wherein the 3D microtissues are derived from human mesenchymal stem cells and the composition beneficially changes expression of one or more genes selected from the group consisting of NT5E, ENG, THY1, ALCAM, VCAM1, ITGAV, ITGB1, ITGB3, ITGB5, ANPEP, ENPEP, MCAM, NCAM1, CD44, HGF, BMP2, WNT5A, TGFB1, TGFB3, VEGFA, VEGFB, VEGFC, PGF, PDGFRA, PDGFRB, FGF2, EGF, IGF1, and BDNF.

Detail 21: A method for altering the proteome of a diseased heart, comprising: contacting the diseased heart with extra-cellular matrix (ECM) particles produced from decellularized 3D microtissues of human mesenchymal stem cells, wherein the contacting modifies the proteome, adjusts the ratio of collagen levels, modulates the immune response, changes metabolism, or reduces the effects of fibrosis-associated factors in the diseased heart.

Detail 22: The method of detail 21, wherein the extra-cellular matrix (ECM) particles are derived from decellularized 3D tissue structures originating from mesenchymal stem cells, and wherein the mesenchymal stem cells are isolated from a tissue source selected from the group consisting of bone marrow, adipose tissue, umbilical cord blood, umbilical cord tissue, Wharton's jelly, placenta, dental pulp, peripheral blood, and skin, and wherein the mesenchymal stem cells are expanded in vitro under conditions that maintain their multipotency and ability to differentiate into various cell types, and wherein the 3D tissue structures are formed by seeding the mesenchymal stem cells onto a biocompatible scaffold material and culturing the seeded scaffold under conditions that promote the formation of a 3D tissue structure, and wherein the decellularization process involves treating the 3D tissue structure with one or more decellularization agents selected from the group consisting of detergents, enzymes, and hypertonic solutions, and wherein the decellularization process removes cellular components while preserving the extracellular matrix components and structure.

Detail 23: The method of detail 21, wherein the fibrosis-associated factors comprise TGF-Beta, and wherein TGF-Beta is a multifunctional cytokine that plays a key role in the development of fibrosis by promoting the activation and proliferation of fibroblasts, stimulating the synthesis and deposition of extracellular matrix components such as collagen, and inhibiting the degradation of extracellular matrix components, and wherein the reduction of TGF-Beta effects by the ECM particles helps to attenuate the fibrotic process in the diseased heart.

Detail 24: The method of detail 21, wherein the contacting restores the normal ratio of the levels of different collagens in the heart, and wherein the different collagens include collagen type I, collagen type III, and collagen type V, and wherein the normal ratio of collagen types I, III, and V in the heart is disrupted in various cardiac pathologies, leading to abnormal cardiac function and structure, and wherein the restoration of the normal collagen ratio by the ECM particles helps to improve cardiac function and structure in the diseased heart.

Detail 25: The method of detail 21, wherein the contacting alters the immune response in the heart, and wherein the altered immune response includes changes in the levels and/or activities of various immune cells and factors, such as macrophages, T cells, B cells, neutrophils, cytokines, and chemokines, and wherein the modulation of the immune response by the ECM particles helps to reduce inflammation and promote tissue repair in the diseased heart.

Detail 26: The method of detail 21, wherein the contacting alters metabolism in the heart, and wherein the altered metabolism includes changes in the levels and/or activities of various metabolic enzymes, substrates, and products involved in energy production, such as glucose, fatty acids, pyruvate, lactate, and ATP, and wherein the modulation of cardiac metabolism by the ECM particles helps to improve energy efficiency and reduce oxidative stress in the diseased heart.

Detail 27: The method of detail 21, wherein the decellularized 3D microtissues are produced by decellularizing 3D microtissues of human mesenchymal stem cells, and wherein the 3D microtissues are formed by seeding human mesenchymal stem cells onto a microwell culture platform and culturing the seeded cells under conditions that promote the formation of multicellular aggregates with a diameter of approximately 100-500 μm, and wherein the decellularization process involves treating the 3D microtissues with a combination of freeze-thaw cycles, detergents, and enzymes to remove the cellular components while preserving the extracellular matrix components and structure.

Detail 28: The method of detail 27, wherein the 3D microtissues are produced by culturing human mesenchymal stem cells in a 3D environment, and wherein the 3D environment is created using a hydrogel matrix, a porous polymer scaffold, a microfluidic device, or a bioreactor system, and wherein the 3D environment provides mechanical and biochemical cues that mimic the native tissue microenvironment and promote the differentiation and functional maturation of the mesenchymal stem cells.

Detail 29: The method of detail 21, wherein the ECM particles are in the form of a powder, suspension, or solution, and wherein the powder is obtained by lyophilizing the decellularized 3D microtissues and grinding the lyophilized tissue into a fine powder, and wherein the suspension is obtained by resuspending the powder in a suitable aqueous buffer or culture medium, and wherein the solution is obtained by dissolving the powder in a suitable solvent or enzymatically digesting the decellularized 3D microtissues.

Detail 30: The method of detail 21, wherein the contacting is performed by injecting the ECM particles into the diseased heart, and wherein the injection is performed using a needle, catheter, or other suitable delivery device, and wherein the injection site is selected based on the location and extent of the cardiac injury or pathology, and wherein the injection may be performed as a single bolus or as multiple injections over time.

Detail 31: The method of detail 21, wherein the contacting is performed by applying the ECM particles to the surface of the diseased heart, and wherein the application is performed by spraying, painting, or otherwise coating the surface of the heart with a solution or suspension containing the ECM particles, and wherein the application may be performed during open heart surgery or using a minimally invasive surgical approach.

Detail 32: The method of detail 21, wherein the diseased heart is affected by a condition selected from the group consisting of myocardial infarction, heart failure, and cardiomyopathy, and wherein myocardial infarction is characterized by the death of cardiac tissue due to a blockage in the coronary arteries that supply blood to the heart, and wherein heart failure is characterized by the inability of the heart to pump enough blood to meet the body's needs, and wherein cardiomyopathy is characterized by a weakening or change in the structure of the heart muscle that affects its ability to pump blood effectively.

Detail 33: The method of detail 21, wherein the contacting is performed in vivo, and wherein the in vivo contacting involves administering the ECM particles to a living subject, either locally at the site of cardiac injury or systemically via the circulation, and wherein the in vivo contacting allows the ECM particles to interact with the native cardiac tissue and exert their therapeutic effects in the context of the living organism.

Detail 34: The method of detail 21, wherein the contacting is performed ex vivo, and wherein the ex vivo contacting involves exposing isolated cardiac cells, tissues, or organs to the ECM particles in a cell culture dish, bioreactor, or other suitable in vitro system, and wherein the ex vivo contacting allows for more precise control over the dosage, timing, and localization of the ECM particles and enables the study of their effects on cardiac cells and tissues in a simplified and standardized environment.

Detail 35: The method of detail 21, wherein the ECM particles are autologous to the subject with the diseased heart, and wherein the autologous ECM particles are derived from decellularized 3D microtissues that are produced using mesenchymal stem cells obtained from the same subject, and wherein the use of autologous ECM particles reduces the risk of immune rejection and enhances the biocompatibility and integration of the particles with the host cardiac tissue.

Detail 36: The method of detail 21, wherein the ECM particles are allogeneic to the subject with the diseased heart, and wherein the allogeneic ECM particles are derived from decellularized 3D microtissues that are produced using mesenchymal stem cells obtained from a different human donor, and wherein the use of allogeneic ECM particles allows for the large-scale production and off-the-shelf availability of the therapeutic product, and wherein the decellularization process removes the immunogenic cellular components and reduces the risk of immune rejection.

Detail 37: The method of detail 21, wherein the human mesenchymal stem cells are derived from a tissue selected from the group consisting of bone marrow, adipose tissue, and umbilical cord, and wherein bone marrow-derived mesenchymal stem cells are obtained by aspiration of bone marrow from the iliac crest or other suitable site and isolation of the mononuclear cell fraction, and wherein adipose tissue-derived mesenchymal stem cells are obtained by enzymatic digestion of lipoaspirate or other adipose tissue samples and isolation of the stromal vascular fraction, and wherein umbilical cord-derived mesenchymal stem cells are obtained by enzymatic digestion of umbilical cord tissue, such as Wharton's jelly or umbilical cord blood, and isolation of the mononuclear cell fraction.

Detail 38: The method of detail 21, wherein the ECM particles are lyophilized, and wherein the lyophilization process involves freezing the decellularized 3D microtissues and subliming the water content under vacuum to obtain a dry powder, and wherein the lyophilization process preserves the structural and functional integrity of the ECM components and enhances the stability and shelf-life of the therapeutic product.

Detail 39: The method of detail 21, wherein the ECM particles are crosslinked, and wherein the crosslinking process involves treating the decellularized 3D microtissues with a crosslinking agent, such as glutaraldehyde, genipin, or riboflavin, and wherein the crosslinking process stabilizes the ECM structure, improves its mechanical properties, and reduces its susceptibility to enzymatic degradation, and wherein the degree of crosslinking can be controlled by adjusting the concentration and duration of exposure to the crosslinking agent.

Detail 40: A composition for altering the proteome of a diseased heart, comprising: extra-cellular matrix (ECM) particles produced from decellularized 3D microtissues of human mesenchymal stem cells, wherein the ECM particles are capable of modifying the proteome, adjusting the ratio of collagen levels, modulating the immune response, changing metabolism, or reducing the effects of fibrosis-associated factors when contacted with the diseased heart, and wherein the composition is formulated as a powder, suspension, solution, gel, or other suitable dosage form for local or systemic administration to the diseased heart, and wherein the composition may further comprise one or more pharmaceutically acceptable carriers, excipients, diluents, or other inactive ingredients to enhance its stability, deliverability, or therapeutic efficacy.

Detail 41: A method of altering the proteome of a diseased heart in a patient in need thereof, the method comprising: contacting the diseased heart with a therapeutically effective amount of extra-cellular matrix (ECM) particles produced from decellularized three-dimensional (3D) microtissues, wherein the ECM particles have an average diameter of about 10 nm to about 1000 μm, wherein the 3D microtissues are derived from human mesenchymal stem cells cultured under conditions that promote the production of ECM, wherein the decellularized 3D microtissues are treated to remove cellular components while preserving ECM components, wherein the ECM particles are sterilized and lyophilized to improve stability and injectability, wherein the contacting comprises injecting the ECM particles into one or more regions of the diseased heart, wherein, upon contact with the diseased heart, the ECM particles alter the proteome of the diseased heart by modulating the expression or activity of one or more proteins selected from the group consisting of collagens, elastin, fibronectin, laminin, matricellular proteins, proteoglycans, and glycoproteins, wherein the alteration of the proteome improves the structure and/or function of the diseased heart.

Detail 42: The method of detail 41, wherein the contacting restores a normal ratio of levels of different collagens in the heart, the different collagens selected from the group consisting of collagen I, collagen III, collagen IV, collagen V, collagen VI, collagen VII, collagen VIII, collagen IX, collagen X, collagen XI, collagen XII, collagen XIII, collagen XIV, collagen XV, collagen XVI, collagen XVII, collagen XVIII, collagen XIX, collagen XX, collagen XXI, collagen XXII, collagen XXIII, collagen XXIV, collagen XXV, collagen XXVI, collagen XXVII, and collagen XXVIII.

Detail 43: The method of detail 41, wherein the contacting alters an immune response in the heart by modulating the expression or activity of one or more cytokines, chemokines, growth factors, or immune cell markers selected from the group consisting of TNF-α, IL-1β, IL-6, IL-10, IL-8, MCP-1, MIP-1a, MIP-1B, CXCL1, CXCL2, CXCL5, TGF-β1, TGF-β2, TGF-β3, IFN-γ, CD68, CD45, CD3, CD4, CD8, and CD20.

Detail 44: The method of detail 41, wherein the contacting alters metabolism in the heart by modulating the expression or activity of one or more metabolic enzymes or transporters selected from the group consisting of pyruvate dehydrogenase, citrate synthase, isocitrate dehydrogenase, α-ketoglutarate dehydrogenase, succinate dehydrogenase, malate dehydrogenase, ATP synthase, GLUT1, GLUT4, CPT1, and CPT2.

Detail 45: The method of detail 41, wherein the contacting mitigates effects of TGF-Beta in the heart by reducing the expression or activity of TGF-Beta isoforms, receptors, or downstream signaling molecules, wherein TGF-Beta is a growth factor associated with fibrosis.

Detail 46: The method of detail 41, wherein the human mesenchymal stem cells are isolated from one or more tissues selected from the group consisting of bone marrow, adipose tissue, umbilical cord blood, umbilical cord tissue, peripheral blood, dental pulp, and synovial fluid.

Detail 47: A method of treating a diseased heart in a patient in need thereof, the method comprising: administering to the diseased heart a therapeutically effective amount of extra-cellular matrix (ECM) particles produced from decellularized three-dimensional (3D) microtissues, wherein the ECM particles have an average diameter of about 10 nm to about 1000 μm, wherein the 3D microtissues are derived from human mesenchymal stem cells cultured under conditions that promote the production of ECM, wherein the decellularized 3D microtissues are treated to remove cellular components while preserving ECM components, wherein the ECM particles are sterilized and lyophilized to improve stability and injectability, wherein the administering comprises injecting the ECM particles into one or more regions of the diseased heart, wherein, upon administration to the diseased heart, the ECM particles treat the diseased heart by altering the proteome of the diseased heart and improving the structure and/or function of the diseased heart.

Detail 48: The method of detail 47, wherein the administering restores a normal ratio of levels of different collagens in the heart, the different collagens selected from the group consisting of collagen I, collagen III, collagen IV, collagen V, collagen VI, collagen VII, collagen VIII, collagen IX, collagen X, collagen XI, collagen XII, collagen XIII, collagen XIV, collagen XV, collagen XVI, collagen XVII, collagen XVIII, collagen XIX, collagen XX, collagen XXI, collagen XXII, collagen XXIII, collagen XXIV, collagen XXV, collagen XXVI, collagen XXVII, and collagen XXVIII.

Detail 49: The method of detail 47, wherein the administering alters an immune response in the heart by modulating the expression or activity of one or more cytokines, chemokines, growth factors, or immune cell markers selected from the group consisting of TNF-α, IL-1β, IL-6, IL-10, IL-8, MCP-1, MIP-1α, MIP-1β, CXCL1, CXCL2, CXCL5, TGF-31, TGF-β2, TGF-β3, IFN-γ, CD68, CD45, CD3, CD4, CD8, and CD20.

Detail 50: The method of detail 47, wherein the administering alters metabolism in the heart by modulating the expression or activity of one or more metabolic enzymes or transporters selected from the group consisting of pyruvate dehydrogenase, citrate synthase, isocitrate dehydrogenase, α-ketoglutarate dehydrogenase, succinate dehydrogenase, malate dehydrogenase, ATP synthase, GLUT1, GLUT4, CPT1, and CPT2.

Detail 51: The method of detail 47, wherein the administering mitigates effects of TGF-Beta in the heart by reducing the expression or activity of TGF-Beta isoforms, receptors, or downstream signaling molecules, wherein TGF-Beta is a growth factor associated with fibrosis.

Detail 52: The method of detail 47, wherein the human mesenchymal stem cells are isolated from one or more tissues selected from the group consisting of bone marrow, adipose tissue, umbilical cord blood, umbilical cord tissue, peripheral blood, dental pulp, and synovial fluid.

Detail 53: Extra-cellular matrix (ECM) particles for altering the proteome of a diseased heart, wherein the ECM particles are produced from decellularized three-dimensional (3D) microtissues and treated to improve one or more of crosslinking, stability, injectability, or compatibility with a patient, wherein the ECM particles have an average diameter of about 10 nm to about 1000 μm, wherein the 3D microtissues are derived from human mesenchymal stem cells cultured under conditions that promote the production of ECM, wherein the decellularized 3D microtissues are treated to remove cellular components while preserving ECM components, wherein the ECM particles are sterilized and lyophilized to improve stability and injectability, wherein, upon contact with the diseased heart, the ECM particles alter the proteome of the diseased heart by modulating the expression or activity of one or more proteins selected from the group consisting of collagens, elastin, fibronectin, laminin, matricellular proteins, proteoglycans, and glycoproteins.

Detail 54: The ECM particles of detail 53, wherein the ECM particles, when contacted with the diseased heart, restore a normal ratio of levels of different collagens in the heart, the different collagens selected from the group consisting of collagen I, collagen III, collagen IV, collagen V, collagen VI, collagen VII, collagen VIII, collagen IX, collagen X, collagen XI, collagen XII, collagen XIII, collagen XIV, collagen XV, collagen XVI, collagen XVII, collagen XVIII, collagen XIX, collagen XX, collagen XXI, collagen XXII, collagen XXIII, collagen XXIV, collagen XXV, collagen XXVI, collagen XXVII, and collagen XXVIII.

Detail 55: The ECM particles of detail 53, wherein the ECM particles, when contacted with the diseased heart, alter an immune response in the heart by modulating the expression or activity of one or more cytokines, chemokines, growth factors, or immune cell markers selected from the group consisting of TNF-α, IL-1β, IL-6, IL-10, IL-8, MCP-1, MIP-1α, MIP-1β, CXCL1, CXCL2, CXCL5, TGF-β1, TGF-β2, TGF-β3, IFN-γ, CD68, CD45, CD3, CD4, CD8, and CD20.

Detail 56: The ECM particles of detail 53, wherein the ECM particles, when contacted with the diseased heart, alter metabolism in the heart by modulating the expression or activity of one or more metabolic enzymes or transporters selected from the group consisting of pyruvate dehydrogenase, citrate synthase, isocitrate dehydrogenase, α-ketoglutarate dehydrogenase, succinate dehydrogenase, malate dehydrogenase, ATP synthase, GLUT1, GLUT4, CPT1, and CPT2.

Detail 57: The ECM particles of detail 53, wherein the ECM particles, when contacted with the diseased heart, mitigate effects of TGF-Beta in the heart by reducing the expression or activity of TGF-Beta isoforms, receptors, or downstream signaling molecules, wherein TGF-Beta is a growth factor associated with fibrosis.

Detail 58: The ECM particles of detail 53, wherein the human mesenchymal stem cells are isolated from one or more tissues selected from the group consisting of bone marrow, adipose tissue, umbilical cord blood, umbilical cord tissue, peripheral blood, dental pulp, and synovial fluid.

Detail 59: A composition for treating a diseased heart, comprising: extra-cellular matrix (ECM) particles produced from decellularized three-dimensional (3D) microtissues and treated to improve one or more of crosslinking, stability, injectability, or compatibility with a patient, wherein the ECM particles have an average diameter of about 10 nm to about 1000 μm, wherein the 3D microtissues are derived from human mesenchymal stem cells cultured under conditions that promote the production of ECM, wherein the decellularized 3D microtissues are treated to remove cellular components while preserving ECM components, wherein the ECM particles are sterilized and lyophilized to improve stability and injectability, wherein the composition, when administered to the diseased heart, treats the diseased heart by altering the proteome of the diseased heart and improving the structure and/or function of the diseased heart.

Detail 60: The composition of detail 59, wherein the human mesenchymal stem cells are isolated from one or more tissues selected from the group consisting of bone marrow, adipose tissue, umbilical cord blood, umbilical cord tissue, peripheral blood, dental pulp, and synovial fluid.

Detail 61: A method of altering the proteome of a diseased heart, comprising: contacting the diseased heart with extra-cellular matrix (ECM) particles produced from decellularized 3D microtissues, wherein the contacting alters the proteome of the diseased heart.

Detail 62: The method of detail 61, wherein the contacting restores a normal ratio of levels of different collagens in the heart.

Detail 63: The method of detail 61, wherein the contacting alters an immune response in the heart.

Detail 64: The method of detail 61, wherein the contacting alters metabolism in the heart.

Detail 65: The method of detail 61, wherein the contacting mitigates effects of TGF-Beta in the heart, wherein TGF-Beta is a growth factor associated with fibrosis.

Detail 66: The method of detail 61, wherein the 3D microtissues are derived from human mesenchymal stem cells.

Detail 67: The method of detail 61, wherein the contacting alters expression of one or more genes selected from the group consisting of: NPPA, NPPB, MYH6, MYH7, ACTA1, TNNT2, TNNC1, TNNI3, TPM1, MYL2, MYL3, MYL7, ACTC1, TTN, MYBPC3, MYH7B, MYL4, MYL5, MYL9, MYL10, MYL12A, MYL12B, MYLK, MYLK2, MYLK3, MYLK4, MYLPF, MYOM1, MYOM2, MYOM3, MYOT, MYOZ1, MYOZ2, MYOZ3, MYPN, MYOD1, MYOG, MYF5, MYF6, MYOCD, MYOC, MYOF, MYOM1, MYOM2, MYOM3, MYOT, MYOZ1, MYOZ2, MYOZ3, MYPN, MYOD1, MYOG, MYF5, MYF6, MYOCD, MYOC, MYOF, ACTN1, ACTN2, ACTN3, ACTN4, CAPN1, CAPN2, CAPN3, CAPN5, CAPN6, CAPN7, CAPN8, CAPN9, CAPN10, CAPN11, CAPN12, CAPN13, CAPN14, CAPN15, CAST, CAPNS1, CAPNS2, CASQ1, CASQ2, CASQ2P1, CASQ2P2, CASQ2P3, CASQ2P4, CASQ2P5, CASQ2P6, CASQ2P7, CASQ2P8, CASQ2P9, CASQ2P10, CASQ2P11, CASQ2P12, CASQ2P13, CASQ2P14, CASQ2P15, CASQ2P16, CASQ2P17, CASQ2P18, CASQ2P19, CASQ2P20, CASQ2P21, CASQ2P22, CASQ2P23, CASQ2P24, CASQ2P25, CASQ2P26, CASQ2P27, CASQ2P28, CASQ2P29, CASQ2P30, CASQ2P31, CASQ2P32, CASQ2P33, CASQ2P34, CASQ2P35, CASQ2P36, CASQ2P37, CASQ2P38, CASQ2P39, CASQ2P40, CASQ2P41, CASQ2P42, CASQ2P43, CASQ2P44, CASQ2P45, CASQ2P46, CASQ2P47, CASQ2P48, CASQ2P49, CASQ2P50, CASQ2P51, CASQ2P52, CASQ2P53, CASQ2P54, CASQ2P55, CASQ2P56, CASQ2P57, CASQ2P58, CASQ2P59, CASQ2P60, CASQ2P61, CASQ2P62, CASQ2P63, CASQ2P64, CASQ2P65, CASQ2P66, CASQ2P67, CASQ2P68, CASQ2P69, CASQ2P70, CASQ2P71, CASQ2P72, CASQ2P73, CASQ2P74, CASQ2P75, CASQ2P76, CASQ2P77, CASQ2P78, CASQ2P79, CASQ2P80, CASQ2P81, CASQ2P82, CASQ2P83, CASQ2P84, CASQ2P85, CASQ2P86, CASQ2P87, CASQ2P88, CASQ2P89, CASQ2P90, CASQ2P91, CASQ2P92, CASQ2P93, CASQ2P94, CASQ2P95, CASQ2P96, CASQ2P97, CASQ2P98, CASQ2P99, and CASQ2P100.

Detail 68: The method of detail 61, wherein the contacting alters expression of one or more genes selected from the group consisting of: ACTA2, ACTB, AGRN, ALB, ANXA1, ANXA11, ANXA2, ANXA5, ANXA6, BGN, BMP1, CFL1, COL11A2, COL14A1, COL18A1, COL1A1, COL1A2, COL2A1, COL3A1, COL4A1, COL4A2, COL5A1, COL5A2, COL5A3, COL6A1, COL6A2, COL6A3, COL9A1, COL9A2, CSTA, CSTB, CTSB, CTSD, DCN, EEF1A1, EEF2, EIF4A1, FBLN1, FBN1, FLG2, FN1, GAPDH, FLG, H1-4, H1-5, H2AC11, H2BC14, H2BC20P, H3C1, H4C1, HNRNPC, HNRNPK, HNRNPU, HSP90AB1, HSP90B1, HSPA5, HSPA8, HSPB1, HSPG2, KRT10, KRT14, KRT2, KRT5, LAMB1, LAMC1, LGALS3, LTBP1, LUM, MMP14, MMP2, MYH9, and NID2.

Detail 69: The method of detail 61, wherein the contacting alters expression of one or more genes selected from the group consisting of: P4HA1, P4HA2, PFN1, PKM, PLOD1, PLOD2, PLOD3, POSTN, PXDN, RAN, RPL11, RPS16, RPS25, RPS27A, RPS3, RPS8, RPSA, S100A10, S100A11, S100A14, S100A16, S100A4, S100A6, S100A8, S100A9, SERPINH1, SPARC, TGFBI, TGM1, TGM2, THBS1, TUBA1A, TUBB, TUBB3, TUBB4B, and VCAN.

Detail 70: A method of treating a diseased heart, comprising: administering to the diseased heart extra-cellular matrix (ECM) particles produced from decellularized 3D microtissues, wherein the administering treats the diseased heart by altering the proteome of the diseased heart.

Detail 71: The method of detail 70, wherein the administering restores a normal ratio of levels of different collagens in the heart.

Detail 72: The method of detail 70, wherein the administering alters an immune response in the heart.

Detail 73: The method of detail 70, wherein the administering alters metabolism in the heart.

Detail 74: The method of detail 70, wherein the administering mitigates effects of TGF-Beta in the heart, wherein TGF-Beta is a growth factor associated with fibrosis.

Detail 75: The method of detail 70, wherein the 3D microtissues are derived from human mesenchymal stem cells.

Detail 76: Extra-cellular matrix (ECM) particles for altering the proteome of a diseased heart, wherein the ECM particles are produced from decellularized 3D microtissues and treated to improve one or more of crosslinking, stability, injectability, or compatibility with a patient.

Detail 77: The ECM particles of detail 76, wherein the ECM particles, when contacted with the diseased heart, restore a normal ratio of levels of different collagens in the heart.

Detail 78: The ECM particles of detail 76, wherein the ECM particles, when contacted with the diseased heart, alter an immune response in the heart.

Detail 79: The ECM particles of detail 76, wherein the ECM particles, when contacted with the diseased heart, alter metabolism in the heart.

Detail 80: The ECM particles of detail 76, wherein the ECM particles, when contacted with the diseased heart, mitigate effects of TGF-Beta in the heart, wherein TGF-Beta is a growth factor associated with fibrosis.

Detail 81: The ECM particles of detail 76, wherein the 3D microtissues are derived from human mesenchymal stem cells.

Detail 82: A composition for treating a diseased heart, comprising: extra-cellular matrix (ECM) particles produced from decellularized 3D microtissues and treated to improve one or more of crosslinking, stability, injectability, or compatibility with a patient; wherein the composition, when administered to the diseased heart, treats the diseased heart by altering the proteome of the diseased heart.

Detail 83: The composition of detail 82, wherein the 3D microtissues are derived from human mesenchymal stem cells.

It is important to note that any method disclosed herein can be claimed as a composition and vice versa. At any point that a composition supports a “comprising” or an inclusion, a “consisting of” can be used to exclude additional ingredients. The description of embodiments of the disclosure is not intended to be exhaustive or to limit the disclosure to the precise form disclosed.

While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. Although methods and materials similar or equivalent to those described herein can be used in the practice or testing of this disclosure, suitable methods and materials are described below.

### EXAMPLES

### Example 1: An Exemplary Method of Making Customized Decellularized Human ECM Morsels

### Materials and Methods: Cell Culture, Micro-Mold Fabrication, and Formation of Microtissues (Single Multi-Cellular Structures).

Human lung fibroblasts (LF, ATCC CRL-4058) were expanded in Fibroblast Basal Medium (ATCC PCS-201-030) supplemented with Fibroblast Growth Kit-Low Serum (ATCC-201-041) and puromycin (Gibco A1113803) at a concentration of 0.3 μg/mL, treated with or without recombinant human transforming growth factor beta 1 (TGF-β1) protein (R&D systems, Minneapolis, MN, 240-B) at 0.625 to 10 ng/ml. Human lung fibroblasts from idiopathic fibrosis patient (IPF, ATCC PCS-201-020) were expanded in Fibroblast Basal Medium (ATCC PCS-201-030) supplemented with Fibroblast Growth Kit-Low Serum (ATCC-201-041), treated with or without recombinant human TGF-β1 protein (R&D systems, 240-B) at 0.625 to 10 ng/mL.

Human lung fibroblasts from patient with chronic obstructive pulmonary disease (COPD, ATCC PCS-201-017) were expanded in Fibroblast Basal Medium (ATCC PCS-201-030) supplemented with Fibroblast Growth Kit-Low Serum (ATCC-201-041). Human cardiac fibroblasts (HCF; Promocell C-12375) were expanded in Fibroblast Growth Medium 3 (C-23025). Human cardiac myocytes (HCM; Promocell C-12810) were expanded in Myocyte Growth Medium 3 (C-22070). Human cardiac microvascular endothelial cells (HCMEC; Promocell C-12285) were expanded in Endothelial Cell Growth Medium MV (C-22020). Human fetal cardiac fibroblasts (HFCF, Cell Applications Inc., San Diego, CA, 306-05f) were expanded in HCF Growth Medium (Cell Applications 316-500). Cells were trypsinized, counted, and re-suspended to the desired cell density for each experiment.

The inventors cast agarose gels from 3D Petri Dish® micro-molds (Microtissues, Inc., Providence, RI, USA) as previously described by Napolitano et al. (2007) Biotechniques 43 (4): 494, 496-500. Agarose gels were made with powdered agarose (Low-EEO/Multi-Purpose/Molecular Biology Grade, Fisher BioReagents, Thermo Fisher Scientific) sterilized by autoclaving and then dissolved in sterile phosphate buffered saline (PBS, HyClone SH30256.FS) to 1.5-2% (weight/volume). Micro-molds with round micro-wells were used to create spheroid-shaped microtissues. Round micro-wells for spheroids were 400 to 800 μm in diameter and contained either 35, 96, or 256 micro-wells per gel.

Additionally or alternatively, one of skill in the tissue engineering art could use computer-assisted design (e.g., Solid Works, Concord, MA) to create a template of the desired gel features (e.g., a cell seeding chamber, 721 micro-wells with hemispherical bottoms, 800 μm deep×600 μm wide). Then, one can generate a negative plastic mold with a prototyping machine (e.g., composed of acrylonitrile butadiene styrene (ABS) plastic (Protolabs)). Next, one can fill the negatives (e.g., with silicone rubber compound; MOLDMAX™ 25, Smooth-On, Macungie, PA) to produce positive replicates. The positive replicates are washed (e.g., with 70% ethanol, then rinsed with distilled water) and autoclaved before use. Then, one of ordinary skill in the tissue engineering can cast agarose gel with micro-wells directly from silicone molds, e.g., according to the methods of Napolitano et al. (2007) Biotechniques 43 (4): 494, 496-500, whereby 4 mL aliquot of molten 1.5-2% agarose-PBS solution is pipetted into each silicone mold in a sterile environment.

Agarose gels with micro-wells were seeded with trypsinized and counted cells at a density of 500 to 4,000 cells per spheroid micro-well. Cells aggregated in each of the micro-wells within the agarose gels, and self-assembled into 1 spheroid-shaped microtissue per micro-well within 4 to 24 hours after cell-seeding.

Cells within the micro-wells were cultured for 3, 6, 9 or 12 days after cell-seeding in a humidified incubator with 5% CO₂ and at 37° C., with media change every 3 days.

Visual inspection of microtissues: Microtissues in agarose gels were inspected with inverted light microscopy fitted with camera (e.g. Nikon Ti2, Zeiss Axio Observer Z1 or similar) to examine the size of the microtissues. The cross-sectional area of microtissues were measured using ImageJ (US National Institutes of Health, Bethesda, MD).

Examine histology of microtissues: Microtissues cultured for different days were fixed in 10% buffered formalin (Fisher 427098) in the agarose gels, paraffin-embedded, sectioned at 5 μm then stained with hematoxylin and eosin (H&E) or SIRIUS RED™ (Polyscience, Warrington, PA, 24901-250) to examine microtissue morphology or fibrillar collagen deposition, respectively.

### Examine Fibrillar Collagen Structure Microtissues.

Microtissues cultured for different days were fixed in 10% buffered formalin (Fisher 427098) in the agarose gels. Fibrillar collagen was visualized using an Olympus FV-1000-MPE multiphoton microscope (Olympus, Tokyo, Japan) equipped with a Mai Tai HP tunable laser with the excitation wavelength set to 790 nm and a 405/40 filter cube to select for fibrillar collagen second-harmonic signal. Microtissues fixed in 10% buffered formalin imaged in the agarose gel with a 25× (Numerical Aperture 1.05, Working Distance 2 mm) dipping objective in PBS.

### Examine the Proteomics of Microtissues.

Microtissues in agarose gels were washed with PBS three times then collected into a tube. ECM-enrichment and proteomics procedures as previously described by Naba et al. (2015) J Vis Exp, 2015 (101): p. e53057 were used to decellularize the microtissue and concentrate ECM proteins for proteomics analysis of the microtissues. Mass spectrometry by data dependent acquisition (DDA) and data analysis with Proteome Discoverer 2.3 (1% FDR) were used for the proteomics analysis of ECM proteins. An established iBAQ algorithm as described by Schwanhausser et al. (2011) Nature, 2011. 473 (7347): p. 337-42 was used to semi-quantity ECM components (by % molar of total ECM proteins) by dividing each individual protein's total intensity with the theoretical number of tryptic peptides between 6 and 30 amino acids in length (PeptideMass, SIB Swiss Institute of Bioinformatics).

### Decellularization of Microtissues.

Microtissues in agarose gels were washed with PBS three times. Decellularization of microtissues were either completed with microtissues remaining in the agarose gels, or after microtissues were collected into a tube. Microtissues in gels or in tubes were first treated with three rounds of 0.5% Triton-X100 (MilliporeSigma, St Louis, MO, T9284) in 20 mM NH₄OH (MilliporeSigma, 09859) in sterile PBS with protease inhibitors (PI; ThermoFisher Scientific, PI78439) for 45 mins with 60 rpm rotation per incubation, followed by three rounds of washes with sterile PBS+PI for 45 mins with 60 rpm rotation per incubation, then subjected to 1 round of incubation with DNase I (MilliporeSigma, 4716728001)+RNase A (Qiagen, Hilden, Germany, 19101)+PI for 72 hours with 60 rpm rotation, followed by three rounds of washes with sterile PBS+PI for 45 mins with 60 rpm rotation per incubation. The resulting decellularized microtissue ECM morsels were stored in sterile PBS at 4° C. for visual inspection and mechanical testing, fixed in 10% buffered formalin for histological analysis, or snap-frozen in liquid nitrogen and stored at −80° C. for subsequent biochemical analysis.

Visual inspection of decellularized microtissue ECM morsels: Decellularized microtissue ECM morsels in agarose gels were inspected with inverted light microscopy fitted with a camera as previously described to examine the size and architecture of the decellularized microtissue ECM morsels. Decellularized microtissue ECM morsels in tubes in sterile PBS were vortexed then photographed with iPhone, or transferred to a sterile 24-well plate (Corning, NY, 3527) then imaged with inverted light microscopy as previously described.

Examine histology of decellularized microtissue ECM morsels: Decellularized microtissue ECM morsels in agarose gels were fixed in 10% buffered formalin (Fisher 427098) in the agarose gels, paraffin-embedded, sectioned at 5 μm then stained with hematoxylin and eosin (H&E) or SIRIUS RED™ (Polyscience, Warrington, PA, 24901-250) to examine the presence of cell nuclei or fibrillar collagen deposition of decellularized microtissue ECM morsels, respectively.

Examine dsDNA Concentration of Decellularized Microtissue ECM Morsels.

DsDNA concentration of decellularized microtissue ECM morsels were measured as previously described by Blaheta et al. (1998) J Immunol Methods 1998 Feb. 1; 211 (1-2): 159-69. Decellularized microtissue ECM morsels that were collected into a tube were digested in papain solution (MilliporeSigma, P4762, 125 g/mL) in a sonicator for 72 hours at 65° C. The dsDNA concentration of the digested ECM was measured using QUANT-IT™ PICOGREEN™ dsDNA Assay Kit (Thermo Fisher Scientific, P7589) per manufacture's protocol.

### Examine Collagen Content of Microtissues.

Collagen content of microtissues was measured as previously described by Cissell et. al (2017) Tissue Eng Part C Methods 2017 April; 23 (4): 243-250. Microtissues were fixed in 10% formalin and stored at 4° C. until further processed. Fixed microtissues were collected into a tube and washed three times with 1×PBS, then digested in papain solution (MilliporeSigma, P4762, 125 μg/mL) in a sonicator for 10 days at 65° C. The digested microtissues was measured using a modified hydroxyproline assay as described by Cissell et al. (2017).

Examine Sulphated Glycosaminoglycans (sGAG) Content of Microtissues.

sGAG content of microtissues was measured using the 1,9-dimethylmethylene blue (DMMB) assay as described by Farndale et al. (1982) Connect Tissue Res 9 (4): 247-248, and Whitley et al. (1989) Clin Chem 35 (3): 374-379. Microtissues were fixed in 10% formalin and stored at 4° C. until further processed. Fixed microtissues were collected into a tube and washed three times with 1×PBS, then digested in papain solution (MilliporeSigma, P4762, 125 μg/mL) in a sonicator for 10 days at 65° C. The digested microtissues was measured using the DMMB assay as described by Farndale et al. (1982) and Whitley et al. (1989).

### Examine the Proteomics of Decellularized Microtissue ECM Morsels.

Decellularized microtissue ECM morsels that were collected into a tube underwent proteomics procedures as previously described by Naba et al. (2015) J Vis Exp, 2015 (101): p. e53057 for proteomics analysis of the microtissues. Mass spectrometry by data dependent acquisition (DDA) and data analysis with Proteome Discoverer 2.3 (1% FDR) were used for the proteomics analysis of ECM proteins. An established iBAQ algorithm as described by Schwanhausser et al. (2011) Nature, 2011. 473 (7347): p. 337-42 was used to semi-quantity ECM components (by % molar of total ECM proteins) by dividing each individual protein's total intensity with the theoretical number of tryptic peptides between 6 and 30 amino acids in length (PeptideMass, SIB Swiss Institute of Bioinformatics).

### Examine Mechanical Stiffness of ECM Morsels.

Samples tested went through a decellularization process, where plated on collagen-coated coverslips and incubated on the coverslips at 4° C. for 48 hrs prior to testing. Force measurements were collected using an atomic force microscope (AFM, MFP-3D-BIO, Asylum Research, Santa Barbara, CA) connected to a Nikon Eclipse Ti—U epifluorescence microscope (Nikon, Chicago, IL). The cantilever used had a spring constant of 0.03 N/m. Multiple testing sessions were conducted for the various samples to account for systematic errors. Force versus indentation data were analyzed using custom MATLAB scripts (The MathWorks, Natick, MA) utilizing the Hertz contact model.

All experiments were carried out at room temperature in fluid environments. The AFM was allowed to equilibrate before tests to minimize deflection laser and/or piezo drift. Force maps were collected for a variety of samples using a force mapping technique in contact mode. In brief, individual force curves were taken at discrete points across a region of interest. During analysis, the spatial arrangement of the data was retained to create a matrix of elastic modulus values. Force-indentation data were sampled at 5 kHz with an approach velocity of 10 μm/sec. A trigger force of about 4 nN was used for all samples with the deflection set to 100 nM. Scan size used was 5 μm and the resolution was 4×4 pts.

### Test Injectability of ECM Morsels.

Fetal cardiac microtissues were collected in a single tube and decellularized. The decellularized cultured fetal heart ECM in tube was imaged with a camera after vortexing (FIG. 6A). The cultured fetal heart ECM in tube were then passed through a 27-gauge syringe (inner diameter 0.21 mm) 10 times, then imaged the cultured ECM in tube again after syringe passage.

Some of the cultured fetal heart ECM were transferred with a sterile transfer pipet (with wide opening) into a clean well in 24-well plate lined with a thin layer of 2% (w/v) agarose for imaging under a Nikon microscope with a 10× objective (FIG. 6B). To mimic the surgical cultured ECM injection process, we removed the plunger of a 1 mL sterile syringe then transferred the cultured fetal heart ECM with a sterile transfer pipet into the open end of a 1 mL syringe connected to a 27-gauge needle. The syringe plunger was then placed back onto the opening end of the syringe containing the cultured ECM. Culture fetal heart ECM was then passed through a 27-gauge needle once directly into an unused well in the 24-well plate lined with a thin layer of 2% (w/v) agarose for imaging under a Nikon microscope with a 10× objective (FIG. 6B).

### Examine In Vitro Biocompatibility of ECM Morsels.

Decellularized adult or fetal heart ECM morsels were seeded with HCM or HCMEC and incubated for 24 hours. To examine proliferation, the nucleoside analog EdU (5-ethynyl-2′-deoxyuridine) was added 24 hours after cell seeding. Cells on ECM morsels in EdU were cultured for another 24 hours (HCMEC) or 48 hours (HCM), then fixed in 10% formalin for immunohistochemical evaluation using the Click-iT™ EdU Cell Proliferation Kit (Thermo Fisher Scientific) according to the manufacturer's protocol.

### Example 2

Results: Spheroid-Shaped ECM Microtissues Made with Different Human Cells Deposit ECM.

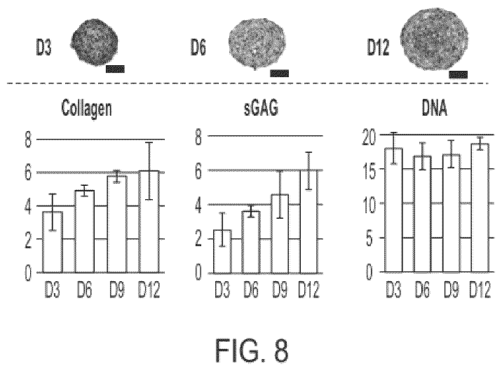

Eight different types of human microtissues were generated from human lung fibroblasts (LF) with or without TGF-β1, fibrotic human lung fibroblasts (IPF) with or without TGF-β1, COPD human lung fibroblast (COPD), adult human cardiac fibroblasts (HCF), fetal human cardiac fibroblasts (HFCF), tri-culture of HCF with human cardiac myocytes (HCM) and human cardiac microvascular endothelial cells (HCMEC), and tri-culture of HFCF, HCM and HCMEC as previously described. FIG. 4 illustrates eight out of nine different types of ECM microtissues in brightfield six days after seeding, and FIG. 7 illustrates microtissue sections stained with H&E and SIRIUS RED™ for the presence of fibrillar collagen (a common type of ECM) in all eight ECM microtissues. FIG. 7B and FIG. 8 illustrate that human fetal heart but not adult heart microtissues grew in size, had increased fibrillar collagen deposition and exhibited greater fibrillar collagen remodeling over culturing time.

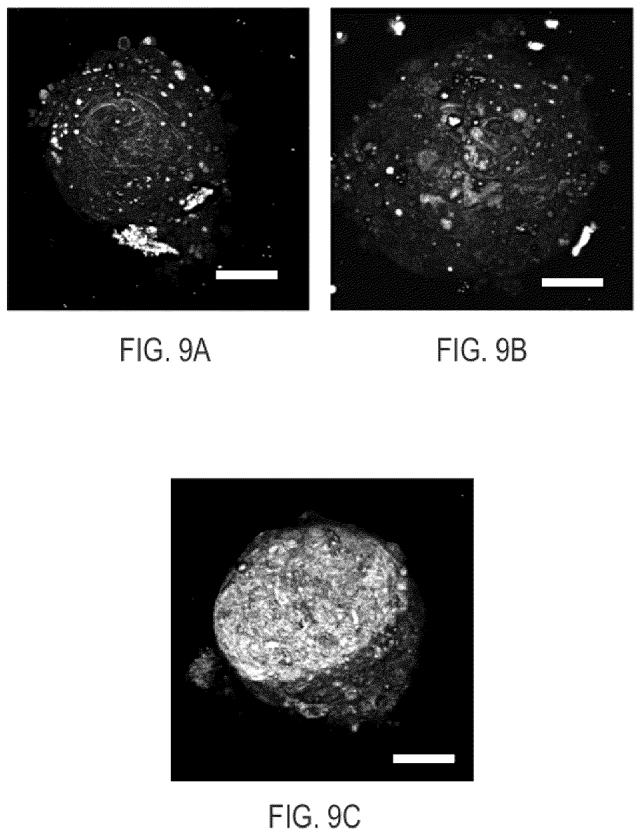

FIG. 9 shows human adult healthy lung ECM microtissues (FIG. 9A) had different fibrillar collagen structure than human fibrotic lung (FIG. 9B) and human adult healthy heart ECM microtissues (FIG. 9C) under multi-photon confocal second-harmonic generation (SHG) microscopy.

Proteomics analysis showed that ECM protein compositions microtissues were dependent on cell types used to make the microtissues.

After ECM-enrichment of microtissues, proteomics was performed to analyze the ECM composition of the various human ECM microtissues. Table 1 displays the percentage of each class of proteins for seven of the nine types of human ECM microtissues.

TABLE 1
Proteome Analysis of Seven Different Human ECM microtissues

| | | | | Healthy | | Healthy | |
|---|---|---|---|---|---|---|---|
| | Healthy | Fibrotic | COPD | Adult Heart - | Healthy | Fetal Heart - | Healthy |
| | Adult | Adult | Adult | Fibroblasts | Adult Heart - | Fibroblasts | Fetal Heart - |
| Protein | Lung | Lung | Lung | only | Triculture | only | Triculture |
| Class | (%) | (%) | (%) | (%) | (%) | (%) | (%) |
| Collagen | 29.59 | 9.93 | 17.39 | 3.99 | 2.90 | 7.53 | 4.30 |
| ECM | 2.46 | 4.46 | 5.11 | 6.49 | 7.02 | 6.28 | 7.13 |
| Regulators | | | | | | | |
| ECM-Affiliated | 15.44 | 24.43 | 22.42 | 21.86 | 26.93 | 18.24 | 28.18 |
| Proteins | | | | | | | |
| Glycoproteins | 49.06 | 48.67 | 41.73 | 40.42 | 35.14 | 58.38 | 40.38 |
| Proteoglycans | 0.42 | 0.96 | 1.79 | 3.65 | 2.77 | 2.05 | 1.29 |
| Secreted | 3.04 | 11.56 | 11.56 | 23.59 | 25.24 | 7.52 | 18.72 |
| Factors | | | | | | | |

The collagen and sGAG content of human ECM microtissues are tissue and age specific. After digestion of ECM microtissues, collagen and sGAG contents were evaluated using a modified hydroxyproline assay and the DMMB assay, respectively. Table 2 displays the collagen and sGAG content in microgram per one million cells of spheroid microtissues for five of the eight types of human ECM microtissues.

TABLE 2
Collagen and sGAG content (μg/10⁶ cells) of Five
Different Human ECM microtissues.

| | Collagen (μg) | sGAG (μg) |
|---|---|---|
| Healthy Adult Lung | 6.35 ± 2.42 | 2.83 ± 0.50 |
| Healthy Adult Lung + TGFβ treatment | 4.84 ± 0.82 | 4.81 ± 0.09 |
| Fibrotic Adult Lung | 2.87 ± 0.51 | 5.11 ± 1.68 |
| Healthy Adult Heart | 2.20 ± 0.17 | 6.16 ± 0.42 |
| Healthy Fetal Heart | 3.63 ± 1.68 | 4.79 ± 1.47 |

Spheroid-shaped microtissues can be decellularized in the agarose gel with micro-wells to efficiently remove cell nuclei while retaining ECM in morsels geometry. Six different types of decellularized human ECM morsels were generated from human LF with or without TGF-β1, IPF with or without TGF-β1, HCF, and HFCF as previously described. FIG. 4 illustrates the six different types of decellularized ECM morsels inside their individual micro-wells in brightfield six days after seeding, and FIG. 7 illustrates decellularized morsels sections stained with H&E showing the absences of cell nuclei, and SIRIUS RED™ for the presence of fibrillar collagen (a common type of ECM) post-decellularization in all six decellularized ECM morsels.

Spheroid-shaped microtissues collected into tubes then decellularized retained as morsels.

FIG. 5 illustrates that when fetal and adult human heart ECM spheroid microtissues were collected into one tube, decellularized, and mixed, the resulting decellularized fetal and adult human heart ECM morsels maintain their spheroid shape following the decellularization procedure without further processing.

Decellularized fetal human heart ECM morsels passed through 27G syringe 10 times without further processing FIG. 6.

Decellularized microtissue ECM morsels made with adult or fetal cardiac fibroblasts had less than 50 ng/mg ECM dry weight dsDNA.

The mechanical stiffness of decellularized microtissue ECM morsels are tissue and age specific.

Mechanical stiffness (elastic moduli) of decellularized microtissue ECM morsels were measured using AFM. FIG. 10 displays the elastic moduli in kPa for three of the nine types of human decellularized microtissue ECM morsels, the range for healthy human heart tissue cited by Zile M R et al. (2015) Circulation. 2015; 131(14): 1247-59 and Guimarães C F et al. (2020) Nature Reviews Materials. 2020; 5(5): 351-70, and the equivalent elastic modulus of injectable hydrogels that have been solidified (collagen, Matrigel, porcine cardiac ECM crosslinked with 0.1% glutaraldehyde or not) as cited in Singelyn J M et al. (2011) Macromol Biosci. 2011; 11(6): 731-8. Equivalent elastic modulus is 3 times the rheological measurements of storage moduli, assuming Poisson ratio v ˜0.5, as cited in Nemir S et al. (2010) Ann Biomed Eng. 2010; 38(1): 2-20.

### Cultured ECM is Biocompatible In Vitro.

To test their biocompatibility, we decellularized and washed the fetal cardiac microtissues while still inside their individual agarose microwells (in situ decellularization). The resulting decellularized cultured human fetal heart ECM stayed within the microwells (FIG. 11). Human cardiac myocytes (HCM) and cardiac microvascular endothelial cells (HCMEC) were then seeded onto the agarose mold. The cells settled by gravity onto the cultured ECM. The cells readily adhered and survived on the ECM (FIG. 11). The cells also exhibited greater proliferation than when cultured without ECM (FIG. 11).

Proteomics was then performed to analyze the ECM composition of the decellularized ECM morsels.

### Example 3

An ECM composition as disclosed herein is used in a method of treating MI. The composition is applied in the form of an aerosol to the affected area. The heart tissue regenerates within 16 weeks.

### Example 4

An ECM composition as disclosed herein is used in a method of treating MI. The composition is applied in the form of a patch to the affected area. The heart tissue regenerates within 12 weeks.

### Example 5

An ECM composition as disclosed herein is used in a method of treating MI. The composition is applied via injection to the affected area. The heart tissue regenerates within 24 weeks.

### Example 6

An ECM composition as disclosed herein is used in a method of treating a wound. The composition is applied via injection to the affected area. The tissue regenerates within 20 weeks.

### Example 7

An ECM composition as disclosed herein is used in a method of treating a wound. The composition is applied topically to the affected area. The tissue regenerates within 32 weeks.

### Example 8

An ECM composition as disclosed herein is used in a method of treating a wound. The composition is applied in the form of a patch to the affected area. The tissue regenerates within 16 weeks.

### Example 9

An ECM composition as disclosed herein is used in a method of treating a cosmetic condition. The composition is applied via injection to the desired treatment area.

In closing (Examples 1-9), it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

### Example 10. A Novel Method to Reproducibly Scale Up the Production of 3D ECM Particles from Human Stem Cells with a Consistent Composition of ECM

Human mesenchymal stem cells (MSCs) (Xeno-Free Human Umbilical Cord-Derived Mesenchymal Stem/Stromal Cells purchased from Rooster-Bio) were selected as a very important cell type. The cells were thawed and grown on tissue culture flasks. The cells were released from the flask by trypsinization, counted and seeded onto micro-molded agarose gels cast from a mold designed to scale up the process. Prior to seeding the cells, the agarose had been equilibrated with a selected cell culture medium. The MSCs formed 3D microtissues and 7 to 10 days later the MSC microtissues were decellularized by treatment with a mild detergent (Triton X100) followed by the enzyme DNase to remove DNA. This resulted in the production of MSC ECM particles. Three batches of MSC ECM particles were made (31,700, 39,960 and 44,280) each with an acceptably low level of DNA. Portions of the three batches of MSC ECM particles were sent to an outside lab (Omixs, Inc), and their matrisome analyzed by proteomics and shown to be highly consistent from batch to batch. The selection of MSCs is important because it is a stem cells and it is a cell source well known to the pharmaceutical industry which has already figured out how to scale up the production of these cells in 2D because they have been testing these cells as a living therapy in clinical trials. The ECM particles are made using human cells and so the ECM is entirely human. The ECM particles are made using a highly controlled industrial aseptic process that helps assure consistency and safety, of utmost importance to the FDA. These ECM particles are made using human stem cells and so we are able to make a unique formulation of the ECM, a formulation that cannot be found or extracted from cadaver or animal sources.

These MSC ECM particles have a wide variety of potential applications in the field of regenerative medicine such as the treatment of heart failure or other indications where scarring or fibrosis is problematic. These MSC ECM particles are products that could be sold as a biological therapy for a wide variety of indications.

### List of Materials and Equipment (Culture of MSCs)

Cell source: Xeno-free RoosterVial, Xeno-Free Human Umbilical Cord-Derived Mesenchymal Stem/Stromal Cells (hUC-MSCs), cells double approximately every 18 hours+/−4 hours depending on the donor. Cell diameter range: 12-15 μm (hUC) RoosterBiotech.

Cryopreserved cells 1×10⁶ cells, ($845/vial) (SKU: C43001UC). Cryopreserved cells 10×10⁶ cells, ($3,317/vial) (SKU: C43001UC). RoosterNourish-MSC-XF Xeno-free culture medium (SKU-KT-016) ($445) RoosterBiotech, contains:

RoosterBasal™-MSC 500 ml bottle (SU-005). RoosterBooster™-MSC-XF 10 mL bottle (SU-016). TrypLE Select Enzyme, trypsin containing solution for cell dissociation, $45.17 for 100 mL, $149.00 for 500 mL (12563029) Life Technologies. DPBS without calcium, magnesium (−/−) (14190250) $229.00/Case of ten 500 ml bottles, ThermoFisher. Vitronectin recombinant human protein (500 μg/mL) ThermoFisher. 1 mL (A14700) $72.50 per 1 mL. 10 mL (A31804) $696.00 per 10 mL. T-225 CellBIND flasks, Corning® CellBIND® 225 cm² Angled Neck Cell Culture Flask with Vent Cap (3293) ($442.00 per case, 25/Cs). T-150 CellBIND flasks, Corning® CellBIND® 150 cm² Angled Neck Cell Culture Flask with Vent Cap (3291) ($534.00 per case, 50/cs). Corning Costar 6-well Multiple-well cell culture plates with lid, treated, sterile, 5/pk, $447/case of 100, Fisher Scientific Corning #: 3506-Fisher #: 07-200-80.

### List of Materials and Equipment (Formation of Microtissues and ECM Particles)

Ultrapure Agarose, Fisher Bioreagents, (BP160-500), Molecular Biology Grade, Low EEO/Multipurpose, DNase, RNase not detected, $843/500 g Lot 178225.

Corning disposable vacuum filter system 1 L filter system, 0.2 μm cellulose acetate, case of 12 $443.00, Fisher Scientific Corning #430517-Fisher #: 09-761-40.

Large glass microscope slides 75×50 mm, Fisher 12-550C, box of 144, $78.20. Autoclaved before use. Small Polypropylene Instrument Trays Autoclavable-8.66″ L×5.91″ W×2.76″ Hgt., United States Plastic Corp, 76637, 2/pk, $19.52. Large Polypropylene Instrument Trays, Autoclavable-17.72″ L×5.91″ W×2.76″ Hgt., United States Plastic Corp, 76638, 2/pk, $27.41. ˜ 60 autoclavable, Custom designed silicone rubber molds to cast agarose gels with 721 microwells, custom made (FIG. 12A, FIG. 12B). Repeat Pipettor, Vista-Lab Technologies, ali-Q 2 VS, Variable aliquoting speed, 10-101-133, Fisher Scientific. Dulbecco's Modified Eagle's medium (DMEM) with high glucose, I-glutamine, phenol red, and sodium pyruvate (11995065, Thermo Fisher Scientific ($30.11 per 500 ml bottle). Advanced DMEM (12491015) (Thermo Fisher Scientific) ($42.30 per 500 mL bottle). Penicillin-Streptomycin stock (Pen/Strep), Dual Antibiotic Solution, MP Biomedicals 10,000 IU Pen and 10,000 IU Strep) ($47.16 per 100 mL) (091670249) Thomas Scientific). L-proline. 2-phospho-L-ascorbic acid trisodium salt. Glutamax, 100× stock ($44.10 per 100 mL) (35050061) Thermo Fisher Scientific. Phosphate buffered saline (1×), cytiva HyClone™ (SH30256.02). Halt Protease Inhibitor Cocktail, EDTA free, 100× stock, (PI78439) Thermo Fisher Scientific, $700. Triton X-100 (100 mL, $52.30) Millipore Sigma. 1M ammonium hydroxide (09859) Millipore-Sigma, $67.50/250 mL. 10× DNase buffer, New England Biolabs (NEB) (B0303S) $23.00 for 6 mL, supplied as a 10× stock. DNase I recombinant, RNase-free Sigma-Aldrich (04716728001) $202.00.

RNase A supplied as a stock of 100 mg/ml; 7000 units/ml, solution, Qiagen, (19101) (2.5 mL) $263.00. Normal Saline with 9.0 g/Liter Sodium Chloride, Quality Biological, Cat #114-055-101, Lot #724618, 500 mL. Human serum albumin, recombinant, expressed in rice, lyophilized powder, suitable for cell culture, low endotoxin, Sigma A9731-1G $126/gram. CryoStor CS5 Freeze Media, Bio Life Solution, Part #205102 100 mL for $425. Mr. Frosty™ Freezing Container, Catalog number: 5100-0001, Thermo Scientific. Pyrex glass bowl 3140-150 Brand 3140 dish; 150×75 mm, pack of 4, Cole-Parmer, CAT #UX-34550-06, $332.00 for pack of 4. Autoclaved before use.

Standard lab ice buckets. Orbi-Shaker CO₂ Benchmark All environment, Shaker. Mold Max® 25 Gallon Unit Net Wt: 11.57 lbs, Smooth-On, (cat #mold-max-25) used to make molds for molding agarose gels and custom designed shelf to hold gels when harvesting microtissues. 1250 ul Low Binding Racked Pipette Tips, Cat #24-865R, Genesee Scientific, 10 Racks of 96 tips/unit. Sterile. Protein LoBind Tube 2.0 mL, Eppendorf tubes, Cat #022431102. Sterile. ThermoForma Micro Centrifuge, Model #5522 MicroMax RF, Ser. No. 99/985,207.

Culture Media and Solutions; Serum Free Medium Plus (SFM) contains: 500 ml bottle of Dulbecco's Modified Eagle's medium (DMEM) with high glucose, I-glutamine, phenol red, and sodium pyruvate. Penicillin-Streptomycin. Add 5 mL of stock per 500 ml bottle. Cap and swirl bottle to mix. Label and store at 4° C.

Serum Free Medium Plus (SFM+) contains: 500 ml bottle of Dulbecco's Modified Eagle's medium (DMEM) with high glucose, I-glutamine, phenol red, and sodium pyruvate. L-proline. Add 25.3 mg of powder per 500 ml bottle. (final concentration is 50 μg/mL L-proline). 2-phospho-L-ascorbic acid trisodium salt. Add 16.3 mg of powder per 500 ml bottle. (final concentration is 0.1 mM). Penicillin-Streptomycin. Add 5 mL of stock per 500 ml bottle. Cap and swirl bottle to mix. Be sure powders have dissolved. Filter sterilize. Label and store at 4° C.

Serum Free Medium Advanced (SFMA) contains: 500 ml bottle of Advanced DMEM. Glutamax. Add 10 mL of the 100× stock per 500 ml bottle (final concentration is 4 mM). Penicillin-Streptomycin. Add 5 mL of stock per 500 ml bottle. Cap and swirl bottle to mix. Label and store at 4° C.

50:50 cell culture medium: Mix equal volumes of SFM+ and SFMA. Filter sterilize. Label and store at 4° C.

Phosphate buffered saline-protease inhibitor (PBS-PI) (you will need 40 mL) and it contains: 39.6 mL cold phosphate buffered saline. Refrigerate prior to use.

Halt Protease Inhibitor Cocktail. Add 0.4 mL of 100× stock. Add Halt Protease Inhibitor Cocktail to buffer immediately before use. Filter sterilize and keep cold.

Phosphate buffered saline-protease inhibitor with Triton X100 (PBS-PI+Triton X100) (you will need 300 ml) it contains: 289.5 mL cold phosphate buffered saline.

Triton X-100. Add 1.5 mL, final concentration is 0.5%. Note: Triton X-100 is very viscous and takes time to dissolve. Pipette up and down to be sure no Triton X-100 sticks to the pipette. Dissolve on shaker or on water bath. Ammonium hydroxide. Add 6 mL of a 1M stock. (final concentration is 20 mM ammonium hydroxide). Before use, chill this solution in the refrigerator. Halt Protease Inhibitor Cocktail. Add 3 mL of 100× stock. Add Halt Protease Inhibitor Cocktail to buffer immediately before use. Filter sterilize and keep cold.

DNA/RNA digestion solution (you will need 10 mL) and it contains: DNase buffer. Add 1 mL of a 10× stock. Milli Q water. Add 8.7 mL. DNase I, RNase-free. Add 200 uL of a 10,000 U/mL stock, (final concentration is 200 U/mL). RNase A. Add 2 uL of a 100 mg/mL (7000 units/mL) stock, (final concentration is 20 μg/mL). Halt Protease Inhibitor Cocktail. Add 100 uL of 100× stock just before use. Sterilize with a syringe fitted with a filter. Be careful to use disposable pipettes and a designated pipettor when working with RNAse. RNAse can create huge problems when working with the RNA needed for RT-PR analyses.

Human albumin 100× stock solution: Human albumin powder, Weigh out 200 mg. Dissolve human albumin in 20 mL of saline (final concentration is 10 mg/mL). Filter sterilize. Aliquot and keep frozen at −20° C.

Human albumin pre-coating solution: To 200 ml of saline add 2 mL of 100× human albumin 100× stock solution (final concentration is 0.1 mg/mL). Protocol for pre-coating tips, tubes and surfaces. NOTE: MSC ECM particles are very sticky and can result in unacceptable losses! All surfaces, tubes and tips (even LoBind) that come into contact with MSC ECM particles must be pre-coated with human albumin. Use this protocol to prevent sticking: Draw up into tip, fill tube, or cover surface with human albumin pre-coating solution. Wait 60 seconds. Wash 5 times with PBS.

Description of molds and theoretical yield of ECM particles: The mold shown in FIG. 12A is commercially available and is used to form a micro-molded agarose gel that fits in a 12 well dish. These molds were used in the prior experiments presented. This agarose gel will form 256 microtissues at a time. As a step towards scale up of the production of microtissues for the formation of ECM particles, we used Solidworks to design a mold to fit in a 6 well dish shown in the center. This custom mold produces a micro-molded agarose gel with 721 micro-wells. We designed a “master” mold to be used to make many working molds for 6 well plates. This master mold is shown in FIG. 12B.

Molds for making micro-molded agarose gels: The mold in FIG. 12A on left makes gel with 256 micro-wells. Each gel fits in one well of a 12-well plate. The custom mold shown at the right of FIG. 12A makes agarose gel with 721 micro-wells. Each gel fits in one well of a 6-well plate. The mold shown in FIG. 12B is a custom “master mold” designed to produce multiple copies of the mold for 6 well plates. Thus, the output of a multi-well plate (6-well) within the industry standard footprint of all multi-well plates (127.76 mm×85.48 mm) is increased to 4,326 microtissues per plate with significantly fewer handling steps. Thus, the theoretical yield of ECM particles from a large build of many 6 well plates equal the number of 6 well plates times 4,326 microtissues per plate.

Scale Up Cell Number: hUC-MSCs.

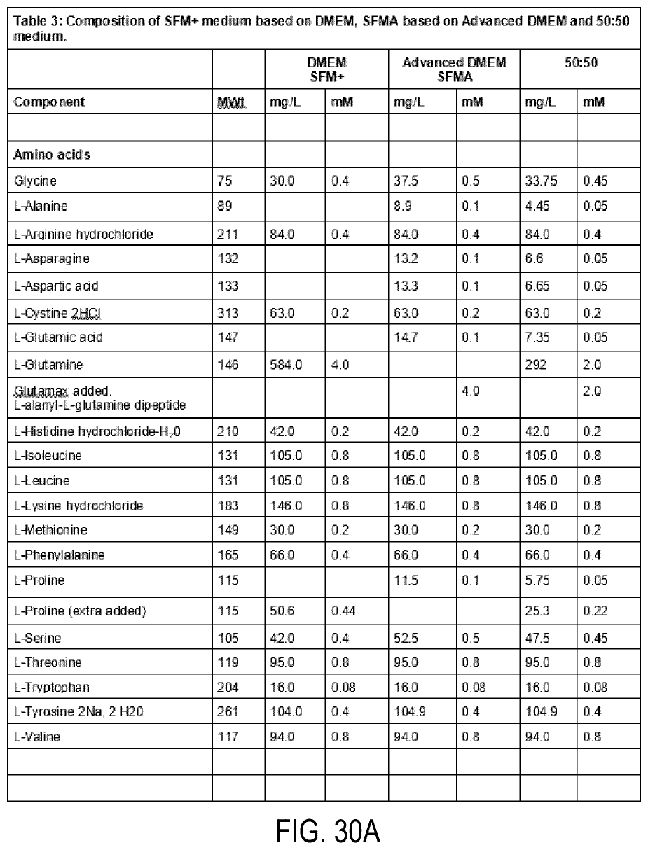

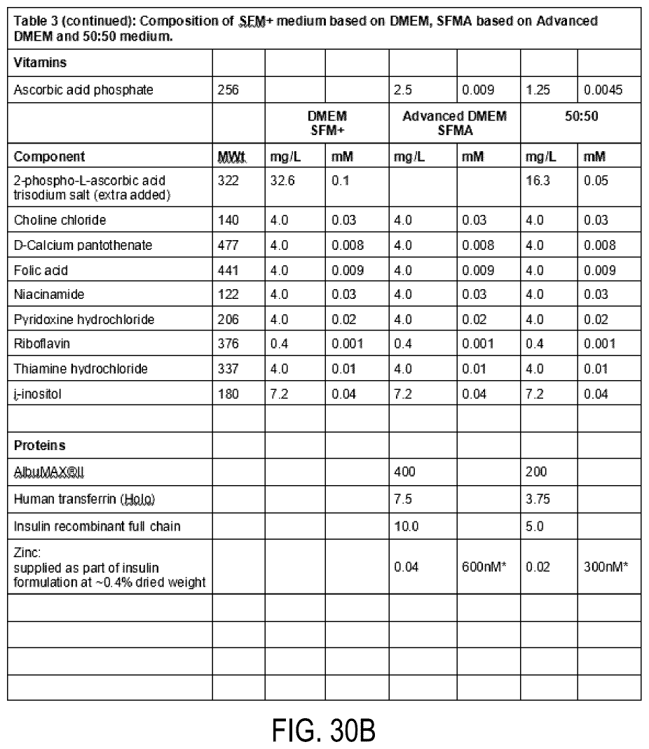

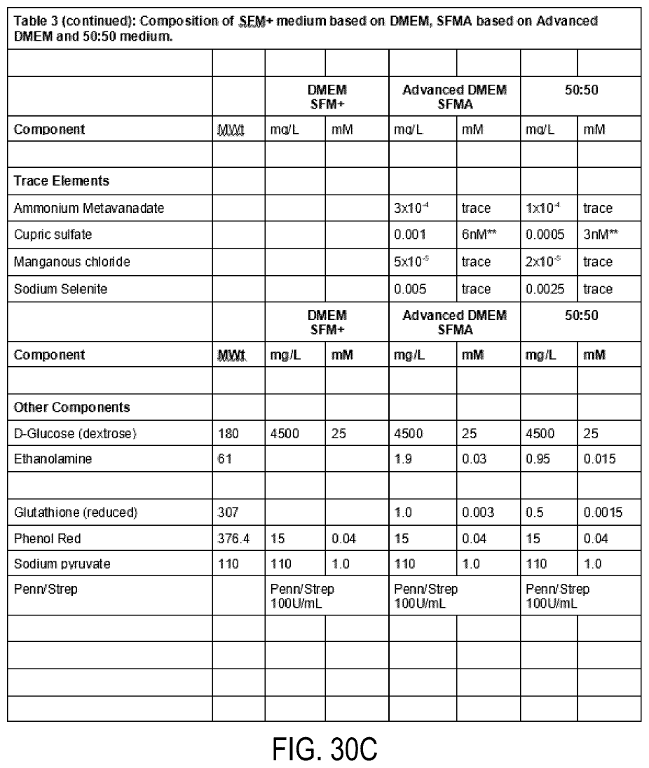

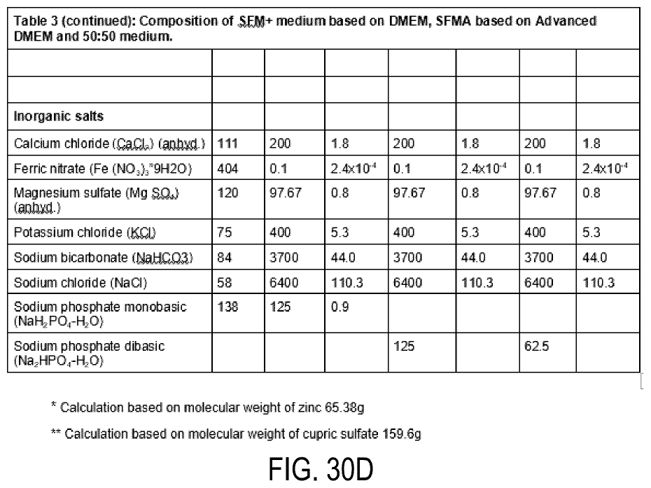

Before starting, a worksheet (not shown) was provided with this protocol to plan a calendar around key events in the protocol. FIG. 30A, FIG. 30B, FIG. 30C, and FIG. 30D show example components in a composition of SFM+ medium based on DMEM, SFMA based on Advanced DMEM and 50:50 medium.

Day 1 Preparation of MSC culture medium: Bring RoosterNourish-MSC-XF components to room temperature, protected from light, for up to four hours. (Use aluminum foil to protect from light). Prepare 1 bottle of medium by aseptically combining 1 bottle of RoosterBooster™-MSC-XF (Part No. SU-016) to 1 bottle of RoosterBasal™-MSC (Part No. SU-005/SU-022). Cap the bottle and gently mix. Coat flasks with vitronectin. Upon receipt, thaw the vial of vitronectin and transfer DPBS−/− to room temperature. Pipette 50 mL of DPBS−/− into each of two 50 mL tubes.

To each 50 ml tube, add 150 uL of vitronectin stock (500 ug/mL). Cap and invert tube to mix well. Make sterile. Final concentration of this dilution is 1.5 μg/mL. Target concentration for vitronectin coating is 0.15 ug/cm². T-225 flask requires 33.75 ug vitronectin. Adding 22.5 mL of 1.5 ug/mL to each T-225 flask is a total of 33.75 ug. To each of six T-150 CellBind flasks, add 15 mL of DPBS−/− with vitronectin. Incubate flasks at room temperature for at least 1 hour (away from direct light). Aliquot remaining vitronectin stock and return to −80° C. promptly. Aspirate volume within flasks (do not allow flasks to completely dry). Thaw MSCs: Aseptically transfer 20 mL of prepared medium into a 50 mL centrifuge tube. Thaw 2 vials of RoosterVial-hUC-1M-XF manually in a 37° C. water bath. When thawing in a water bath, monitor the vial closely and remove from water bath once only a small bit of ice is remaining (2-3 min). Aseptically transfer vials into a Biosafety Cabinet (BSC). Transfer vial contents into the 50 mL centrifuge tube containing prepared medium and mix cell suspension well. Centrifuge at 350× g for 6 minutes. Aspirate the supernatant and resuspend cells in 60 mL of RoosterNourish-MSC-XF medium. (Use two 50 mL tubes and spilt the 60 mL into two tubes). Mix well and seed 10 ml of cells into each of the six vitronectin coated T-150 CellBind flasks. Add 20 mL of medium to each flask. Transfer flasks into an incubator (37° C., 5% CO₂) and the ensure surfaces are covered evenly with medium.

Day 3: Begin monitoring cell growth. Microscopically monitor cell confluency starting on day 3 of culture. When culture is >80% confluent, cells are ready to harvest. Prepare 1 liter of serum free-medium to equilibrate gels (3 changes). Prepare 2 liters of 50:50 medium to be sure you have enough Day 4: Cast and equilibrate gels (can be done anywhere from day 2-4). Using large clean sterile molds designed to make 721 microtissues, add 4 mL of sterile 2% weight/volume (w/v) molten agarose solution in phosphate buffered saline (PBS). (250 ml of agarose+PBS, 5 g of agarose needed). Before covering with a glass slide use a spatula to ensure the bubbles are not trapped in-between the post. Cover mold with a sterile glass slide to flatten the agarose. After the agarose has solidified (˜15 minutes), gently separate gel from mold and transfer to a 6 well plate always using aseptic technique. Repeat this process until a total of 54 gels have been made (nine 6 well plates). To each well, add 4 mL of Serum Free Medium (SFM). (216 mL total). Transfer plates to the 37° C. incubator and incubate for ˜2 hours. Remove plates, aspirate off medium, add 4 mL of SFM to each well, return plates to the 37° C. incubator for overnight incubation. (216 mL total).

Day 5: Harvest cells and freezing down extra cells. Before you start harvesting cells, remove the 6 well plates with gels, aspirate off medium, add 4 mL of SFM to each well, return plates to the 37° C. until you are ready to start the cell seeding process. (216 mL total). For cell harvest, transfer vessels into biosafety cabinet and remove spent medium. Add 6.67 mL TrypLE to each flask. Distribute TrypLE evenly to cover all the cells and place vessels in 37° C. (5% CO₂) incubator. Check culture every 5 minutes until cells are detached from surface. Gently tap to dislodge the remaining cells from surface. Add 6.67 mL of RoosterNourish™-MSC-XF media to each flask to stop the TrypLE activity. Transfer the cell suspension into a 50 mL centrifuge tube. (Step 32-34 will give you 80 mL. Put into two 50 mL tubes ˜40 mL in each tube). Centrifuge at 350×g for 6 minutes. Aspirate the supernatant. RoosterBio estimates the total cell number of six T-150 CellBind flasks to be a total of about 50M, but this needs to be determined. (Highlighted numbers will be changed depending on cell concentration). Count cells and record cell concentration as well as the total number of cells. Pipette 10 uL of cells into hemocytometer to count cells. Use Worksheet 2 to record data and calculations. If you have extra cells for freezing. See Protocol: Cryopreservation of MSCs. Resuspend cells in 50/50 medium to achieve desired cell concentration for seeding gels. Dilute or re-centrifuge to get desired concentration. Seed gels with cells to form microtissues Just before you are ready to start the cell seeding process, remove the 6 well plates, aspirate off the medium from around the gel and carefully remove the medium from the seeding chamber without damaging the gel. Resuspend cells in 50:50 medium to a concentration of 721,000 cells/mL (target 1,000 cells/microtissue). You will need a total of at least 54 mL to seed 54 gels. Seeding Gels (Calculations for 60 gels) Gels are seeded with 721,000 cells/gel or 1,000 cells/micro-well for 721 micro-wells. 721,000 cells/well×60 gels=43,260,000 cells needed. Volume of cell suspension needed=total number of cells needed/cells per mL. 43,260,000 cells/1,592,500 cells/mL=27.16 mL of cells needed. Resuspension Volume 1×60=60 mL. Volume of medium to add=resuspension volume-volume cell suspension needed 60-27.16 mL=32.84 mL of medium needed. Add 1 mL of this cell suspension to the seeding chamber of each gel. Carefully distribute the drops around the gel to get uniform cell seeding. Typically seeding drop wise around the edge of the gel and finishing in the center to ensure all parts of the gel have cells. Allow plates to sit in the hood for 10 minutes before carefully transferring them to the 37° C. CO₂ incubator. After 20 minutes, carefully remove the plates from the incubator and carefully add 4 mL of 50:50 medium to the area around the gel. Pipette the 4 mL slowly down the side of the well to not disturb the middle of the gel where the cells are still settling. (216 mL total). Return plates to the 37° C. CO₂ incubator.

Day 6: Refresh 50:50 medium. Next day, remove plates, aspirate off medium, add 4 mL of 50:50 medium to each well, and return plates to the 37° C. incubator. (216 mL total needed).

Day 8. Refresh 50:50 medium.

Two days later, remove plates, aspirate off medium, add 4 mL of 50:50 medium to each well, return plates to the 37° C. incubator. (216 mL total needed).

Day 10. Refresh 50:50 medium.

Two days later, remove plates, aspirate off medium, add 4 mL of 50:50 medium to each well, and return plates to the 37° C. incubator. (216 mL total needed).

Day 12. Refresh 50:50 medium.

Two days later, remove plates, aspirate off medium, add 4 mL of 50:50 medium o each well, and return plates to the 37° C. incubator. (216 mL total needed).

Day 13. Harvest and decellularize microtissues

Set up one Pyrex glass bowl on ice. Create a pitch so that solutions will run down and pool at the bottom.

Place a sterile silicone rubber shelf at about the halfway mark of the bowl to hold gels in place and prevent them from sliding down the bowl.

Coat the bowl with a solution of 0.1 mg/mL BSA in saline for a minimum of 60 seconds and rinse with saline ×5.

Remove a 6 well plate, aspirate off medium from around each gel and carefully remove medium from the seeding chamber without damaging the gel or aspirating up the microtissues.

Use a spatula or a flat tip tweezer to transfer two gels to the shelf of each glass bowl.

(Because these spheroids tend to stick to any plastic surfaces, Iobind 2 mL Eppendorf tubes, 15 mL or 50 mL Savillex tubes and Iobind P1000 pipette tips will be used to have maximum product recovery)

Before using a p1000 Genesee Scientific coat the inside of the tip with the 0.1 mg/mL BSA in saline. Complete this step anytime you change pipette tips.

Place cold PBS-PI+Triton X100 into the bowl. Use a P1000 with a Iobind tip to dislodge the microtissues from each gel by squirting the cold PBS-PI+Triton X100. Approximately 6 or more squirts distributed evenly over the gel should be enough to dislodge all the microtissues.

Note this approximate number is not the same for every gel. Some of the spheroids may take more time to dislodge from the gels!

As each set of two gels is finished, transfer the solution with microtissues to a 2 mL Iobind Eppendorf tubes which has also been coated with 0.1 mg/mL and rinsed with saline on ice. Once in the tube, allow the microtissues to settle to the bottom of the tube. Check under the microscope to be certain microtissues have been removed from the gel. If needed, repeat squirting process with colder PBS-PI+Triton X100.

Once all microtissues have been removed from the gels, rinse the bottom of the glass bowl with some of the PBS-PI+Triton X100 to ensure that nearly all microtissues have been gathered. Once all microtissues have settled in the tubes, aspirate off PBS-PI+Triton X100 in each of the 2 mL Iobind Eppendorf tubes until each tube has 2 mL or more. Insure there is very little room at the time to prevent drying out. Pool all microtissues into five 2 mL tubes on ice. Use the Nageotte Counting Chamber to count spheroids and record data. See Protocol: Counting Spheroids and ECM particles. Place tubes in a micro-centrifuge and spin at 2,200 RPM×6 minutes. Aspirate off the PBS-PI+Triton X100 and resuspend the microtissues in 2 mL per each 2 ml tube with of PBS-PI+Triton X100. Incubate at 37° C. for 30 minutes (Wash 1). Place tubes in a micro-centrifuge and spin at 2,200 RPM×6 minutes to pellet the microtissues. Aspirate off the PBS-PI+Triton X100 and resuspend the microtissues in total 10 mL (2 mL×5 tubes) of PBS-PI+Triton X100. Incubate at 37° C. for 30 minutes (Wash 2). Place tubes in a micro-centrifuge and spin at 4,000 RPM×6 minutes to pellet the microtissues. Aspirate off the PBS-PI+Triton X100 and resuspend the microtissues in total of 5 mL of PBS-PI+Triton X100. Incubate at 37° C. for 30 minutes (Wash 3). Place tubes in a micro-centrifuge and spin at 4,000 RPM×6 minutes to pellet the microtissues. Aspirate off the PBS-PI+Triton X100 and resuspend the microtissues in total 10 mL of PBS-PI. Note: This solution, PBS-PI, does not have Triton ×100. Incubate at 37° C. for 30 minutes (Wash 4). Place tubes in a micro-centrifuge and spin at 4,000 RPM×6 minutes to pellet the microtissues. Aspirate off the PBS-PI and resuspend the microtissues in 10 ml of PBS-PI. Incubate at 37° C. for 30 minutes (Wash 5). Place tubes in a micro-centrifuge and spin at 4,000 RPM×6 minutes to pellet the microtissues. Aspirate off the PBS-PI+Triton X100 and resuspend the microtissues in 5 mL of PBS-PI. Incubate at 37° C. for 30 minutes (Wash 6). Place tubes in a micro-centrifuge and spin at 4,000 RPM×6 minutes to pellet the microtissues. Aspirate off the PBS-PI+Triton X100 and resuspend the microtissues in 10 ml of DNA/RNA digestion solution.

Secure all tubes and incubate at 37° C. for 72 hours. Come in every morning and turn the tubes upside down to all for the microtissues to disperse all throughout the tube. NOTE: Use disposable pipettes and a designated pipettor when working with RNAse. RNAse can create huge problems when working with the RNA needed for RT-PR analyses.

Day 16: Wash and store ECM particles. Place tubes in a micro-centrifuge and spin at 2,200 RPM×6 minutes to pellet the microtissues. Aspirate off the DNA/RNA digestion solution and resuspend the microtissues in 5 mL of saline suitable for injection. Incubate the tubes at 37° C. for 30 minutes (Wash 7).

All the microtissues to settle naturally and then aspirate off the saline and resuspend the microtissues in 10 mL of saline total.

Incubate the tubes at 37° C. for 30 minutes (Wash 8).

All the microtissues to settle naturally and then aspirate off the saline and resuspend the microtissues in 10 ml of saline total.

Incubate the tubes at 37° C. for 30 minutes (Wash 9).

All the microtissues to settle naturally and then aspirate off the saline and resuspend the microtissues in 10 ml of saline total.

Store the ECM particles in saline at 4° C.

Use the Nageotte Counting Chamber to count ECM particles and record data.

See Protocol: Counting Spheroids and ECM particles.

Day 17 onward: Characterize ECM particles.

Two detailed worksheets (not shown) were used for characterization.

### Example 11. A Novel Method to Treat Heart Failure by Injection of ECM Particles Produced from Decellularized 3D Microtissues of Human Mesenchymal Stem Cells

ECM particles produced from decellularized 3D microtissues of human mesenchymal stem cells were injected into a mouse model of heart failure and shown to have beneficial effects as measured by an increase in fractional shortening, a decrease in infarct scar size and an increase in capillaries/arterioles. These particles may be a method to treat heart failure and or other indications where scarring/fibrosis is problematic. These particles could be a product to treat disease. The treatment of heart failure and other indications where scarring and fibrosis are problematic are major unmet medical needs. ECM particles are potentially a new regenerative therapy to treat diseases whose underlying pathology is multi-factorial.

Objective: Extracellular matrix (ECM) has shown promise as a treatment for ischemic cardiac injury. Traditionally, ECM is obtained either by decellularizing donor animal tissue, or by bioengineering new ECM from stem cells. Unfortunately, both of these strategies have drawbacks: harvested ECM retains the superstructure of the original tissue, while bioengineered ECM lacks the hierarchical structure conferred by crosslinking and thus has diminished integrity. More recently the technology has been developed to use stem cell seeded micro-molds to form 3D microtissues that when decellularized create ECM particles that retain hierarchal architecture and are injectable. The purpose of this study was to investigate the potential of ECM particles in the treatment of myocardial ischemia.

Methods: ECM particles were suspended at a concentration of 63,400 particles per mL to create an injectable solution. FVB mice underwent left anterior descending (LAD) artery ligation followed by intramyocardial injection of 10 μL of saline control (n=10) or ECM solution (n=10) to the infarcted area. Echocardiography was performed pre-op and on post-op days 3, 7, 14, 21, and 28 in order to determine ejection fraction (EF) and fractional shortening (FS). On POD28, mice were euthanized, and cardiac tissue was collected for proteomic analysis, immunohistochemistry, and immunofluorescent microscopy.

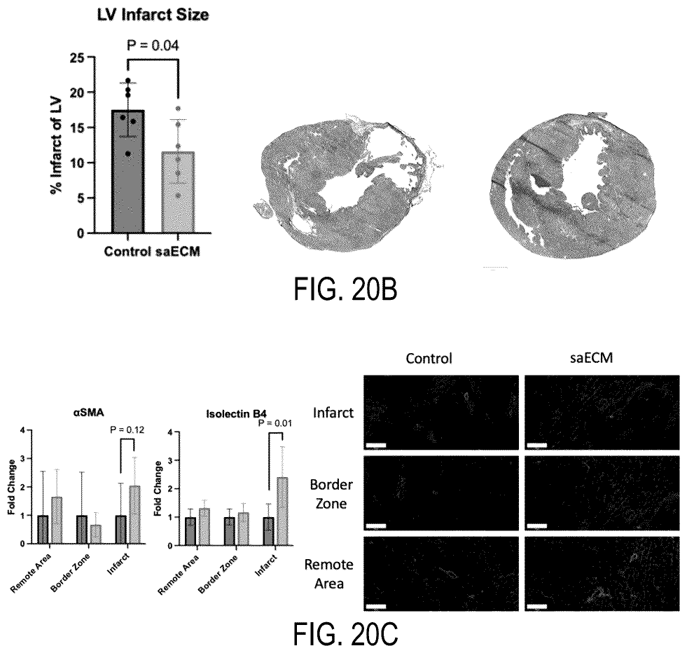

Results: Treatment with ECM particles resulted in significant improvement in FS after POD14 that continued to POD28 (p<0.05 for all). A similar trend in improved EF was observed, but failed to reach statistical significance (FIG. 20A). Infarct size was significantly reduced in ECM treated mice compared with controls (p=0.04) (FIG. 20B). Within the infarcted region there was a trend towards increased arteriolar density in ECM treated mice (p=0.12) and a significant increase in capillary density (p=0.01). No significant difference was observed in arteriolar or capillary density in the infarct border zone or remote non-ischemic area (p>0.05 for all) (FIG. 20C). No significant difference was observed in ki67, TUNEL, TGFß, or 3-nitrotyrosine staining in any heart region (p>0.05 for all).

Effects of saECM on cardiac tissue are shown in the data. Control injection samples are represented in grey and saECM particles treated hearts are represented in magenta (different shade of grey). FIG. 20A shows echocardiographic data pre-op and POD 3, 7, 14, 21, and 28. FIG. 20B shows analysis of percent LV infarct and representative images of Masson's trichrome stain. FIG. 20C shows duel immunofluorescence microscopy of isolectinB4 (endothelial cells) and αSMA (arterioles). Left panels show quantitative analysis of capillaries (isolectin B4) and arterioles (alpha-SMA) in control and saECM hearts (n=6).

Conclusion: Treatment with ECM particles resulted in decreased infarct size, improved FS, and increased vascular density in a murine model of myocardial ischemia. The ability to precisely control the construct of ECM particles represents a potential therapeutic strategy for the treatment of myocardial ischemia.

### Example 12. Additional (Optional) Methods of Making Customized MSC ECM Particles

An exemplary method of making customized human MSC ECM particles is illustrated.

### Materials and Methods

### Cell Culture, Micro-Mold Fabrication, and Formation of Microtissues (Single Multi-Cellular Structures)

MSC culture medium was prepared as follows. Briefly, human mesenchymal stem cells (MSCs) (Xeno-Free Human Umbilical Cord-Derived Mesenchymal Stem/Stromal Cells purchased from Rooster-Bio) were selected as the cell type. The cells were thawed and grown on tissue culture flasks. The cells were released from the flask by trypsinization, counted and seeded onto micro-molded agarose gels cast from a mold designed to scale up the process. Prior to seeding the cells, the agarose had been equilibrated with a selected cell culture medium. The MSCs formed 3D microtissues and 7 to 10 days later the MSC.

One bottle of medium was prepared at room temperature by aseptically combining one bottle of RoosterBooster™-MSC-XF (Roosterbio, Frederick, MD; Part No. SU-016) to 1 bottle of RoosterBasal™-MSC (Part No. SU-005/SU-022), capping the bottle and gently mixing.

Cell culture flasks were coated with vitronectin by thawing a vial of vitronectin stock (500 μg/mL) to room temperature. 50 mL of DPBS−/− was pipetted into each of two 50 mL tubes. To each 50 mL tube was added 150 μL of vitronectin stock. Tubes were capped and inverted to mix well and filter sterilized. The final concentration of this vitronectin dilution was 1.5 μg/mL. The target concentration for vitronectin coating was 0.15 μg/cm². A T-225 flask requires 33.75 μg vitronectin by adding 22.5 mL of 1.5 μg/mL to each T-225 flask. 15 mL of DPBS−/− with vitronectin was added to each of six T-150 CellBind flasks, which were incubated at room temperature for at least 1 hour (away from direct light). The volume within flasks was aspirated without allowing flasks to completely dry. 20 mL of prepared MSC culture medium was aseptically transferred into a 50 mL centrifuge tube.

Two vials of human mesenchymal stem cells (MSCs; Xeno-Free Human Umbilical Cord-Derived Mesenchymal Stem/Stromal Cells; Rooster-Bio, Frederick, MD) were thawed manually in a 37° C. water bath, monitored closely and removed from the water bath once only a small bit of ice was remaining (2-3 min). Vial contents were transferred into the 50 mL centrifuge tube containing prepared MSC culture medium and the cell suspension was mixed well, then centrifuged at 350×g for 6 minutes. The supernatant was aspirated and the cells were resuspended in 60 mL of RoosterNourish-MSC-XF medium. Two 50 mL tubes were used and the 60 mL was split into two tubes. 10 mL of cells were seeded into each of the six vitronectin coated T-150 CellBind flasks. 20 mL of MSC culture medium was added to each flask, which was transferred into an incubator (37° C., 5% CO2) and the entire surfaces were covered evenly with medium.

The following day cell growth monitoring was begun. Cell confluency was monitored microscopically. Cells were ready to be harvested at >80% confluency. One liter of serum free medium (1 liter Dulbecco's Modified Eagle's medium (DMEM) with high glucose, I-glutamine, phenol red, and sodium pyruvate; Thermo Fisher Scientific, Waltham, MA) and 10 mL of stock Penicillin-Streptomycin (10,000 IU Pen and 10,000 IU Strep; Thomas Scientific, Swedesboro, NJ) was added.

Then 4 mL of sterile 2% weight/volume (w/v) molten agarose solution in phosphate buffered saline (PBS) (250 mL of agarose+PBS, 5 g of agarose) was added to large clean sterile molds designed to make 721 microtissues. A spatula was used to ensure the bubbles are not trapped in-between the posts. 1 liter of serum free medium was used to equilibrate gels (3 changes), then the molds were covered with large sterile glass slides (75×50 mm, Fisher Scientific, Hampton, NH) to flatten the agarose. After the agarose solidified (˜15 min), the gel was gently separated from the mold and transferred to a 6 well plate. This process was repeated until a total of 54 gels had been made (nine 6 well plates). 4 mL of Serum Free Medium (SFM) was added to each well (216 mL total) and the plates were transferred to a 37° C. incubator and incubated for ˜2 hours. The medium was aspirated off and 4 mL of SFM was added to each well (216 mL total). Plates were returned to the 37° C. incubator for overnight incubation.

The medium was aspirated off the 6 well plates with gels and 4 mL of SFM was added to each well (216 mL total). Plates were transferred to the 37° C. incubator and incubated for ˜2 hours. For MSC cell harvest, spent medium was removed and 6.67 mL TrypLE (Thermo Fisher Scientific, Waltham, MA) was added to each flask. TrypLE was distributed evenly to cover all the cells and vessels were placed in 37° C. (5% CO₂) incubator. Culture was checked every 5 minutes until cells were detached from surface. Flasks were gently tapped to dislodge remaining cells from surface. 6.67 mL of RoosterNourish™-MSC-XF media (Roosterbio, Frederick, MD) was added to each flask to stop the TrypLE activity. The cell suspension was transferred into two 50 ml centrifuge tubes (˜40 mL in each tube) and centrifuged at 350×g for 6 min. The supernatant was aspirated. Total cell number of six T-150 CellBind flasks was a total of about 50 M. 10 μL of cells were pipetted into a hemocytometer to count cells. Cells were resuspended in 50/50 medium (equal volumes of Serum Free Medium Plus [SFM+; 500 mL bottle of Dulbecco's Modified Eagle's medium (DMEM) with high glucose, I-glutamine, phenol red, and sodium pyruvate, 25.3 mg of L-proline powder per 500 ml bottle (final concentration of 50 μg/mL L-proline), 16.3 mg of 2-phospho-L-ascorbic acid trisodium salt powder per 500 mL bottle (final concentration of 0.1 mM), 5 mL of stock Penicillin-Streptomycin per 500 ml bottle] and Serum Free Medium Advanced [SFMA; 500 ml bottle of Advanced DMEM, 10 mL of 100× stock Glutamax per 500 mL bottle (final concentration is 4 mM), 5 mL of stock Penicillin-Streptomycin per 500 ml bottle] to achieve desired cell concentration for seeding gels.

Medium was aspirated off from around the gel in the six well plates and carefully removed from the seeding chamber without damaging the gel. MSC cells were resuspended in 60 mL 50:50 medium to a concentration of 721,000 cells/mL (target 1,000 cells/microtissue; total of at least 54 mL to seed 54 gels). Gels were seeded with 721,000 cells/gel or 1,000 cells/micro-well for 721 micro-wells. 1 mL of this cell suspension was added to the seeding chamber of each gel. The drops around the gel were carefully distributed to get uniform cell seeding. Typically seeding was drop wise around the edge of the gel and finishing in the center to ensure all parts of the gel have cells. Plates were allowed to sit in the hood for 10 minutes before carefully transferring them to a 37° C. CO2 incubator. After 20 minutes, the plates were carefully removed from the incubator and 4 mL of 50:50 medium was carefully added to the area around the gel. The 4 mL was carefully added slowly down the side of the well to not disturb the middle of the gel where the cells are still settling (216 mL total). Plates were returned to the 37° C. CO₂ incubator.

Next day, medium was aspirated off and 4 mL of 50:50 medium was added to each well (216 mL total needed). Plates were returned to the 37° C. incubator. Two days later, medium was aspirated off, and 4 mL of 50:50 medium was added to each well. Plates were returned to the 37° C. incubator (216 mL total needed). Two days later, medium was aspirated off, and 4 mL of 50:50 medium was added to each well. Plates were returned to the 37° C. incubator (216 mL total needed). Two days later, medium was aspirated off, and 4 mL of 50:50 medium was added to each well. Plates were returned to the 37° C. incubator (216 mL total needed).

Microtissues were harvested and decellularized as follows. Pyrex glass bowls were set up on ice with a pitch so that solutions will run down and pool at the bottom. A sterile silicone rubber shelf was placed at about the halfway mark of the bowl to hold the gels in place and prevent them from sliding down the bowl. The bowl was coated with a solution of 0.1 mg/mL BSA in saline for a minimum of 60 seconds and rinsed with saline five times. Medium was aspirated off from around each gel on a 6-well plate and carefully removed from the seeding chamber without damaging the gel or aspirating up the microtissues. Two gels were transferred to the shelf of each glass bowl using a spatula. Because the spheroids tend to stick to any plastic surfaces, Iobind 2 mL Eppendorf tubes, 15 mL or 50 mL Savillex tubes and Iobind P1000 pipette tips were used to have maximum product recovery. The inside of the tip of a P1000 Genesee Scientific pipette tip was coated with 0.1 mg/mL BSA in saline. This step was completed anytime pipette tips were changed.

Cold PBS-PI+Triton X-100 [289.5 mL cold phosphate buffered saline, 1.5 mL Triton X-100, final concentration 0.5%, 6 mL of a 1 M stock ammonium hydroxide (final concentration 20 mM ammonium hydroxide), 3 mL of 100× stock Halt Protease Inhibitor Cocktail added to buffer immediately before use] was added into the bowl. A P1000 with a Iobind tip was used to dislodge the microtissues from each gel by squirting it the with cold PBS-PI+Triton X-100. Approximately six or more squirts distributed evenly over the gel dislodged all the microtissues. Gels were checked under the microscope to be certain all microtissues have been removed from the gel. The bottom of the glass bowl was rinsed with some of the PBS-PI+Triton X-100 to ensure that nearly all microtissues have been gathered. The microtissues were transferred into 2 mL Iobind Eppendorf tubes. Once all microtissues have settled in the tubes, the PBS-PI+Triton X-100 was aspirated off. All microtissues were pooled into five 2 mL tubes on ice. A Nageotte Counting Chamber was used to count spheroids (see Protocol: Counting Spheroids and ECM particles).

Tubes were placed in a micro-centrifuge and spun at 2,200 RPM for six minutes. PBS-PI+Triton X-100 was aspirated off and the microtissues were resuspended in 2 mL per each 2 mL tube of PBS-PI+Triton X-100. Tubes were incubated at 37° C. for 30 minutes. Tubes were placed in a micro-centrifuge and spun at 2,200 RPM for six minutes to pellet the microtissues. PBS-PI+Triton X-100 was aspirated off and the microtissues were resuspended in 2 mL per each 2 mL tube of PBS-PI+Triton X-100. Tubes were incubated at 37° C. for 30 minutes. Tubes were placed in a micro-centrifuge and spun at 4,000 RPM for six minutes to pellet the microtissues. PBS-PI+Triton X-100 was aspirated off and the microtissues were resuspended in 2 mL per each 2 mL tube of PBS-PI+Triton X-100. Tubes were placed in a micro-centrifuge and spin at 4,000 RPM for six minutes to pellet the microtissues. PBS-PI+Triton X-100 was aspirated off and the microtissues were resuspended in a 10 ml of PBS-PI (39.6 mL cold phosphate buffered saline, 0.4 mL of 100× stock Halt Protease Inhibitor Cocktail). Tubes were placed in a micro-centrifuge and spun at 4,000 RPM for six minutes to pellet the microtissues. PBS-PI was aspirated off and the microtissues were resuspended in 5 mL of PBS-PI and incubated at 37° C. for 30 minutes. Tubes were placed in a micro-centrifuge and spun at 4,000 RPM for six minutes to pellet the microtissues. PBS-PI was aspirated off and the microtissues were resuspended in 10 mL of DNA/RNA digestion solution (1 mL of 10× stock DNase buffer, 8.7 mL Milli Q water 200 μL of 10,000 U/mL stock DNase I, RNase-free (final concentration 200 U/mL), 2 μL of 100 mg/mL (7000 units/mL) stock RNase A (final concentration 20 g/mL), 100 μL of 100× stock Halt Protease Inhibitor Cocktail added just before use, filter sterilized]. All tubes were secured and incubated at 37° C. for 72 hours. Every morning the tubes were turned upside down to allow the microtissues to disperse all throughout the tube.

ECM particles were washed and stored as follows. Tubes were placed in a micro-centrifuge and spun at 2,200 RPM for six minutes to pellet the ECM particles. The DNA/RNA digestion solution was aspirated off and the ECM particles were resuspended in 5 mL of saline suitable for injection. The tubes were incubated at 37° C. for 30 minutes. The ECM particles were allowed to settle naturally and then the saline was aspirated off and the microtissues were resuspended in 10 mL of saline total. The tubes were incubated at 37° C. for 30 minutes. The ECM particles were allowed to settle naturally and then the saline was aspirated off and the microtissues were resuspended in 10 mL of saline total. The ECM particles were allowed to settle naturally and then the saline was aspirated off and the microtissues were resuspended in 10 mL of saline total. ECM particles were stored in saline at 4° C. A Nageotte Counting Chamber was used to count ECM particles (See Protocol: Counting Spheroids and ECM particles).

Histology of Decellularized MSC ECM Particles: Decellularized MSC ECM particles were fixed in 10% buffered formalin (Fisher 427098) paraffin-embedded, sectioned at 5 μm then stained with hematoxylin and eosin (H&E) or SIRIUS RED™ (Polyscience, Warrington, PA, 24901-250) to examine the presence of cell nuclei or fibrillar collagen deposition of decellularized MSC ECM particles, respectively.

dsDNA Concentration of Decellularized MSC ECM Particles: Double stranded (dsDNA) concentration of decellularized MSC ECM particles were measured as previously described by Blaheta et al. (1998)ⁱ. Decellularized microtissue ECM particles that were collected into a tube were digested in papain solution (MilliporeSigma, P4762, 125 μg/mL) in a sonicator for 72 hours at 65° C. The dsDNA concentration of the digested ECM was measured using QUANT-IT™ PICOGREEN™ dsDNA Assay Kit (Thermo Fisher Scientific, P7589) per manufacture's protocol.

Collagen Content of Decellularized MSC ECM Particles: Collagen content of MSC ECM particles was measured as previously described by Cissell et al. (2017).ⁱⁱ MSC ECM particles were fixed in 10% formalin and stored at 4° C. until further processed. Fixed MSC ECM particles were collected into a tube and washed three times with 1×PBS, then digested in papain solution (MilliporeSigma, P4762, 125 μg/mL) in a sonicator for 10 days at 65° C. The digested MSC ECM particles were measured using a modified hydroxyproline assay, as described by Cissell et al. (2017).

Sulphated Glycosaminoglycans (sGAG) Content of MSC ECM Particles: sGAG content of MSC ECM particles was measured using the 1,9-dimethylmethylene blue (DMMB) assay as described by Farndale et al. (1982)ⁱⁱⁱ and Whitley et al. (1989).^{iv} Microtissues were fixed in 10% formalin and stored at 4° C. until further processed. Fixed microtissues were collected into a tube and washed three times with 1×PBS, then digested in papain solution (MilliporeSigma, P4762, 125 μg/mL) in a sonicator for 10 days at 65° C. The digested microtissues was measured using the DMMB assay as described by Farndale et al. (1982) and Whitley et al. (1989).

Proteomics of Decellularized MSC ECM Particles: Decellularized MSC ECM particles that were collected into a tube underwent proteomics procedures as previously described by Naba et al. (2015)^{v} for proteomics analysis of the microtissues. Mass spectrometry by data dependent acquisition (DDA) and data analysis with Proteome Discoverer 2.3 (1% FDR) were used for the proteomics analysis of ECM proteins. An established iBAQ algorithm as described by Schwanhäusser et al. (2011)^{vi} was used to semi-quantity ECM components (by % molar of total ECM proteins) by dividing each individual protein's total intensity with the theoretical number of tryptic peptides between 6 and 30 amino acids in length (PeptideMass, SIB Swiss Institute of Bioinformatics).

Mechanical Stiffness of MSC ECM particles: Samples tested were plated on collagen-coated coverslips and incubated on the coverslips at 4° C. for 48 hrs prior to testing. Force measurements were collected using an atomic force microscope (AFM, MFP-3D-BIO, Asylum Research, Santa Barbara, CA) connected to a Nikon Eclipse Ti—U epifluorescence microscope (Nikon, Chicago, IL). The cantilever used had a spring constant of 0.03 N/m. Multiple testing sessions were conducted for the various samples to account for systematic errors. Force versus indentation data were analyzed using custom MATLAB scripts (The MathWorks, Natick, MA) utilizing the Hertz contact model.

All experiments were carried out at room temperature in fluid environments. The AFM was allowed to equilibrate before tests to minimize deflection laser and/or piezo drift. Force maps were collected for a variety of samples using a force mapping technique in contact mode. In brief, individual force curves were taken at discrete points across a region of interest. During analysis, the spatial arrangement of the data was retained to create a matrix of elastic modulus values. Force indentation data were sampled at 5 kHz with an approach velocity of 10 μm/sec. A trigger force of about 4 nN was used for all samples with the deflection set to 100 nM. Scan size used was 5 μm and the resolution was 4×4 pts.

### Example 13. In Vitro Biocompatibility of ECM Particles

Decellularized adult or fetal heart ECM particles were seeded with HCM or HCMEC and incubated for 24 hours. To examine proliferation, the nucleoside analog EdU (5-ethynyl-2′-deoxyuridine) was added 24 hours after cell seeding. Cells on ECM particles in EdU were cultured for another 24 hours (HCMEC) or 48 hours (HCM), then fixed in 10% formalin for immunohistochemical evaluation using the Click-iT™ EdU Cell Proliferation Kit (Thermo Fisher Scientific) according to the manufacturer's protocol.

### Results; Principal Component Analysis (PCA) of MSC ECM

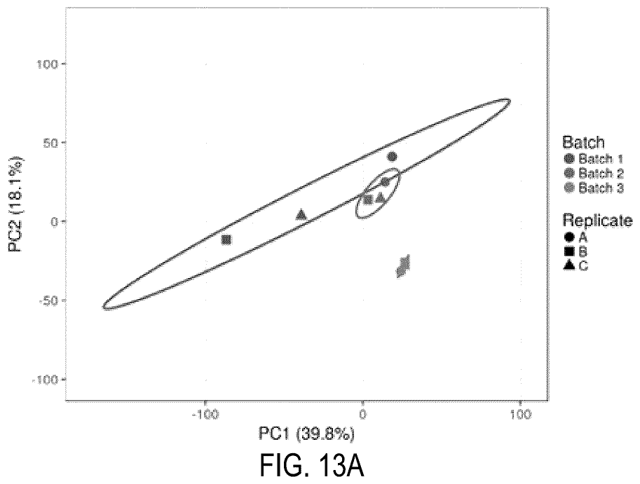

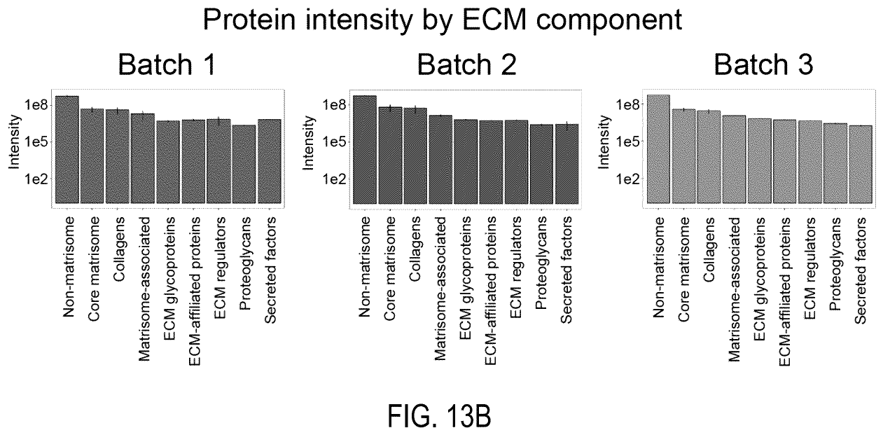

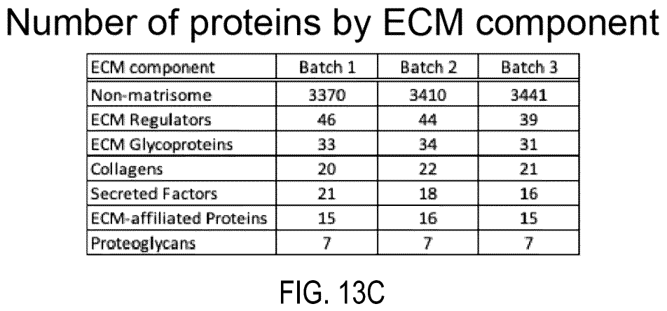

FIGS. 13A-13C provides an overview of MSC ECM particle proteomics data, generated from three particle batches in technical triplicates, showing protein intensity variability among samples, protein intensity by matrisome category, and number of proteins identified by matrisome category. FIG. 13A is a principal component analysis (PCA) plot showing protein intensity variability among MSC ECM particle batches and technical replicates analyzed by mass spectrometry. Sample variability appeared to progressively decrease with each batch preparation. It should be noted that, while a higher number of non-matrisome proteins were identified compared to matrisome proteins, the relative protein intensity of matrisome vs. non-matrisome components among batches indicates that MSC ECM particles are highly enriched in matrisome proteins. FIG. 13B shows MSC ECM particle matrisome composition, by average protein intensity, of each batch analyzed by mass spectrometry. Error bars represent standard deviation among technical triplicates. FIG. 13C shows MSC ECM particle matrisome composition, by number of unique proteins, of each batch analyzed by mass spectrometry.

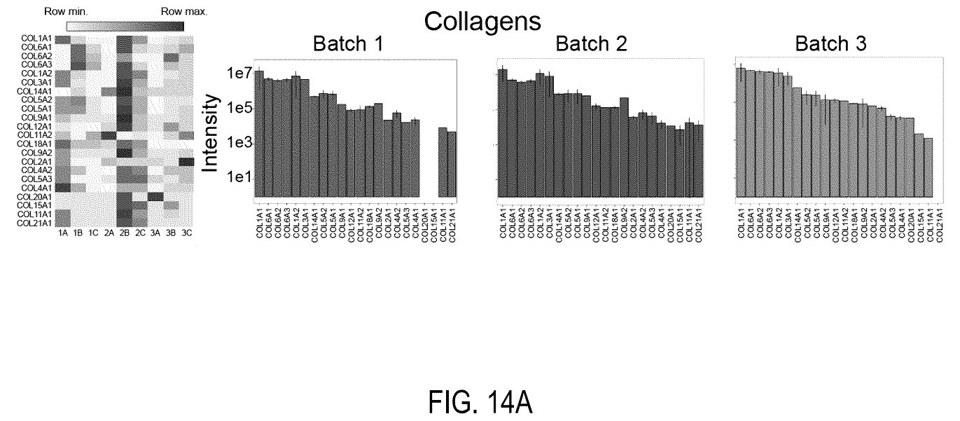

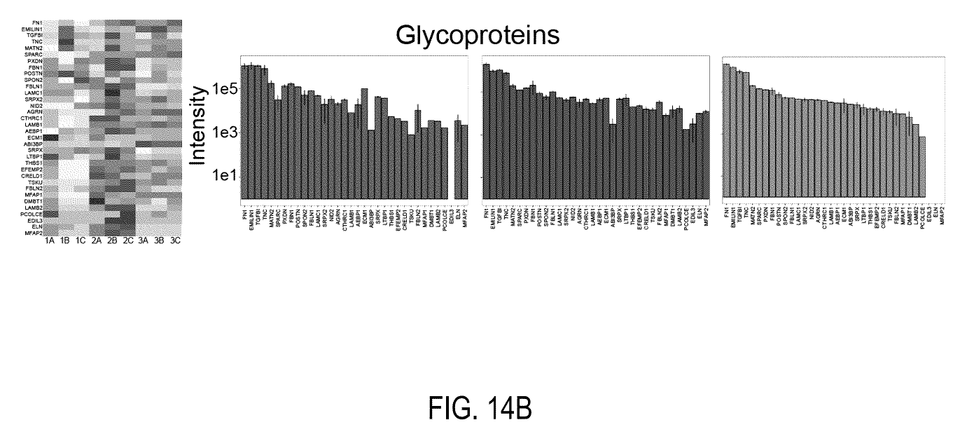

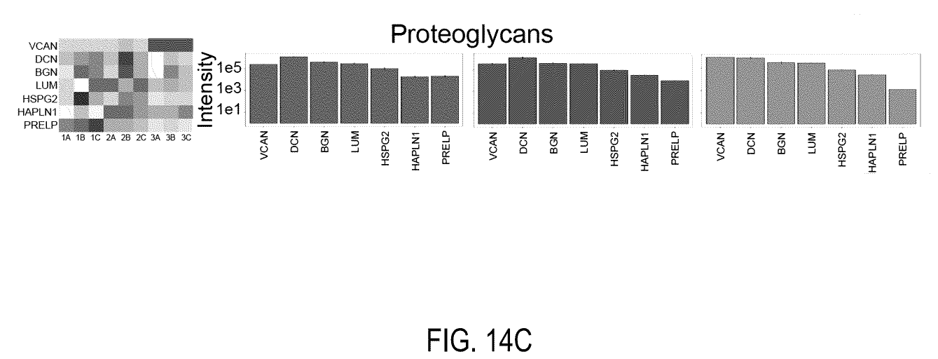

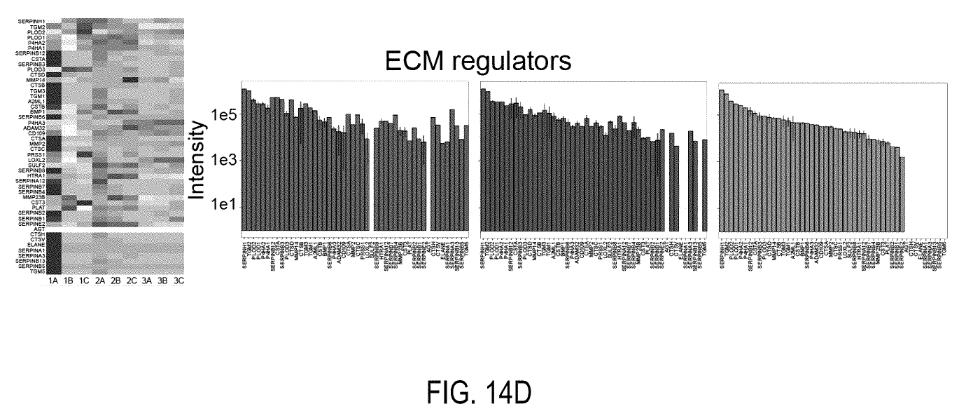

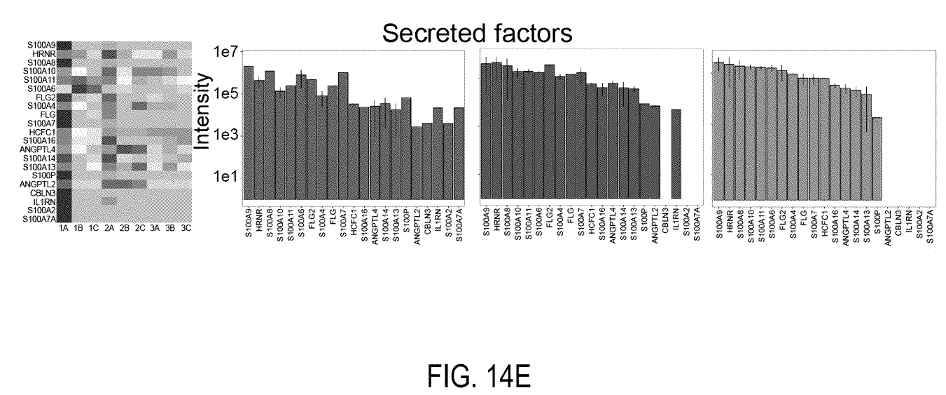

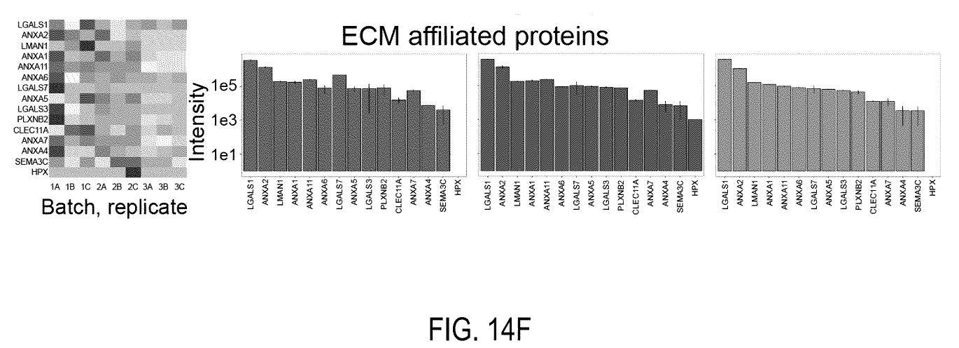

FIGS. 14A-14F show intensity of the following ECM components: collagens, glycoproteins, proteoglycans, ECM regulators, secreted factors and ECM affiliated proteins. The left side provides heatmaps showing MSC ECM particle batch and technical replicate protein intensities of matrisome category proteins analyzed by mass spectrometry. Darker coloration indicates higher protein intensity relative to the row. The right side shows MSC ECM particle matrisome category component average protein intensities measured in each batch analyzed by mass spectrometry. Error bars represent standard deviation among technical triplicates. This figure shows that major ECM components are represented at similar levels across batches.

Analysis of MSC ECM Matrisome: The ECM is a dynamic and complex molecular network with distinctive biochemical and structural characteristics.^{vii} In analyzing the structure and functions of extracellular matrices, one would like to have a complete “parts list”—a list of all the proteins in any given matrix and a larger list of all the proteins that can contribute to matrices in different situations (the “matrisome”). The matrisome consists of a group of proteins encoded by genes for core ECM proteins (collagens, proteoglycans, and ECM glycoproteins) and ECM-associated proteins (proteins structurally resembling ECM proteins, ECM remodeling enzymes, and secreted factors).^{viii}

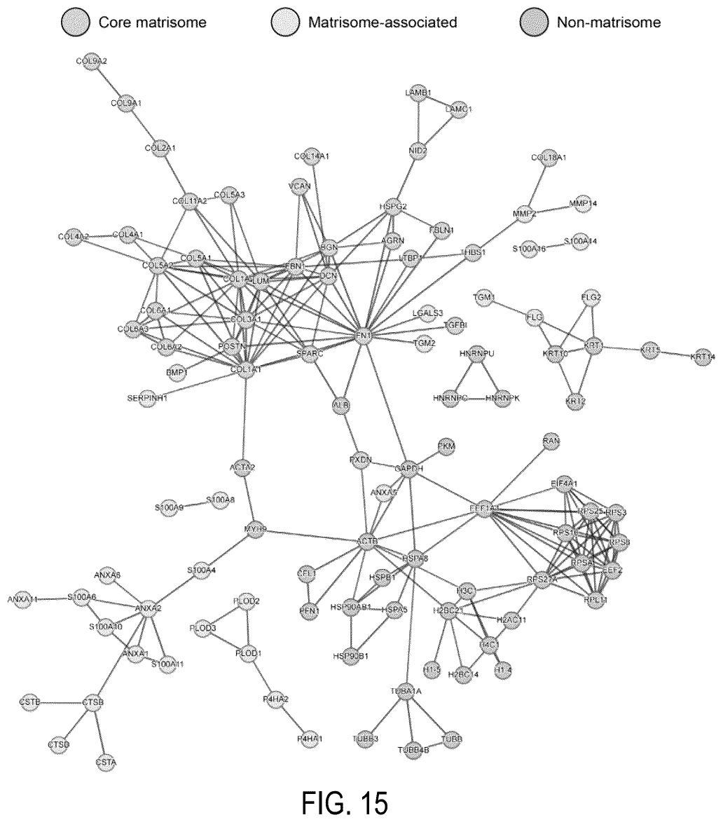

The present study analyzed the matrisome of MSC ECM. FIG. 15 shows the top 50 core matrisome, matrisome-associated, and non-matrisome proteins in MSC ECM particles with stringDB interaction map, highest confidence interactions (StringDB interaction score ≥0.9) and disconnected nodes removed. Top disease-gene associations included, but are not limited to, Ehlers-Danlos syndrome, aortic aneurysm, osteogenic imperfecta, amyloidosis, corneal disease. Top tissue expression included, but are not limited to, bronchial epithelium, bone, aorta endothelium, ear, and corneocyte. Top human phenotypes included, but are not limited to, Ehlers-Danlos syndrome (cigarette-paper scars), arterial rupture, bladder diverticulum, hyperflexibility of wrist, and atrophic scars.

Comparison of MSC ECM Matrisome with Human ECM Microtissues: In WO 2022/140530,^{ix} we disclosed and characterized decellularized mammalian ECM particles or morsels from cultured human cells such as heart or lung cells. In the present study, these human ECM microtissues were compared with the MSC ECM particles of the present disclosure.

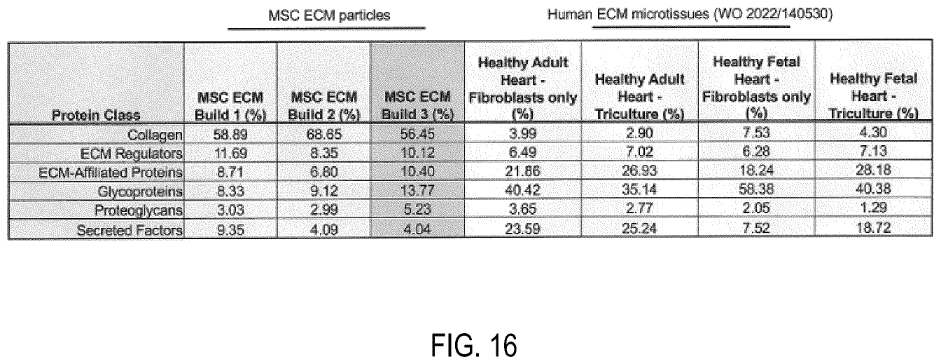

The table in FIG. 16 provides proteomic matrisome analyses showing that the matrisome composition of MSC ECM particles of the present disclosure differ from the human ECM microtissues described in WO 2022/140530. Specifically, MSC ECM particles have substantially higher collagen content by percentage ECM composition, lower ECM-affiliated protein percentage, lower glycoprotein percentage, and lower secreted factors percentage. These data suggest that MSC ECM particles are more representative of a core matrisome composition and are thus more broadly applicable and appropriate for ECM delivery and treatment.

Analysis of TGFβ Pathway Regulators: Regulation of production and turnover of ECM components is essential for tissue homeostasis and function. TGFβ exerts its effects on cell proliferation, differentiation, and migration in part through its capacity to modulate the deposition of ECM components.^{x} TGFβ is secreted as part of a tripartite complex from which it must be released in order to bind to its receptor. Sequestration of latent TGF-β in the extracellular matrix (ECM) is crucial for proper mobilization of the latent cytokine and its activation.^{xi}

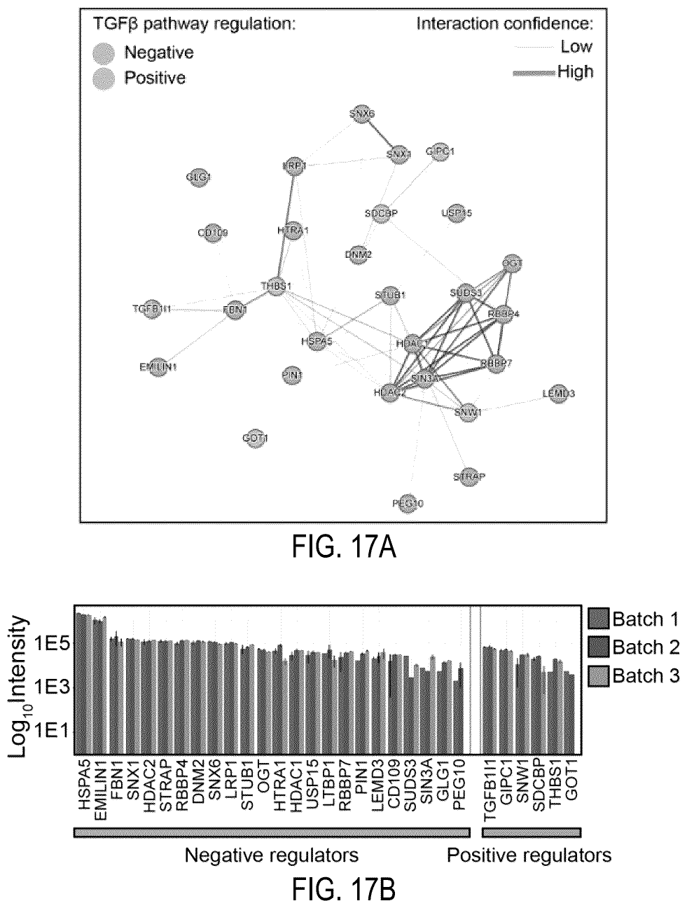

The present study provides an analysis of TGFβ pathway regulators. FIG. 17A shows the interaction network of TGFβ signaling pathway regulators in ECM particles. Edges represent interaction confidence, with thicker edges representing higher confidence and thinner edges representing lower confidence. FIG. 17B show intensities of TGFβ pathway regulators in ECM particle batch proteomics. This figure shows that MSC ECM particle proteins form protein-protein interaction networks, both with matrisome and non-matrisome proteins, which uphold their biological function.

### Example 14 Use of MSC ECM Particles in the Treatment of Myocardial Ischemia

An important purpose of this study was to investigate the potential therapeutic effects of ECM particles produced from decellularized 3D microtissues of human mesenchymal stem cells in a mouse model of myocardial ischemia.

Production of ECM Particles from Spheroids: To produce laboratory-grown human 3-D ECM particles, human MSCs (Xeno-Free Human umbilical cord-derived mesenchymal stem/stromal cells; Rooster-Bio) were cultured and expanded in number in the MSC-XF medium (Rooster-Bio) on T-225 Cell Bind flasks (Corning) coated for 1 h with a vitronectin solution (1.5 μg/mL) (Thermo Fisher) in a phosphate buffered saline (PBS) solution. A molten solution of 2% sterile ultrapure agarose (Fisher Bioreagents) in PBS was poured into our custom silicone rubber molds, each with 721 small posts. When the agarose gelled, it created a negative replica of the posts, thus forming 721 microwells of agarose. The gels were carefully removed from the molds and transferred to 6-well plates (Corning) and equilibrated three times with serum-free Dulbecco's modified Eagles medium (DMEM) (Thermo Fisher) with penicillin and streptomycin. For spheroid culture, we used a customized 50:50 cell culture medium with added ascorbic acid (1.25 mg/mL) and proline (25.3 mg/mL), following a protocol we have previously optimized for ECM production. To form spheroids, the flasks containing MSCs were washed with PBS and treated with a trypsin solution (TrypLE; Life Technologies). Trypsinized cells were spun down (350 g for 6 min) and resuspended in 50:50 medium, counted, and seeded onto the micro-molded agarose gels at a density of 7.2×10⁵ cells per gel resulting in about 1,000 cells per spheroid. After the cells had settled (30 min), the gels were returned to the incubator and cultured for 7-10 days at 37° C., 10% CO₂. The resulting spheroids were harvested by flushing the surface of each gel with cold PBS 1× Halt Protease Inhibitor cocktail (PI) (Thermo Fisher)+20 mM ammonium hydroxide, and 0.5% Triton X100 (Millipore Sigma) (PBS-PI+Triton X100).

The spheroids were pooled, pelleted by centrifugation (2,200 rpm for 6 min in a micro-centrifuge), and washed three times for 30 min with cold PBS-PI+Triton X100 followed by three 30-min washes in PBS-PI at 37° C. After each wash, particles were collected by centrifugation (4,000 rpm for 6 min in a microcentrifuge). The particles were next incubated at 37° C. for 72 h in the DNA/RNA digestion solution [DNase I, RNase-free 200 U/mL (Sigma-Aldrich), RNase A 20 μg/mL (Qiagen)] with PI. The particles were washed several times with saline suitable for injection (Quality Biological), resuspended in saline, and stored at 4° C. ECM particles were stored at 4° C. for up to 2 wks before testing in the MI model and 4-6 wk before the biodistribution experiments. ECM particles were counted using a Nageotte Counting Chamber.

Protein concentration was determined using the bicinchoninic acid (BCA) assay. DNA content was determined using the Picogreen assay. An acceptable level of nucleic acid was 95%. Preparation for Injection of ECM Particles ECM particles measuring 200 microns in diameter were suspended in a sterile saline solution at a concentration of 63,400 ECM particles per mL. Before loading with ECM particles or saline control, a 10-uL syringe (Hamilton, 7642-01) was precoated with human serum albumin (HSA) to prevent sticking of ECM particles. A total of 50 units (500 μL) of sterile filtered 0.1 mg/mL HSA in PBS was drawn up into the syringe. After 60 s, the HSA solution was dispensed, and the syringe was washed five times with sterile saline and the syringe loaded with 10 μL of ECM or sterile saline solution.

Animals: Eight to ten-wk-old male and female FVB mice (Charles River Laboratories, Strain No. 207) were used in this study. All animals were housed in the Coro West Building Barrier Facility at Rhode Island Hospital and underwent acclimatization before echocardiogram and surgery. Sample size, using power analysis and experience from the previous studies [see Teixeira et al.], was determined n=8 animals per group. However, to account for perioperative mortality, we used 10 mice per group. Mice were allocated to one of the two groups, with intramyocardial delivery of 630 ECM particles suspended in 10 uL (n=10 animals/group) or 10 uL saline control (n=10 animals/group) by intramyocardial injection immediately after the ligation procedure, confirmed by tissue blanching. All experimental procedures were approved and carried out in accordance with the Institutional Animal Care and Use Committee (IACUC) of Rhode Island Hospital (CMT No. 5017-22).

Echocardiography: Preoperatively and again at postoperative days (POD) 3, 7, 14, 21, and 28, mice underwent echocardiographic assessment as previously described. Mice were anesthetized using 2% continuous inhalant isoflurane anaesthesia. Active temperature monitoring to maintain a normothermic core temperature and heart rate monitoring to maintain a heart rate between 400 and 500 beats/min were used. Left ventricular function was evaluated via two-dimensional (2-D) echocardiography (Vevo 2100; FUJIFILM Visual Sonic Inc.) and ejection fraction was calculated using Simpson's method. Shapiro-Wilk test was used to determine data normality. Of note, one mouse from the ECM cohort died on POD 28 before final echo. Prior timepoints were analysed both with and without inclusion of this mouse. Differences remained statistically significant or not regardless of inclusion, so to reduce potential type II error, the authors decided to include this data point.

Left Anterior Descending Coronary Artery Ligation and Injection of ECM Particles: As previously described, mice were anesthetized using 3% inhaled isoflurane anaesthesia and intraperitoneal ketamine injection (100 mg/kg) to facilitate endotracheal intubation and positive pressure ventilation (Mini Vent Type 845; Harvard Apparatus). For analgesia, a subcutaneous dorsal fat pad injection of Buprenorphine ER (1 mg/kg) was used. After successful intubation, confirmed with observed bilateral chest rise, inhaled isoflurane was reduced to 2% for anaesthesia maintenance. A depilatory agent was applied to the left chest wall to remove fur, and using betadine swabs, a sterile surgical field was prepped. A left lateral thoracotomy was then performed exposing the heart at which point the pericardium was bluntly stripped away and left anterior descending coronary artery (LAD) identified. At a point 2 mm inferior to the left atrial appendage, an 8-0 nylon suture was used to ligate the LAD completely (permanent ligation, without reperfusion) as described in our recent publication. Confirmation of cessation of blood flow, that is, myocardial infarct (MI), through the LAD was confirmed via observed distal blanching.

Mice were then allocated to one of the two groups, 630 ECM particles suspended in 10 μL (n=10) or 10 μL saline control (n=10) intramyocardial (I/M) injection. I/M injections were given perioperatively to two periinfarct locations distal to the completely ligated LAD as determined by area of blanching. Two periinfarct locations of each heart received 10 UL of either ECM or saline in the myocardium of each periinfarct location immediately after the MI was established. Following intramyocardial injection, the thoracotomy was closed in two layers. First, a 6-0 vicryl stitch was used to reapproximate the deep layer of the chest wall. Subsequently, the skin and subcutaneous tissue were reapproximated with interrupted 6-0 vicryl stitches. Mice were then observed during wean from anaesthesia and extubated.

Biodistribution of Injected ECM Particles: ECM particles were conjugated to the fluorophore, IVISense Tomato Lectin 680 (Revvity, Waltham, Massachusetts), at a concentration of 1:100. After the addition of the fluorophore, the solution was allowed to incubate for 2 h at room temperature after which the solution was centrifuged and supernatant removed. Repeated PBS washes were performed for an additional three times in 30-min intervals. After the third wash, ECM particles were resuspended in PBS and stored at 4° C. until injection.

In a previously described protocol by our laboratory, mice were allocated into groups: 1) 10 μL ECM particle intramyocardial injection (n=3), 2) 10 μL saline+TL680 intramyocardial injection (n=5), 3) 10 μL ECM+TL680 intramyocardial injection (n=4), 4) 100 μL ECM tail vein injection (n=3), 5) 100 μL saline+TL680 (n=5), and 6) 100 μL ECM+TL680 (n=4). Intramyocardial and tail vein injections were administered immediately following LAD ligation. Two hours post injection, mice were euthanized via CO2 inhalation and cardiac, pulmonary, hepatic, splenic, and renal tissue harvested. End organ epifluorescence was measured using the In vivo Imaging System (IVIS) (Revvity, Waltham, MA) and analysed with Living Image software (PerkinElmer, Waltham, MA).

Immunohistochemistry: In a process previously described by our laboratory, frozen tissue sections at 5-microns thick were prepared and mounted on slides. Masson's Trichrome, terminal deoxynucleotidal transferase-mediated biotin-deoxyuridine triphosphate nick end labelling (TUNEL), Ki67, and anti-3-nitrotyosine staining were performed by iHisto (Salem, MA). Images were obtained using the Motic EASYSCAN Infinity bright-field slide scanner (Motic Kowloon, Hong Kong) at ×20 magnification. QuPath software was used to quantify percent positive trichrome staining and percent left ventricle infarct size. QuPath automated detection program was used to determine percent abundance of TUNEL, Ki67, and anti-3-nitrotyosine stains.

Immunofluorescence: Similar to immunohistochemistry, frozen tissue section at 5-microns thick was prepared and mounted on slides. Transforming growth factor beta (TGFb), tumour necrosis factor alpha (TNFa), Interleukin-6 (IL-6), cluster of Differentiation 86 (CD86), and cluster of Differentiation 163 (CD163) immunofluorescent staining and Isolectin B-4 and alpha-smooth muscle actin (a-SMA) duplex immunofluorescent staining were performed by iHisto (Salem, MA). Images were obtained using Pannoramic MIDI II slide scanner (3-D Histech, Hungary) at ×20 magnification. Image analysis was performed using QuPath software. Arteriolar count was determined using thresholding to define positive aSMA and a size cut-off of 100 um object diameter per area of tissue section.

Proteomic Analyses: ECM particle proteome was assessed in triplicate aliquots containing 100-Ig particles each from three separate batches (Omix Technologies, Colorado). Briefly, particles were solubilized and reduced in 10% SDS, 100 mM TEAB pH 8.5, 5 mM TCEP. Samples were alkylated, acidified, and transferred to an S-trap microcolumn for desalting and trypsinization. LCMS/MS was performed on a Bruker TimsTOF SCP LC-MS/MS in data-independent acquisition (DIA) mode with considerations for ECM detection including hydroxylation of proline and lysine residues.

To assess the effects of ECM particles on post-MI heart tissue proteome (POD 28, n=3), 30 mg tissue sections were excised from ischemic and contralateral perfused heart regions (i.e., proximal left ventricular region unaffected by the infarct), snap-frozen, and then lyophilized. Powdered samples were fractionated into “cellular,” “soluble ECM”, (SECM), and “insoluble ECM” (iECM) fractions using a series of sequentially harsher buffer conditions (Omix Technologies, Colorado).

Briefly, cellular fractions were generated from supernatants after incubation with HS buffer (0.25% CHAPS, 3M NaCl, 50 mM Tris-HCl PH 7.4, 25 mM EDTA, 1× protease salt). sECM fractions were generated from supernatants after incubation with buffer comprising 6 M guanidinium hydrochloride (Gnd-HCl) and 100 mM ammonium bicarbonate (ABC), pH 9. Finally, iECM fractions were generated by incubating the remaining pellet with a buffer comprising 1 M NH₂OH, <6MGnd-HCl, and 100 mM ammonium bicarbonate (ABC), pH 9. Finally, iECM fractions were generated by incubating the remaining pellet with a buffer comprising 1 M NH₂OH, <6MGnd-HCl, ABC, 4% NaOH, and 0.02 K₂CO₃, pH 9.0-9.2. Fractions were digested using a standard filter-aided sample preparation (FASP) protocol involving mixing with 8 M urea, reduction in 10 mM TCEP, and alkylation with CIAA before trypsinization. Cleaned peptides were analyzed using a Fusion Lumos LC-MS/MS in DIA mode, with considerations for ECM including hydroxylation of proline and lysine.

Statistical Analysis: Data from echocardiography, histology, and biodistribution results underwent statistical analyses as performed via unpaired t test, Mann-Whitney U test, or ANOVA to determine significance as appropriate. Proteomic data of ECM particles were assessed based on average log-transformed intensity and relative standard deviation (RSD) among batch replicates (n=3). Within batches, proteins with 20% RSD were considered to be detected with confidence. Comparisons among batches were performed using Student's t test (P value 0.05). Matrisome components were assigned using Matrisome AnalyzeR. Mouse heart proteomics comparisons between ECM and saline-injected groups (n=3) were performed using Student's t test of log-transformed protein intensities per fraction and heart region (P value 0.05). Fold-changes were calculated based on average intensities per fraction and heart region. Proteomics data were assessed and interpreted using bioinformatic tools Matrisome AnalyzeR, Gene Ontology Biological Process (P value 0.05), and StringDB (interaction score 0.7).

Biodistribution of ECM Particles when Administered Intramyocardially versus Intravenous Route: To determine an effective mode of delivery of ECM particles to the ischemic heart, we injected Tomato Lectin 680 fluorophore labelled ECM particles either directly into the is chemic myocardium or by tail vein injection. When delivered intramyocardially (I/M) in the ischemic myocardium, epifluorescence was significantly increased in IM NS b TL680 and IMECM+TL680 compared with non-labeled IM ECM particles (P<0.05 for all). There was a statistically significant, but clinically inconsequential increase in epifluorescence in the IM NS+TL680 group when compared with the IM ECM particles+TL680 cohort (P=0.0165). There was a significant increase in cardiac epifluorescence in the IM ECM particles+TL680 groups compared with all peripheral tail vein (TV) injections (P<0.05 for all) (not shown). No significant uptake was observed in peripheral organs of any group, when ECM particles were administered either by IM or TV (not shown). Together, these data suggested that IM injection was an effective mode of delivery of ECM particles to the ischemic myocardium and particles remained localized in the heart. However, intravenous (TV) administration was not an effective mode of delivery.

Preoperative Cardiac Function Analysis by Echocardiography: Preoperative baseline left ventricular function was determined by echocardiography for all mice. No significant difference was observed between the control mice and mice injected intramyocardial with ECM particles in ejection fraction (EF) or fractional shortening (FS) (P=0.90 and P=0.85, respectively). Mean EF for saline control group was 61.84%, and mean EF for ECM particles group was 62.30%. Mean FS for saline control group was 36.56%, and mean FS for the ECM particles group was 37.14% preoperatively (not shown).

Post-MI Changes in Cardiac Function by Injection of ECM Particles: Postoperative left ventricular function was determined by echocardiography for all mice. A significant difference in fractional shortening (FS) was observed across multiple time points. At POD 7, there was a trend toward improved FS in the ECM particles group when compared with controls 23.01% versus 16.85% (P=0.05). A significant improvement in FS was observed in the ECM particles group at POD 14, POD 21, and POD 28 when compared with controls (P=0.04, P<0.001, and P=0.02, respectively) (not shown).

There was an initial trend of improved mean EF in the ECM particles group when compared with controls; however, this did not reach statistical significance during the study (P=0.09, P=0.07, P=0.1 at POD 7, POD 14, and POD 21, respectively).

Reduction in Infarct Size by Injection of ECM Particles: Masson's Trichrome staining revealed a significant difference in left ventricle (LV) infarct size between the ECM particles and control groups. Mice injected with ECM particles had an average LV infarct area of 11.62%, whereas the control average infarct area was 17.51% (P=0.04), suggesting an 33% reduction in infarct size by injection of ECM particles (not shown).

Increase in Vascular Density in the Ischemic Myocardium by Injection of ECM Particles: Immunofluorescence using Isolectin B4 for endothelial cells demonstrated a significant increase in capillary density in the ischemic area of hearts injected with ECM particles when compared with controls (P=0.01). No significant differences between groups were observed in the remote area or border zone (P=0.09 and P=0.37, respectively). Immunofluorescence using aSMA for vascular smooth muscle cells showed no significant differences in arteriolar density between ECM particle injected and control hearts in remote areas and infarct border zone (P=0.40 and P=0.62, respectively). However, there was a strong trend toward increased arteriolar density in the ECM particle injected hearts in the ischemic area compared with control (P=0.12). These data corroborate post-MI improvement by ECM particles in FS and infarct size.

Cellular Proliferation: Ki67 staining showed no significant difference in cellular proliferation in the heart tissue between mice injected with ECM particles and controls at the remote area (perfused area), infarct border zone, or ischemic area (P>0.05 for all) (not shown).

Apoptosis: TUNEL staining showed no significant difference in cellular apoptosis in the heart sections between mice injected with ECM particles and controls at the remote area, infarct border zone, or ischemic area (P>0.05 for all) (not shown).

Oxidative Stress: Anti-3-nitrotyosine immunohistochemical staining showed no significant difference in oxidative stress between mice injected with ECM particles and controls at the remote area, infarct border zone, or ischemic area (P>0.05 for all) (not shown).

Acute Inflammatory Markers: Immunofluorescence staining showed no significant difference in TNFa or IL-6 level between mice injected with ECM particles and controls at the remote area, infarct border zone, or ischemic area (P>0.05 for all) (not shown).

These findings suggest that ECM particles do not induce a major acute inflammatory response.

M1 and M2 Macrophage Markers: Immunofluorescence staining for the M1 macrophage marker, CD86, showed no significant difference between mice injected with ECM particles and controls at the remote area, infarct border zone, or ischemic area (P>0.05 for all). Immunofluorescence staining for the M2 macrophage marker, CD163, was significantly increased in the remote area of mice injected with ECM particles when compared with controls (P=0.006). However, there were no significant differences observed in CD163 staining in the border zone or ischemic area (P>0.05 for both) (not shown). Together, these findings suggest that there were no significant differences in immune responses between ECM-treated and nontreated ischemic myocardium as well as in infarct border zones as evidenced by lack of difference in M1 and M2 macrophage markers in these regions.

TGFb: Immunofluorescence staining for the growth factor TGFb showed no significant difference between mice injected with ECM particles and controls at the remote area, infarct border zone, or ischemic area (P>0.05 for all) (not shown).

Proteomics: A total of 1,504 total proteins were identified by fractionation and LC-MS/MS of mouse hearts. Core matrisome protein intensity was significantly higher in sequential fractions (i.e., iECM>sECM>Cellular). Consistently, non-matrisome protein intensity was significantly lower in iECM fractions. In ischemic regions, 36 proteins were significantly upregulated and 22 were downregulated in ECM-treated hearts. In perfused regions, 80 proteins were significantly upregulated and 58 were downregulated (not shown). Shared protein detections were more frequent by solubility fraction. sECM fractions had the highest number of detections, with over 1,000 detected proteins, followed by cellular fractions with around 750 proteins detected, and finally iECM fractions with about 300 proteins detected (“Group size” is not shown). This is reflected in shared detections across analytical groups, where sECM fractions had the highest number of common protein detections exclusively in sECM fractions, followed by cellular fractions with 264 exclusive common detections. Two hundred ten proteins were commonly detected in both sECM and cellular groups. Two hundred nine proteins were commonly detected across all groups. Interestingly, 95 proteins were commonly detected in iECM and sECM fractions but not in cellular fractions, and only 7 proteins were commonly detected in just iECM fractions (“Intersection size” is not shown), linked to lower number of proteins detected in iECM fractions.

Principal component analyses (PCA) of log-transformed protein intensities showed more defined separation by treatment in tissue sections and solubility fractions with a higher number of significantly differential proteins (not shown). Consistently, ischemic sections showed less separation by PCA than perfused sections (not shown). StringDB functional interaction analysis of significantly differential proteins in ischemic regions indicated interconnecting clusters representing muscle contraction and fatty acid b-oxidation. Proteins in these clusters were upregulated in ECM particle-versus saline-injected mice. Furthermore, these clusters were shared with most abundant ECM particle proteins, which also indicated interconnecting clusters representing ECM organization and actin organization. As expected, ECM particle proteins representing ECM organization cluster were highly abundant (not shown). In perfused regions, StringDB analysis indicated clusters including ECM organization, oxidative phosphorylation, and actin organization. Proteins enriched in oxidative phosphorylation and actin organization clusters were generally downregulated in ECM particle-injected mice, and proteins enriched in the ECM organization cluster were upregulated (not shown). These proteomics data support physiological findings, indicated by increased coronary angiogenesis in ischemic myocardium, reduced infarct size, and increased myocardial contractility as shown by increased fractional shortening. Interestingly, perfused region proteomic changes suggest that ECM particle effects are transduced across the heart, here indicating a reduced hypertrophic burden by decreased ECM organization, actin organization, and oxidative phosphorylation. Finally, interaction clusters represented by both ECM particle proteins and differential heart proteins point toward potential mechanisms by which ECM particles mediate their effect on ischemic myocardium.

In preparations of ECM particles, COL1A1, COL1A2, and COL3A1 were the most abundant matrisome proteins by intensity as detected by LC-MS/MS. Other collagens and matrisome components including glycoproteins and proteoglycans were also measured with high abundance. We detected 147 matrisome proteins among ECM particle batches—about half of the number of known matrisome proteins. One hundred twelve matrisome proteins were detected with confidence (RSD 20%) in Batch 1, 121 in Batch 2, and 82 in Batch 3. Eighty proteins were detected with confidence in all three batches. Matrisomes of the ECM particles were consistent among batches, with 27 proteins significantly different (P value 0.05) comparing Batch 1 versus Batch 2, 17 proteins comparing Batch 1 versus Batch 3, and only 6 proteins between Batch 2 and Batch 3. Together, these data show that ECM particles represent human ECM and are consistent from batch to batch.

Discussion: Our results demonstrate that intramyocardial delivery of ECM particles results in significant improvements in cardiac function as evidenced by increased fractional shortening (FS), decreased infarct size, and increased capillary density in ischemic myocardium in mice injected with ECM particles when compared with vehicle-treated controls in post-MI animals. There were also strong trends in improvements of arteriolar density in ischemic myocardium, but it did not reach statistical significance. Ejection fraction (EF) in ECM treated heart did not differ significantly from the nontreated heart. Given the significant improvement in FS, infarct size, and capillary density, there may be a localized trophic effect of ECM particles in the setting of acute myocardial ischemia. This is further supported by our biodistribution study that demonstrates that ECM particles delivered by I/M injections remain localized at the point of injection and immediate vicinity.

Proteomic analyses of post-MI heart tissue revealed changes in protein expression in ischemic and perfused regions that support improved cardiac function associated with ECM particle injection. Proteins involved in processes including cardiac muscle contraction and fatty acid b-oxidation were upregulated in ischemic myocardium injected with ECM particles, whereas proteins involved in oxidative phosphorylation were downregulated. Increased expression of proteins involved in oxidative phosphorylation, including NDUFS3 and UQCRQ, and decreased fatty acid oxidation have been reported in patients with heart failure. Our findings that ECM injected ischemic myocardium showed increased expression of proteins involved in fatty acid oxidation and decreased expression of oxidative phosphorylation proteins indicate recovery of cardiac cell metabolism homeostasis. Proteins involved in cardiac muscle contraction, including troponins and myosins, were upregulated in less soluble fractions of ECM particle-injected heart tissue from ischemic regions, suggesting a more robust architecture of contractile cells. Although collagen and collagen-regulating protein levels were unchanged in ischemic regions, they were increased in perfused regions of ECM particle-injected hearts. Since our study design did not include a control group without myocardial infarction, we were unable to determine if this change is due to increased collagen synthesis or decreased collagen degradation. However, together with decreased infarct size and increased fractional shortening associated with ECM particle injection, this indicates a return to contractile homeostasis communicated throughout the heart by interaction of particles with resident cells in the infarct region. This is further supported by high abundance of proteins involved in ECM and actin organization found in ECM particles, which may link to actinomyosin machinery through ECM particle proteins such as vinculin (VCL) or vimentin (VIM). Finally, both up- and downregulations of cytoskeleton proteins were detected in perfused regions of ECM particle injected hearts. Cytoskeleton regulation is perturbed in heart failure, indicating the need for more specific assessment of actin regulation mechanisms affected by ECM particle injection, such as post-translational modification status. Together, proteomic analyses support our physiological data as well as histological findings that ECM particles exerted beneficial effects on recovery of post-MI ischemic myocardium. ECM particle injection in the ischemic region appears to stabilize cellular phenotype, leading to improvement in vascular density, and thus attenuation of processes leading to adverse remodelling and heart failure after MI. This early recovery in ischemic myocardium by injection of ECM particles, as suggested by improvement in fractional shortening (FS) as early as POD7, might also have an effect of decreased compensatory pressure on the rest of the heart resulting in the prevention of adverse remodelling in the post-MI heart. There is a large body of evidence demonstrating that treatment with exogenous decellularized ECM (dECM) in the setting of cardiac ischemia is beneficial. However, the spectrum of results is as heterogenous as the sources and modifications made to the dECM itself. Porcine myocardial dECM injections in a swine MI model and patches in a rat MI model appear to increase cardiac muscle mass and improve EF and other echocardiographic parameters of function. Yet porcine bone marrow derived dECM only appeared to improve infarct size in a rat ischemia reperfusion model with the addition of methylcellulose, and porcine small intestinal submucosal dECM with or without the addition of circulating angiogenic cells used in a mouse MI model demonstrated significant improvements in both infarct size and EF. Injection of human dECM derived from placental tissue has also been studied and shown to reduce infarct size in a rat MI model. Injection with our laboratory grown ECM particles is consistent with these results and demonstrated not only decreased infarct size but also improved wall motion via FS at the point of injection. These results support the paramount importance of the ECM microenvironment, and further intimate that the source tissue of ECM appears to have a profound effect on surrounding tissue.

The localized improvement in wall motion as evidenced by improved FS may be in part explained by the increased capillary density observed within the infarcted region in the mice injected with ECM particles. This finding is consistent with dECM treatment in murine and rat models of myocardial injury leading to increased vascular density. There is also strong evidence at the cellular level that the ECM microenvironment has a profound effect on angiogenesis, with prior studies demonstrating that exposure to dECM increases VEGF-R2 and CD31 expression in human cardiac progenitor cells and increases HUVEC cell migration and tube formation. Furthermore, although it did not reach significance, we noted a strong trend toward increased arteriolar density in the infarcted region. Other groups have demonstrated significant increases in cardiac arteriolar density after injection of dECM in both rats and swine. Given the heterogeneity of native tissue used to source ECM in various experiments, it is understandable that results vary from study to study. In fact, multiple studies have demonstrated significant differences when directly comparing dECM from different source tissue including significant differences in proliferation and migration of endothelial and smooth muscle cells. Findings like these illustrate the need for a customizable, scalable, and reproducible, laboratory-grown source material for ECM therapy, and underscore our rationale for using human MSCs from umbilical cords as we are able to leverage their regenerative potential and well-established methods to scale-up the production of MSCs needed for a therapy.

We observed no significant changes to proliferation or apoptosis in any region (ischemic, border, and remote regions) in post-MI hearts. This lack of difference in apoptosis is unsurprising given our single data point for immunohistochemistry is at post-MI day 28. It is plausible that at early time points (POD 3, 7, 14), there might have been significant differences in apoptosis and/or proliferation activities between ECM particle-injected and control hearts. Similarly, as we are using an adult mouse model and the mature myocardium does not regenerate, it is not surprising that no difference in the number of Ki-67 positive cells was observed. Although the mature myocardium is quiescent, porcine dECM has been demonstrated to facilitate the proliferation of cardiac progenitor cells in vitro. Interestingly, however, this is not true of all groups. In one group, treatment of MI mice with dECM microparticles significantly increased the amount of Ki-67 positive myocytes when compared with controls. Similarly porcine dECM has been shown to increase cell proliferation in vitro. This same group demonstrated in a rat MI model that a methylcellulose dECM treatment reduced apoptosis compared with controls. Another group demonstrated that porcine dECM was able to significantly reduce expression of BCL-2 by qPCR in an ischemia-reperfusion rat model, but no significant difference was seen in myocytes expressing caspase-3. However, our data do not include early time points in the recovery phase (POD 3, 7), and thus we cannot exclude possibility of modulation in Ki-67 activities in endothelial or other vascular cells at early time points. Similar reasons may have precluded detection of any differences in TUNEL staining between control and ECM-injected ischemic myocardium as the early time point remodelling involving cell death and/or proliferation was likely completed by the time the hearts were harvested (POD 28).

Although decellularized ECM certainly has reduced immunogenicity compared with whole tissue, immune response is a valid concern in any xenotransplantation of cellular elements. This was the driving force behind our selection of human mesenchymal stem cells for our laboratory grown ECM particles. In one rat MI model study using porcine dECM, transcriptomic profiling showed reduced CD68 levels; however, tissue staining revealed no difference in macrophage expression. Interestingly, another group showed that CD68 expression was upregulated in the ECM injection cohort via qPCR, whereas again no difference in CD68 positive cells were seen on histologic analysis. Yet another group performing dECM injections in rats observed no difference in M2 macrophage expression via IHC of CD163 between ECM and controls. In our study, with the exception of increased M2 macrophage expression in the perfused region, no difference in immune response was appreciated between the cohort receiving ECM particles and controls suggesting a low immunogenicity.

The time course of ECM treatment has been of great interest to the research community as it has been established that traditional ECM hydrogels can degrade within 7 days of administration. Recent attempts to overcome this limitation by injecting decellularized solid microparticles of dECM rather than ECM hydrogels have been met with some success. Specifically, one study examining dECM microparticles in a murine MI model showed significantly increased ECM retention in the microparticle group compared with mice treated with ECM hydrogel. However, both the dECM microparticle and ECM hydrogel arms of this study appeared to have similar functional outcomes at 3 wk post-MI with no significant difference in EF or FS demonstrated. Interestingly, our novel human stem cell-derived 3-D ECM particles appear to buck the trend of diminishing effect over time seen in other studies. Given the time course of wound remodelling, in which transition from type III to type I collagen occurs 7-10 days post-insult, and the functional differences we observed after day 7, we hypothesize that ECM particles, which have collagen type I to type III ratio similar to the human heart, may facilitate the wound remodelling process by providing a stable niche that directs adjacent cells toward healthy repair. Given this, future studies investigating the degradative time course of our ECM particles is warranted. Ongoing studies in our laboratory include confirmation of the major proteome pathways affected by ECM, namely proteins involved in myocardial contractility and metabolism in post-MI ischemic myocardium.

The relative sensitivity of the murine heart to large volume fluctuations and limited injection area may have diminished the effect of our human 3-D ECM particles on EF. However, given the local effects demonstrating significantly improved FS and LV infarct size as well as remarkable improvement in capillary density in ischemic myocardium injected with ECM particles, we anticipate that improved wall kinetics over a larger area in a large animal model would likely have a more profound effect on overall EF than the initial minimal trend toward improvement observed in our current study using mice. Sample size used in our study was calculated using power analysis and previous experiences. However, the study was limited by sample size and may have been underpowered to detect significant changes in EF and arteriolar density. Despite these shortcomings, the data presented in the current study are promising and raises questions about the impact of 3-D human ECM particles in the days following acute MI and their translatability to human heart disease. To address these, we propose the following future research directions. First, the effects of treatment with 3-D ECM particles in the acute post-MI phase at early time points merit investigation. Second, investigating the effects of ECM particles in a high-fidelity clinically relevant large animal model of chronic ischemia.

Finally, given the rich complexity of the ECM particles, it is plausible that ECM are acting in a multifunctional manner, and future studies are needed to determine the key attributes of the ECM particles and their precise mechanism(s) of action. Conclusions Intramyocardial injection of laboratory-grown acellular 3-D human ECM particles derived from stem cell spheroids significantly improved coronary angiogenesis, cardiac FS, and corresponding significant decrease in infarct size in post-MI hearts. Although there was a trend toward increased arteriolar density in ECM-injected ischemic myocardium, improvement in coronary angiogenesis consisted mostly of significant increase in capillary density. Interestingly, 3-D ECM injected hearts showed an initial trend in the improvement in EF (at POD 7), which was not evident at later time points (POD 21 or POD 28) and never reached significance. These results likely represent a profound localized effect of 3-D human ECM particles on injured myocardial tissue, further supported by proteomic analyses showing increased muscle contraction and fatty acid b-oxidation proteins in the ischemic region. Furthermore, our laboratory-grown acellular 3-D human ECM particles yielded similar effects to various dECM counterparts in literature. This strategy of laboratory-grown ECM production using human MSCs rather than harvesting from animal tissues or modification by other means, liberates one from the limitations of the properties and quantities of the tissue from which ECM can be harvested. It also allows for customization of the ECM formulation for specific targets, enhanced reproducibility, lower immunogenicity, and potential for scalability.

Brief Additional Description of Materials and Methods; ECM particles; ECM particles can be produced, in brief, as described in Example 1.

### Assessment of the Effects of ECM Particles on Cardiac Function and Morphology

ECM particles were suspended at a concentration of 63,400 particles per mL to create an injectable solution. FVB mice underwent left anterior descending (LAD) artery ligation followed by intramyocardial injection of 10 μL of saline control (n=10) or ECM solution (n=10) to the infarcted area. Echocardiography was performed pre-op and on post-op days 3, 7, 14, 21, and 28 in order to determine ejection fraction (EF) and fractional shortening (FS). On post-op day-28 (POD28), mice were euthanized and cardiac tissue was collected for proteomic analysis, immunohistochemistry, and immunofluorescent microscopy.

### Results: Effects of MSC ECM Particles on Key Remodeling Regulators

FIGS. 18A-18D show molecular factors of adverse remodeling after myocardial infarction (MI) and factors for healthy recovery provided by ECM particles injected after MI. Molecular factors of adverse remodeling after the MI include: (i) a decreased Collagen I/Collagen III ratio (COL1/COL3); (ii) a Collagen V (COL5) deficiency and an increased integrin A5 (ITGAV) activity; (iii) enhanced TGFβ signaling; and (iv) increased endotrophin levels from Collagen α3(VI) (COL6A3) cleavage.

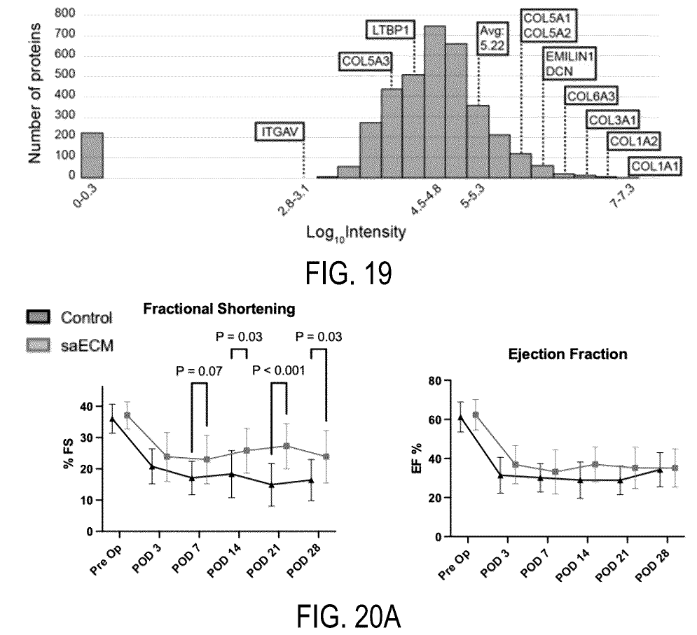

Injection of MSC ECM particles after the MI provides factors to the heart for a healthy recovery. Injection of MSC ECM particles led to: (i) a restoration of the COL1/COL3 ratio to physiological levels (FIG. 18A); (ii) a high level of COL5, which inhibited ITGAV activity, resulting in smaller scars (FIG. 18B); (iii) decorin (DCN), emilin1, and latent transforming growth factor β binding proteins (LTBPs) sequestered TGFβ, thereby reducing TGFβ signaling (FIG. 18C); and (iv) cleavage of endotrophin from the MSC ECM particles (FIG. 18D). Intensities of these proteins are represented by the histogram profile of MSC ECM particle proteins shown in FIG. 19.

### MSC ECM Particles Improve Cardiac Function and Morphology in Myocardial Ischemia

Treatment with ECM particles resulted in significant improvement in FS after POD14 that continued to POD28 (p<0.05 for all). A similar trend in improved EF was observed but failed to reach statistical significance (FIG. 20A). Infarct size was significantly reduced in ECM treated mice compared with controls (p=0.04) (FIG. 20B). Within the infarcted region there was a trend towards increased arteriolar density in ECM treated mice (p=0.12) and a significant increase in capillary density (p=0.01). No significant difference was observed in arteriolar or capillary density in the infarct border zone or remote non-ischemic area (p>0.05 for all) (FIG. 20C). No significant difference was observed in ki67, TUNEL, TGFβ, or 3-nitrotyrosine staining in any heart region (p>0.05 for all).

Conclusion/Discussion: ECM particles produced from decellularized 3D microtissues of human mesenchymal stem cells were injected into a mouse model of heart failure and shown to have beneficial effects as measured by an increase in fractional shortening, a decrease in infarct scar size and an increase in capillaries/arterioles. The ability to precisely control the construct of ECM particles represents a potential therapeutic strategy for the treatment of myocardial ischemia, heart failure, and or other indications where scarring/fibrosis is problematic. The treatment of heart failure and other indications where scarring and fibrosis are problematic are major unmet medical needs. ECM particles are potentially a new regenerative therapy to treat diseases whose underlying pathology is multi-factorial. The promise of injection of extracellular matrix (ECM) from animal hearts as a treatment of myocardial ischemia has been limited by immune reactions and harsh ECM-damaging extraction procedures. We developed a novel method to produce lab-grown human three-dimensional (3-D) acellular ECM particles from human mesenchymal stem cells (MSCs) to mitigate product variability, immunogenicity, and preserve ECM architecture.

We hypothesized that intramyocardial injection (I/M) of this novel ECM (dia 200 microns) would improve cardiac function in a post myocardial infarction (MI) murine model. WT mice aged 8-10 wk underwent ligation of the left anterior descending coronary (LAD) artery and I/M injection of 10 μL ECM or normal saline (n=10/group). Compared with control, ECM-treated hearts showed significant reduction in infarct size (P=0.04), increased capillary density in ischemic myocardium (P=0.01), and increased fractional shortening (FS) (P<0.05) on postoperative days (POD) 14, 21, and 28 by echocardiography. There were no significant differences in immunogenic response as determined by TNFa, IL6, CD86, or CD163 levels (P>0.05 for all) in the hearts. Biodistribution of fluorophore-conjugated ECM demonstrated localized epifluorescence in the heart after I/M injection, without significant peripheral end organ epifluorescence. Proteomic analysis of is chemic and perfused myocardium from control and ECM-treated hearts using LC-MS/MS (n=3/group) detected significant changes in proteins involved in cardiomyocyte contractility and fatty acid metabolism. These findings suggest that 3-D ECM particles induce recovery of ischemic myocardium, by upregulating protein networks involved in cellular contractility and metabolism. Taken together, 3-D ECM particles represent a promising therapy for MI and warrant confirmatory studies in a high-fidelity large animal model.

### Example 15. Altering the Proteome of a Diseased Heart (or Organ)

First, the following examples, any of which can be inter-combined with any detail, feature, example, embodiment, figure, reference, aspect, or disclosure herein are repeated and tested:

Test example 1: A non-adhesive microwell including a cell culture medium and about 500 to about 4000 seeded human heart cells configured in a composition, the composition consisting of both 1) human heart tissue spheroids and 2) porous decellularized human heart ECM (extracellular matrix) spheres; wherein the human heart tissue spheroids 1) comprise a diameter ≤300 μm and each spheroid includes self-adherent human heart cells grown from the seeded cells seeded in the non-adhesive microwell and grown into a 3D fibrous network architecture with a 3D spheroid shape, without an adherence to an adherent growth surface and without added growth scaffold material; then at least a portion of the ≤300 μm spheroids are decellularized, to leave behind 2); wherein the porous spheres of decellularized human heart ECM 2) include a maintained 3D spheroid-shape from 1) and the 3D fibrous network architecture of the spheroids 1); wherein the porous ECM spheres 2) have a diameter ≤800 μm; and wherein the composition is in contact with at least one of: a detergent, a sterile phosphate buffered saline (PBS), a DNase and/or an RNase, a transforming growth factor beta (TGF-β), an interleukin 4, an interleukin 10, an interleukin 11, or an interleukin 13.

Test example 2: A method of producing decellularized human heart extracellular matrix (ECM) morsels, the method comprising: seeding cultured human heart cells into micro-wells, wherein the cultured human heart cells generate an ECM microtissue comprised of the cultured cells and ECM produced from the cultured cells; collecting the ECM microtissue as spheroid particles; and decellularizing the ECM microtissue; wherein said decellularized ECM morsels comprise a physical or chemical composition differing from the physical or chemical composition of in-vivo derived adult human heart ECM in collagen and sulphated glycosaminoglycans (sGAG) content.

Test example 3: The method of test example 2, wherein said decellularizing comprises mixing the microtissue with at least one of a detergent, a buffer, a DNAse, or an RNase. Test example 4: The method of test example 2, wherein said ECM microtissue comprises spherical particles having a diameter of less than or equal to 800 μm. Test example 5: The method of test example 2, wherein said cells comprise recombinant human cells. Test example 6: The method of test example 2, wherein said cells comprise at least one of cardiac fibroblasts, cardiac myocytes and cardiac microvascular endothelial cells.

Test example 7: A non-adhesive tube or a non-adhesive container including a cell culture medium and at least 500 seeded human heart cells configured in a composition, the composition consisting of both 1) human heart tissue spheroids and 2) porous decellularized human heart ECM (extracellular matrix) spheres; wherein the human heart tissue spheroids 1) comprise a diameter ≤300 μm and each spheroid includes self-adherent human heart cells grown from the seeded cells seeded in the non-adhesive tube or container, grown into a 3D fibrous network architecture with a 3D spheroid shape, without an adherence to an adherent growth surface and without added growth scaffold material; then at least a portion the ≤300 μm spheroids are decellularized, to leave behind 2); wherein the porous spheres of decellularized human heart ECM 2) include a maintained 3D spheroid-shape from 1) and the 3D fibrous network architecture of the spheroids 1); wherein the porous ECM spheres 2) have a diameter ≤800 μm; and wherein the composition is in contact with at least one of: a detergent, a buffer, a DNase and/or an RNase, a transforming growth factor beta (TGF-β), an interleukin 4, an interleukin 10, an interleukin 11, or an interleukin 13.

Test example 8: The tube or container of test example 7, wherein the composition is in contact with sterile phosphate buffered saline (PBS), a detergent, and a DNAse or an RNase.

Test example 9: The tube or container of test example 8, wherein the tube or container is in a form of a syringe with a needle attached to the syringe. Test example 10: The tube or container of test example 9, wherein the syringe comprises a 27 G needle and wherein one or more ECM spheres that are operative to pass through the 27 G needle each include a diameter of less than about 200 μm. Test example 11: The tube or container of test example 7, further comprising wherein said human heart cells comprise recombinant human cells.

Test example 12: The tube or container of test example 7, further comprising wherein said human heart cells comprise cardiac fibroblasts, cardiac myocytes, cardiac microvascular endothelial cells, or a combination thereof.

Test example 13: The method of test example 2, wherein said micro-wells do not comprise an added growth scaffold material.

Test example 14: The tube or container of test example 7, with a proviso that the tube or container does not comprise an added growth scaffold material.

Test example 15: The method of test example 2, wherein said modifying comprises adjusting: cell culture media composition, culturing time, oxygen level, or the presence or amount of additional biological factors.

Test example 16: The method of test example 2, wherein said additional biological factors comprise a growth factor, a cytokine, or a drug.

Test example 17: A method of producing decellularized human heart extracellular matrix (ECM) morsels, the method comprising: seeding cultured human heart cells into micro-wells, wherein the cultured cells generate an ECM microtissue comprised of the cultured cells and an ECM, wherein said ECM microtissue comprises spherical particles having a diameter of less than or equal to 800 μm; collecting the ECM microtissue; and decellularizing the ECM microtissue; wherein said decellularized human heart ECM morsels comprise a physical or chemical composition differing from the physical or chemical composition of in-vivo derived adult human heart ECM in collagen and sulphated glycosaminoglycans (sGAG) content.

Test example 18: The non-adhesive microwell of test example 1, wherein the range of the mechanical stiffness of the ECM spheres is lower and higher than the range of mechanical stiffness of healthy human heart tissue.

Test example 19: The method of test example 2, wherein the mechanical stiffness of the ECM is comparable to healthy human heart tissue.

Test example 20: The tube or container of test example 7, wherein the range of the mechanical stiffness of the ECM spheres is lower and higher than the range of mechanical stiffness of healthy human heart tissue.

Test example 21: The method of test example 17, wherein the range of the mechanical stiffness of the ECM spheres is lower and higher than the range of mechanical stiffness of healthy human heart tissue.

Test example 22: The non-adhesive microwell of test example 1, wherein a majority of the ≤300 μm spheroids are decellularized

Test example 23: The tube or container of test example 7, wherein a majority of the ≤300 μm spheroids are decellularized.

Test example 24: The non-adhesive microwell of test example 1, further comprising wherein the composition is in contact with at least one of: collagen, one or more sulfated glycosaminoglycans (sGAG), or less than 50 ng/mg (wt/wt) dry weight of a double-stranded (dsDNA).

Test example 25: The tube or container of test example 7, further comprising wherein the composition is a flowable composition, wherein the flowable composition is a composition that can be administered with a cannula or a needle.

Test example 26: The non-adhesive microwell of test example 1, wherein about all of the ≤300 μm spheroids are decellularized.

Test example 27: The non-adhesive microwell of test example 1, wherein the composition is a flowable composition, wherein the flowable composition is a composition that can be administered with a cannula or a needle.

Test example 28: A non-adhesive microwell including: a cell culture medium; about 500 to about 4000 seeded human heart cells grown in a form of a composition, the composition consisting of both 1) human heart tissue spheroids and 2) porous decellularized human heart ECM (extracellular matrix) spheres; wherein the human heart tissue spheroids 1) comprise: a diameter ≤300 μm, a 3D fibrous network architecture, and a 3D spheroid shape; wherein the porous spheres of decellularized human heart extracellular matrix (ECM) 2) comprise: the 3D spheroid-shape, the 3D fibrous network architecture, and a diameter ≤800 μm; wherein the composition is in contact with at least one of: a detergent, a sterile phosphate buffered saline (PBS), a DNase and/or an RNase, a transforming growth factor beta (TGF-β), an interleukin 4, an interleukin 10, an interleukin 11, or an interleukin 13.

Test example 29: A method of producing decellularized human heart extracellular matrix (ECM) morsels, the method comprising: seeding cultured human heart cells into micro-wells, wherein the cultured human heart cells generate an ECM microtissue comprised of the cultured cells and ECM produced from the cultured cells; collecting the ECM microtissue as spheroid particles; and decellularizing the ECM microtissue; wherein said decellularized ECM morsels comprise a physical or chemical composition differing from the physical or chemical composition of in-vivo derived adult human heart ECM in collagen and sulphated glycosaminoglycans (sGAG) content.

Test example 30: The method of test example 29, wherein said decellularizing comprises mixing the microtissue with at least one of a detergent, a buffer, a DNAse, or an RNase.

Test example 31: The method of test example 29, wherein said ECM microtissue comprises spherical particles having a diameter of less than or equal to 800 μm.

Test example 32: The method of test example 29, wherein said cells comprise recombinant human cells.

Test example 33: The method of test example 29, wherein said cells comprise at least one of cardiac fibroblasts, cardiac myocytes and cardiac microvascular endothelial cells.

Test example 34: A non-adhesive tube or a non-adhesive container comprising: a cell culture medium: at least 500 seeded human heart cells grown in a form of a composition, the composition consisting of both 1) human heart tissue spheroids and 2) porous decellularized human heart ECM (extracellular matrix) spheres; wherein the human heart tissue spheroids 1) comprise: a diameter ≤300 μm, a 3D fibrous network architecture, and a 3D spheroid shape; wherein the porous spheres of decellularized human heart extracellular matrix (ECM) 2) comprise: the 3D spheroid-shape, the 3D fibrous network architecture, and a diameter ≤800 μm; wherein the composition is in contact with at least one of: a detergent, a buffer, a DNase and/or an RNase, a transforming growth factor beta (TGF-β), an interleukin 4, an interleukin 10, an interleukin 11, or an interleukin 13.

Test example 35: The tube or container of test example 34, wherein the composition is in contact with sterile phosphate buffered saline (PBS), a detergent, and a DNAse or an RNase.

Test example 36: The tube or container of test example 35, wherein the tube or container is in a form of a syringe with a needle attached to the syringe.

Test example 37: The tube or container of test example 36, wherein the syringe comprises a 27 G needle and wherein one or more ECM spheres that are operative to pass through the 27 G needle each include a diameter of less than about 200 μm.

Test example 38: The tube or container of test example 34, further comprising wherein said human heart cells comprise recombinant human cells.

Test example 39: The tube or container of test example 34, further comprising wherein said human heart cells comprise cardiac fibroblasts, cardiac myocytes, cardiac microvascular endothelial cells, or a combination thereof.

Test example 40: The method of test example 29, wherein said micro-wells do not comprise an added growth scaffold material.

Test example 41: The tube or container of test example 34, with a proviso that the tube or container does not comprise an added growth scaffold material.

Test example 42: The method of test example 29, wherein said modifying comprises adjusting: cell culture media composition, culturing time, oxygen level, or the presence or amount of additional biological factors.

Test example 43: The method of test example 29, wherein said additional biological factors comprise a growth factor, a cytokine, or a drug.

Test example 44: A method of producing decellularized human heart extracellular matrix (ECM) morsels, the method comprising: seeding cultured human heart cells into micro-wells, wherein the cultured cells generate an ECM microtissue comprised of the cultured cells and an ECM, wherein said ECM microtissue comprises spherical particles having a diameter of less than or equal to 800 μm; collecting the ECM microtissue; and decellularizing the ECM microtissue; wherein said decellularized human heart ECM morsels comprise a physical or chemical composition differing from the physical or chemical composition of in-vivo derived adult human heart ECM in collagen and sulphated glycosaminoglycans (sGAG) content.

Test example 45: The non-adhesive microwell of test example 28, wherein the range of the mechanical stiffness of the ECM spheres is lower and higher than the range of mechanical stiffness of healthy human heart tissue.

Test example 46: The method of test example 29, wherein the mechanical stiffness of the ECM is comparable to healthy human heart tissue.

Test example 47: The tube or container of test example 34, wherein the range of the mechanical stiffness of the ECM spheres is lower and higher than the range of mechanical stiffness of healthy human heart tissue.

Test example 48: The method of test example 44, wherein the range of the mechanical stiffness of the ECM spheres is lower and higher than the range of mechanical stiffness of healthy human heart tissue.

Test example 49: The non-adhesive microwell of test example 28, further comprising wherein a majority of the ≤300 μm spheroids are decellularized

Test example 50: The tube or container of test example 34, further comprising wherein a majority of the ≤300 μm spheroids are decellularized.

Test example 51: The non-adhesive microwell of test example 28, further comprising wherein the composition is in contact with at least one of: collagen, one or more sulfated glycosaminoglycans (sGAG), or less than 50 ng/mg (wt/wt) dry weight of a double-stranded (dsDNA).

Test example 52: The tube or container of test example 34, wherein the composition is a flowable composition, wherein the flowable composition is a composition that can be administered with a cannula or a needle.

Test example 53: The non-adhesive microwell of test example 28, wherein about all of the ≤300 μm spheroids are decellularized.

Test example 54: The non-adhesive microwell of test example 28, wherein the composition is a flowable composition, wherein the flowable composition is a composition that can be administered with a cannula or a needle.

Test example 55: A method of altering the proteome of an organ, tissue and/or a diseased organ, comprising: contacting the organ and/or tissue with extra-cellular matrix (ECM) particles produced from decellularized 3D microtissues, wherein the contacting alters the proteome of the organ and/or tissue.

Test example 56: The method of test example 55, wherein the contacting restores a normal ratio of levels of different collagens in the organ and/or tissue.

Test example 57: The method of test example 55, wherein the contacting alters an immune response in the organ and/or tissue.

Test example 58: The method of test example 55, wherein the contacting alters metabolism in the organ and/or tissue.

Test example 59: The method of test example 55, wherein the contacting mitigates effects of TGF-Beta in the organ and/or tissue, wherein TGF-Beta is a growth factor associated with fibrosis.

Test example 60: The method of test example 55, wherein the 3D microtissues are derived from human mesenchymal stem cells.

Test example 61: A method of treating an organ, tissue and/or a diseased organ, comprising: administering to the organ and/or tissue extra-cellular matrix (ECM) particles produced from decellularized 3D microtissues, wherein the administering treats the organ and/or tissue by altering the proteome of the organ and/or tissue.

Test example 62: The method of test example 61, wherein the administering restores a normal ratio of levels of different collagens in the organ and/or tissue.

Test example 63: The method of test example 61, wherein the administering alters an immune response in the organ and/or tissue.

Test example 64: The method of test example 61, wherein the administering alters metabolism in the organ and/or tissue.

Test example 65: The method of test example 61, wherein the administering mitigates effects of TGF-Beta in the organ and/or tissue, wherein TGF-Beta is a growth factor associated with fibrosis.

Test example 66: The method of test example 61, wherein the 3D microtissues are derived from human mesenchymal stem cells.

Test example 67: Extra-cellular matrix (ECM) particles for altering the proteome of an organ, tissue and/or a diseased organ, wherein the ECM particles are produced from decellularized 3D microtissues and treated to improve one or more of crosslinking, stability, injectability, or compatibility with a patient.

Test example 68: The ECM particles of test example 67, wherein the ECM particles, when contacted with the organ and/or tissue, restore a normal ratio of levels of different collagens in the organ and/or tissue.

Test example 69: The ECM particles of test example 67, wherein the ECM particles, when contacted with the organ and/or tissue, alter an immune response in the organ and/or tissue.

Test example 70: The ECM particles of test example 67, wherein the ECM particles, when contacted with the organ and/or tissue, alter metabolism in the organ and/or tissue.

Test example 71: The ECM particles of test example 67, wherein the ECM particles, when contacted with the organ and/or tissue, mitigate effects of TGF-Beta in the organ and/or tissue, wherein TGF-Beta is a growth factor associated with fibrosis.

Test example 72: The ECM particles of test example 67, wherein the 3D microtissues are derived from human mesenchymal stem cells.

Test example 73: A composition for treating an organ, tissue and/or a diseased organ, comprising: extra-cellular matrix (ECM) particles produced from decellularized 3D microtissues and treated to improve one or more of crosslinking, stability, injectability, or compatibility with a patient; wherein the composition, when administered to the organ and/or tissue, treats the organ and/or tissue by altering the proteome of the organ and/or tissue.

Test example 74: The composition of test example 73, wherein the 3D microtissues are derived from human mesenchymal stem cells.

Flowable compositions consisting of only 1) human heart tissue spheroids and 2) porous decellularized human heart ECM (extracellular matrix) spheres are produced; wherein the human heart tissue spheroids 1) comprise a diameter ≤300 μm and each spheroid includes self-adherent human heart cells grown from the seeded cells seeded in the non-adhesive microwell and grown into a 3D fibrous network architecture with a 3D spheroid shape, without an adherence to an adherent growth surface and without added growth scaffold material; then at least a portion of the ≤300 μm spheroids are decellularized, to leave behind 2); wherein the porous spheres of decellularized human heart ECM 2) include a maintained 3D spheroid-shape from 1) and the 3D fibrous network architecture of the spheroids 1); wherein the porous ECM spheres 2) have a diameter ≤800 μm. It is predicted to be found that these compositions consisting of 1) and 2) can be used in contact with formulations for human administration. After practicing the methods disclosed and testing the compositions, each method and each composition is tested on animal and alter human models with the purpose, or spirit of the invention being to save human lives.

Next, ECM particles produced from decellularized 3D microtissues of human mesenchymal stem cells were injected into a mouse model of heart failure. Four weeks after injection, sections of the mouse hearts were sent for proteomic analysis. A comparison of the proteomic data of the infarct site, a border site and a remote area showed that treatment with ECM particles helped restore the normal ratio of the levels of different collagens, altered the immune response, altered metabolism and mitigated the effects of a TGF-Beta, a growth factor associated with fibrosis. A novel method to alter the proteome of a diseased heart by injection of ECM particles produced from decellularized 3D microtissues of human mesenchymal stem cells.

These particles may be a method to treat heart failure and or other indications where scarring/fibrosis is problematic. These particles could be a product to treat disease.

The treatment of heart failure and other indications where scarring and fibrosis are problematic are major unmet medical needs. ECM particles are potentially a new regenerative therapy to treat diseases whose underlying pathology is multi-factorial.

### Proteome Data:

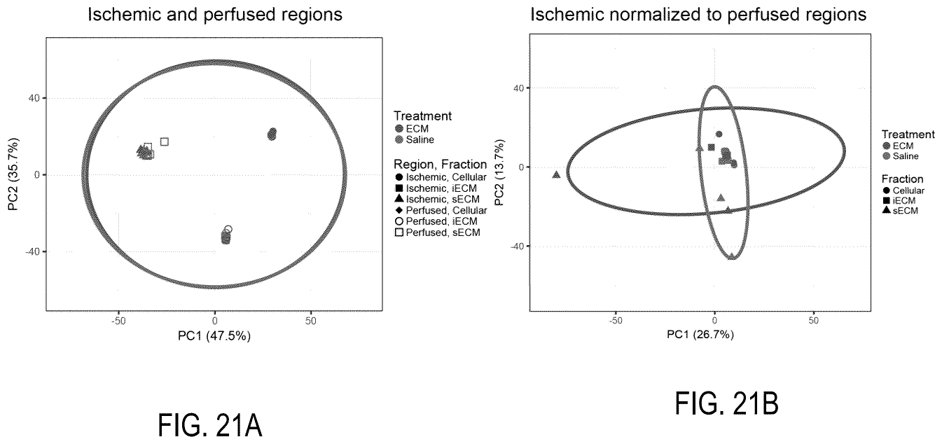

FIG. 21A shows a plot of Ischemic and perfused regions with PC1 (47.5%) vs. PC2 (35.7%). FIG. 21B shows a plot of Ischemic normalized to perfused regions with PC1 (26.7%) vs. PC2 (13.7%).

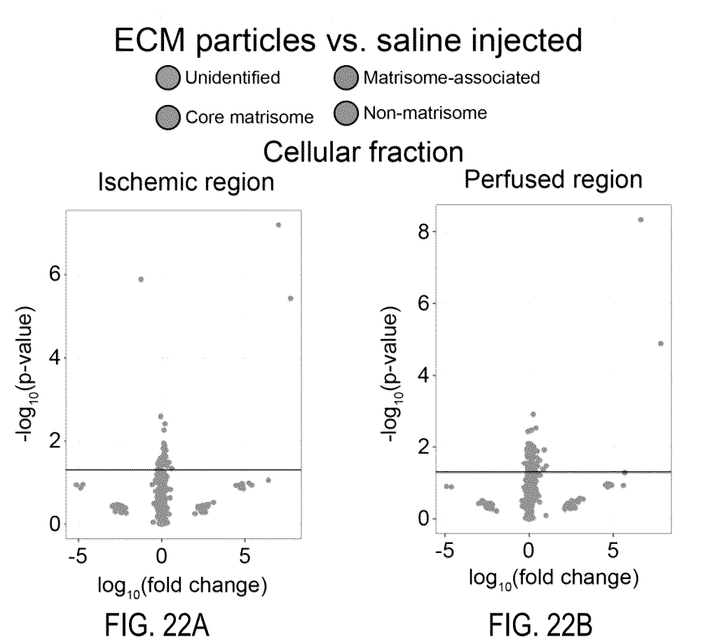

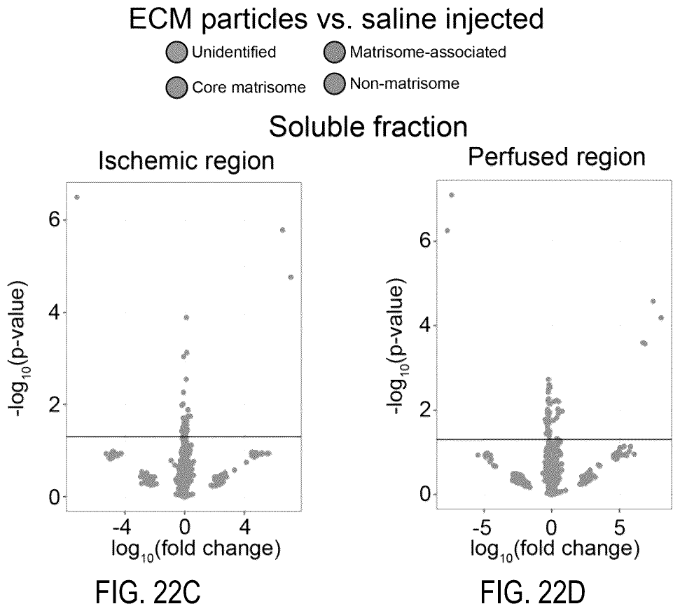

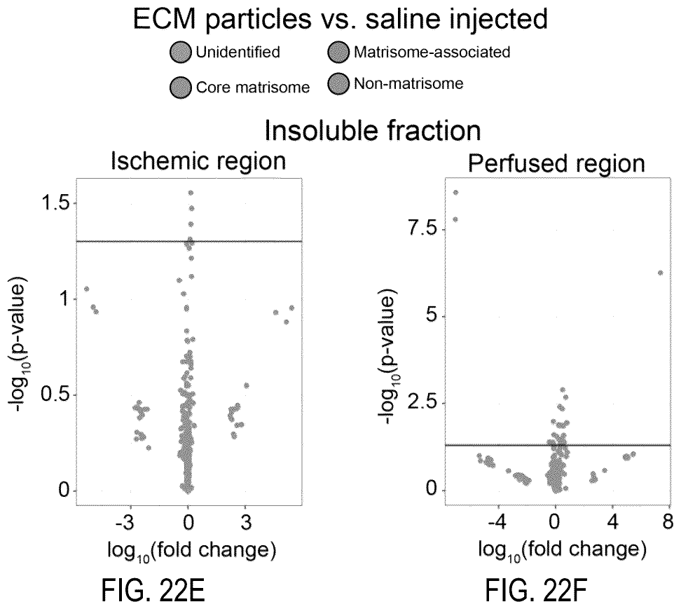

FIG. 22A shows a plot of Cellular fraction; Ischemic region ECM particles vs. saline injected. FIG. 22B shows a plot of Cellular fraction; Perfused region. FIG. 22C shows a plot of Soluble fraction; Ischemic region ECM particles vs. saline injected. FIG. 22D shows a plot of Soluble fraction; Perfused region. FIG. 22E shows a plot of Insoluble fraction; Ischemic region ECM particles vs. saline injected. FIG. 22F shows a plot of Insoluble fraction; Perfused region.

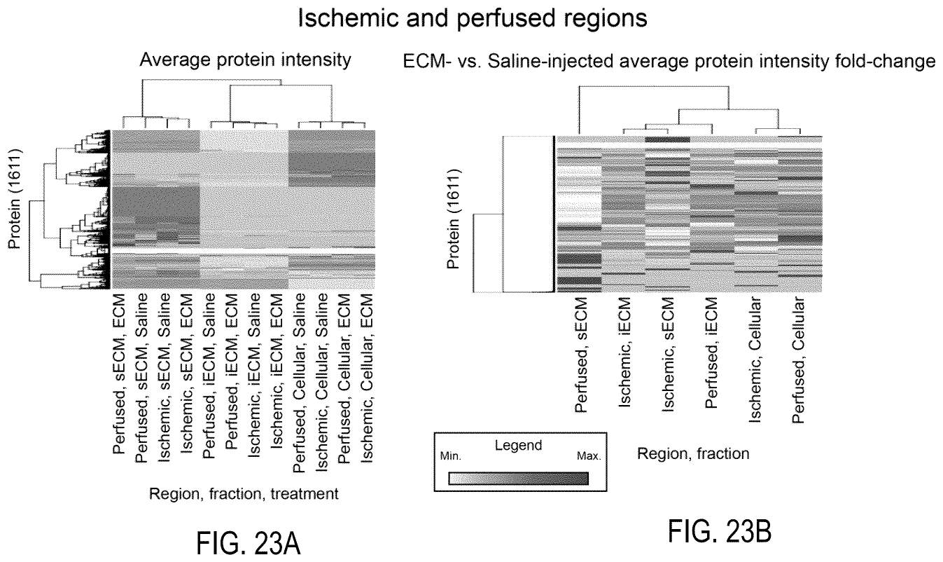

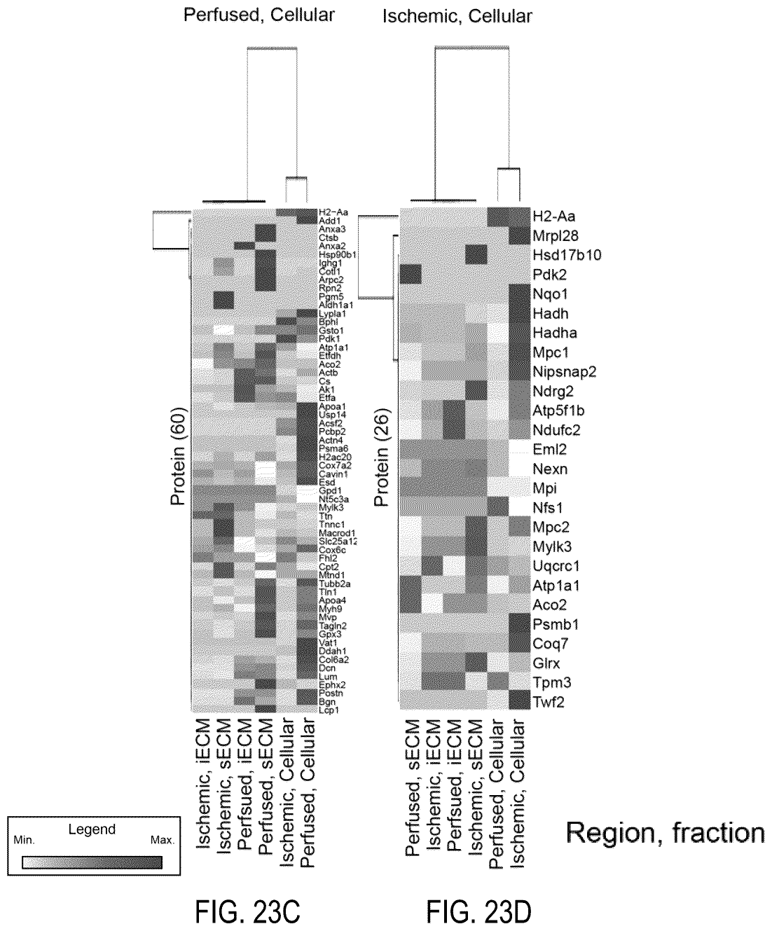

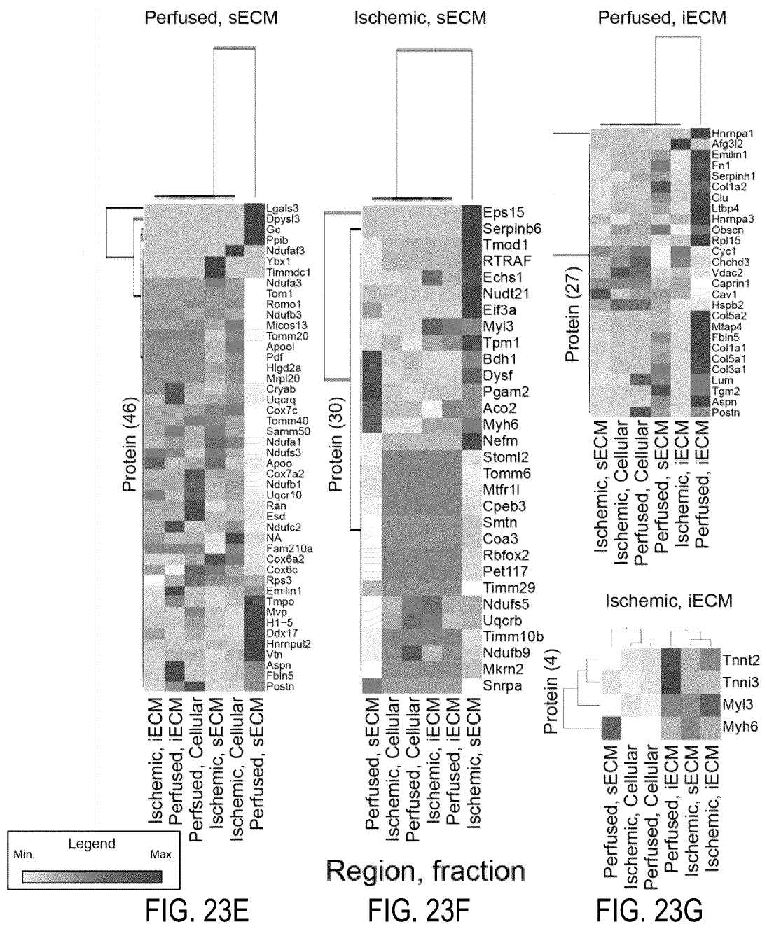

FIG. 23A shows Ischemic and perfused regions, Average protein density. FIG. 23B shows Ischemic and perfused regions, ECM-vs. Saline-injected average protein intensity fold-change. FIG. 23C shows ECM-vs. Saline-injected average protein intensity fold-change, p-value <0.5, for Perfused, cellular. FIG. 23D shows ECM-vs. Saline-injected average protein intensity fold-change, p-value <0.5, for Ischemic, cellular. FIG. 23E shows ECM-vs. Saline-injected average protein intensity fold-change, p-value <0.5, for Perfused, sECM. FIG. 23F shows ECM-vs. Saline-injected average protein intensity fold-change, p-value <0.5, for Ischemic, sECM. FIG. 23G shows ECM-vs. Saline-injected average protein intensity fold-change, p-value <0.5, for Perfused, iECM (top) and for Ischemic, iECM (bottom).

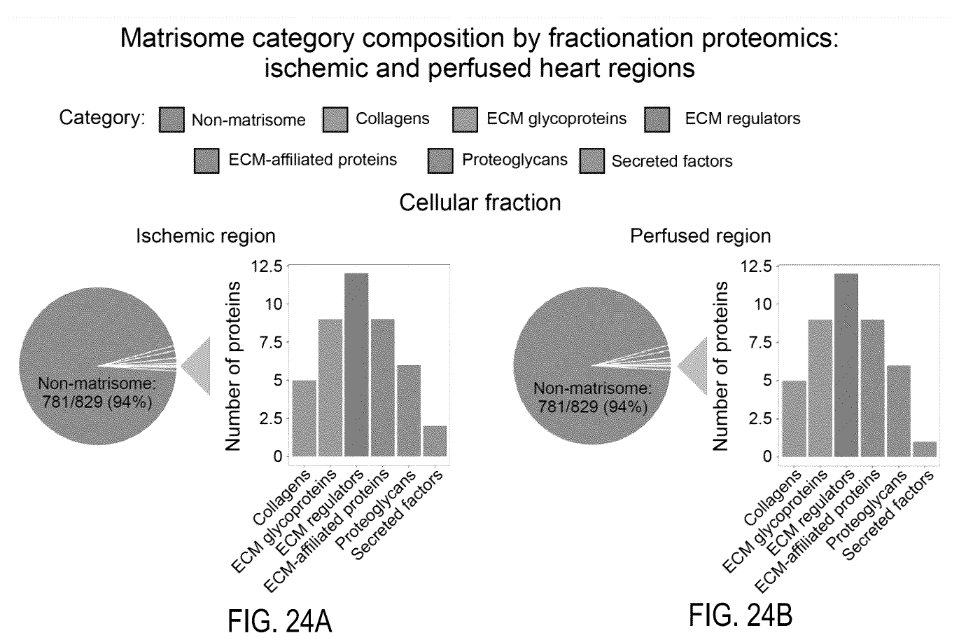

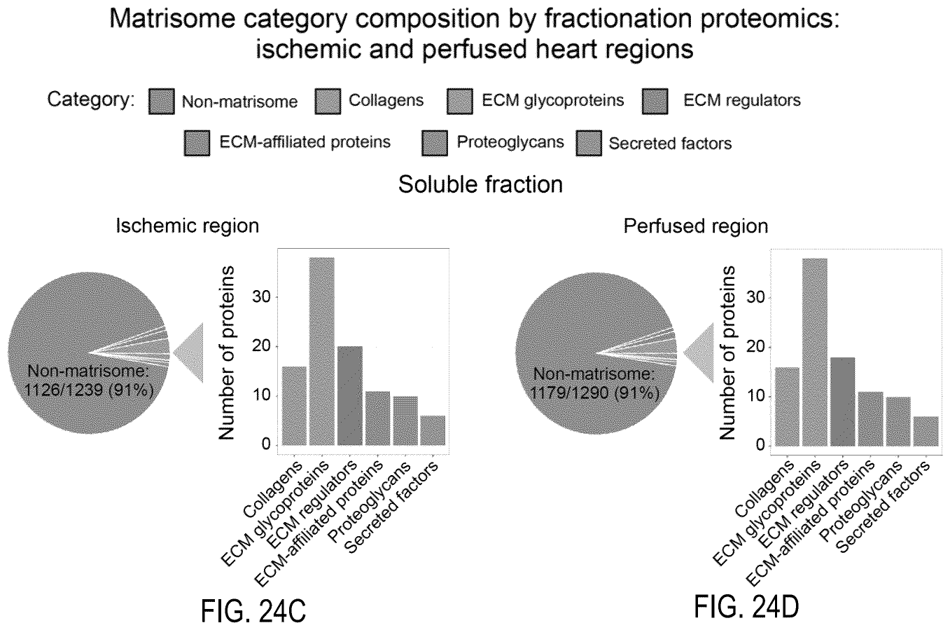

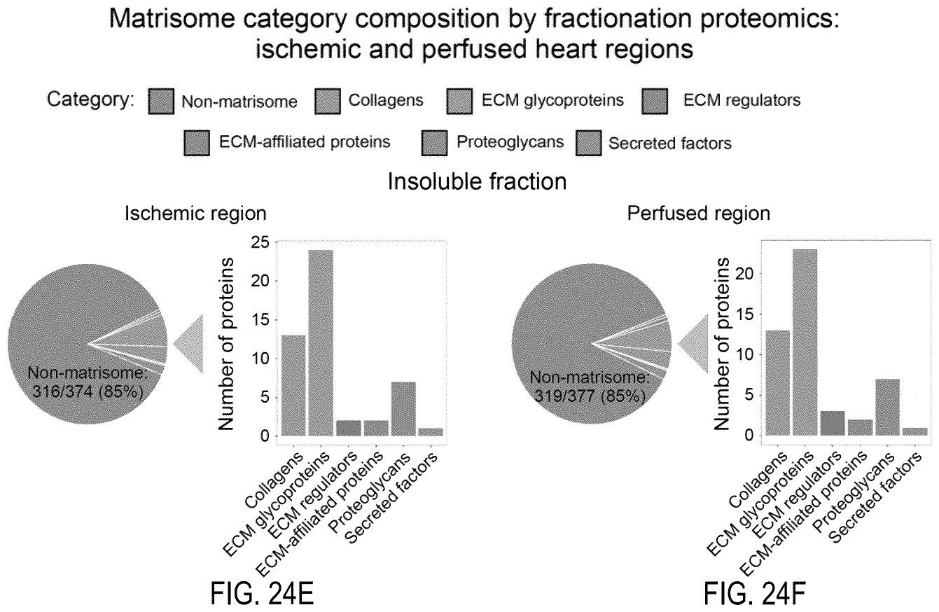

For the mouse heart proteomics study ischemic and perfused regions, FIG. 24A shows the matrisome category composition by fractionation proteomics, cellular fraction, for the Ischemic region. FIG. 24B shows the matrisome category composition by fractionation proteomics, cellular fraction, for the Perfused region.

FIG. 24C shows the matrisome category composition by fractionation proteomics, soluble fraction, for the Ischemic region. FIG. 24D shows the matrisome category composition by fractionation proteomics, soluble fraction, for the Perfused region. FIG. 24E shows the matrisome category composition by fractionation proteomics, insoluble fraction, for the Ischemic region. FIG. 24F shows the matrisome category composition by fractionation proteomics, insoluble fraction, for the Perfused region.

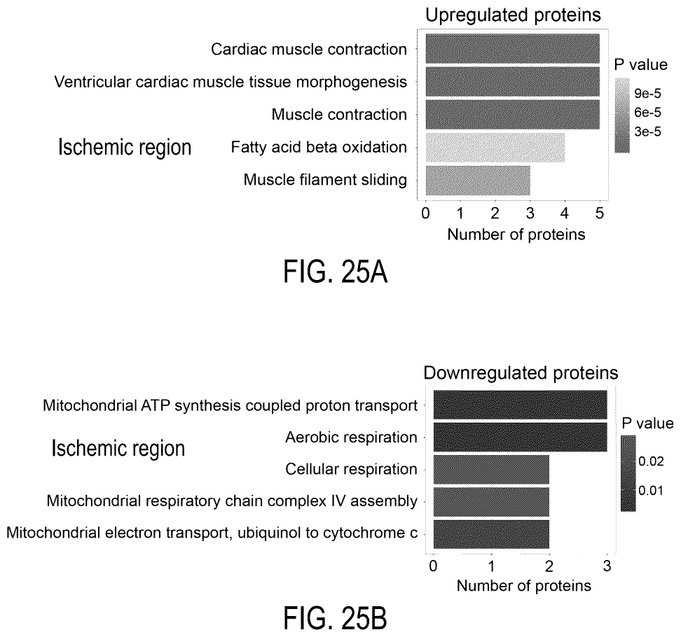

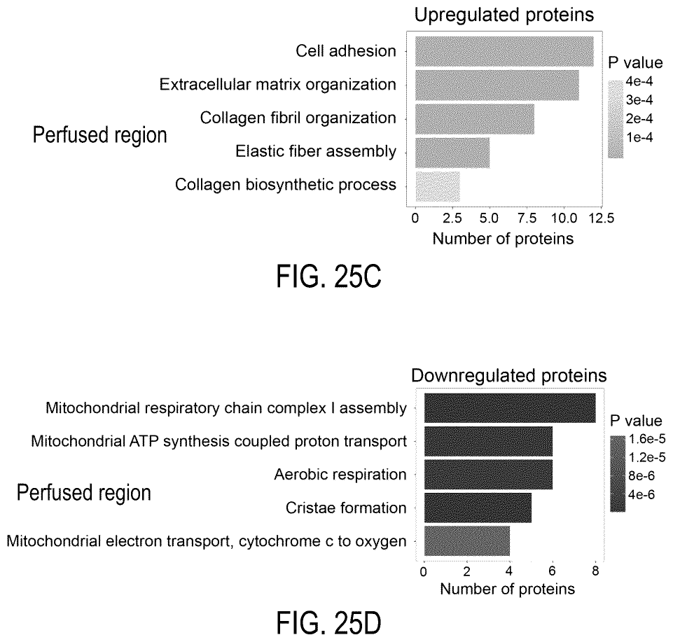

FIG. 25A shows the biological process enrichment of significantly upregulated or downregulated proteins from ischemic regions of hearts injected with ECM particles vs. saline, in the Ischemic region (upregulated). FIG. 25B shows the biological process enrichment of significantly upregulated or downregulated proteins from ischemic regions of hearts injected with ECM particles vs. saline, in the Ischemic region (downregulated).

FIG. 25C shows the biological process enrichment of significantly upregulated or downregulated proteins from perfused regions of hearts injected with ECM particles vs. saline, in the Perfused region (upregulated). FIG. 25D shows the biological process enrichment of significantly upregulated or downregulated proteins from perfused regions of hearts injected with ECM particles vs. saline, in the Perfused region (downregulated).

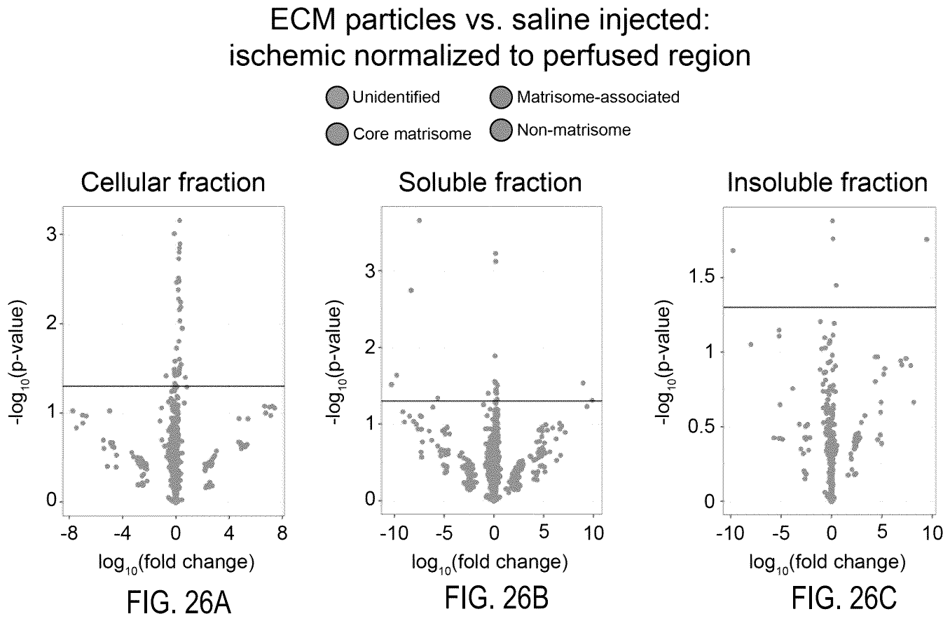

FIG. 26A shows the Cellular fraction plot for ECM particles vs. saline injected: ischemic normalized to perfused region. FIG. 26B shows the Soluble fraction plot for ECM particles vs. saline injected: ischemic normalized to perfused region. FIG. 26C shows the Insoluble fraction plot for ECM particles vs. saline injected: ischemic normalized to perfused region.

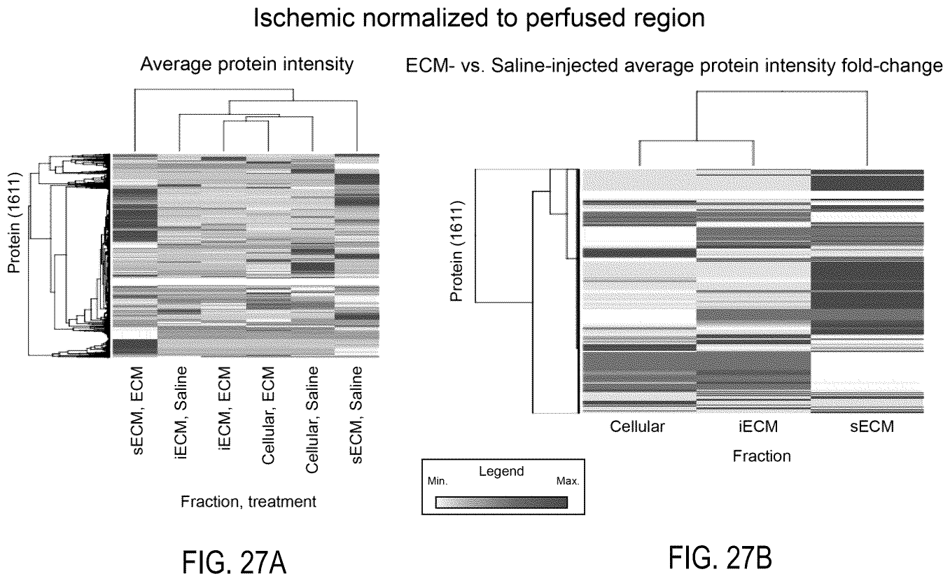

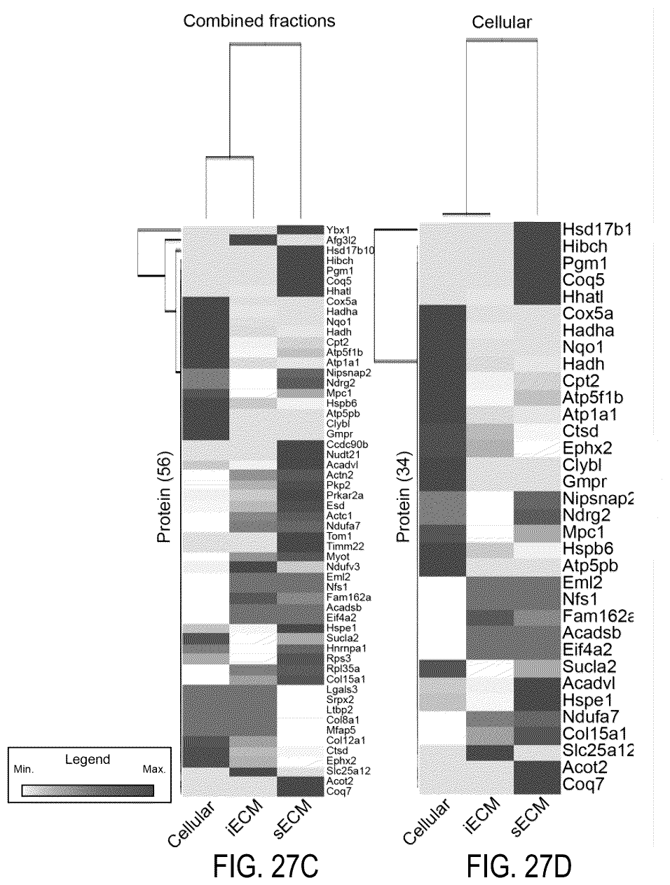

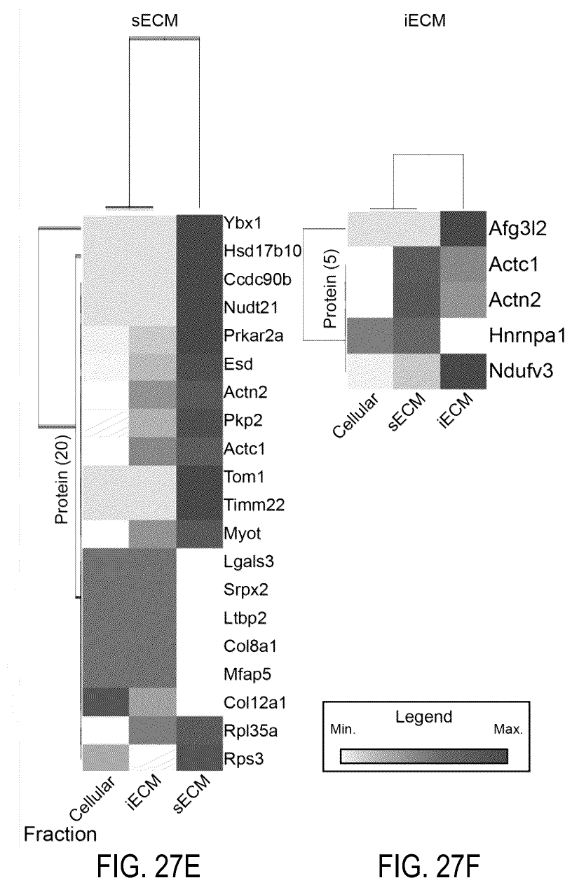

FIG. 27A shows a heat map of Ischemic normalized to perfused region, Average protein intensity. FIG. 27B shows a heat map of Ischemic normalized to perfused region, ECM-vs. Saline-injected average protein intensity fold-change.

FIG. 27C shows a heat map of Combined fractions, ECM-vs. Saline-injected average protein intensity fold-change, p-value <0.05. FIG. 27D shows a heat map of Cellular, ECM-vs. Saline-injected average protein intensity fold-change, p-value <0.05. FIG. 27E shows a heat map of SECM, ECM-vs. Saline-injected average protein intensity fold-change, p-value <0.05. FIG. 27F shows a heat map of iECM, ECM-vs. Saline-injected average protein intensity fold-change, p-value <0.05.

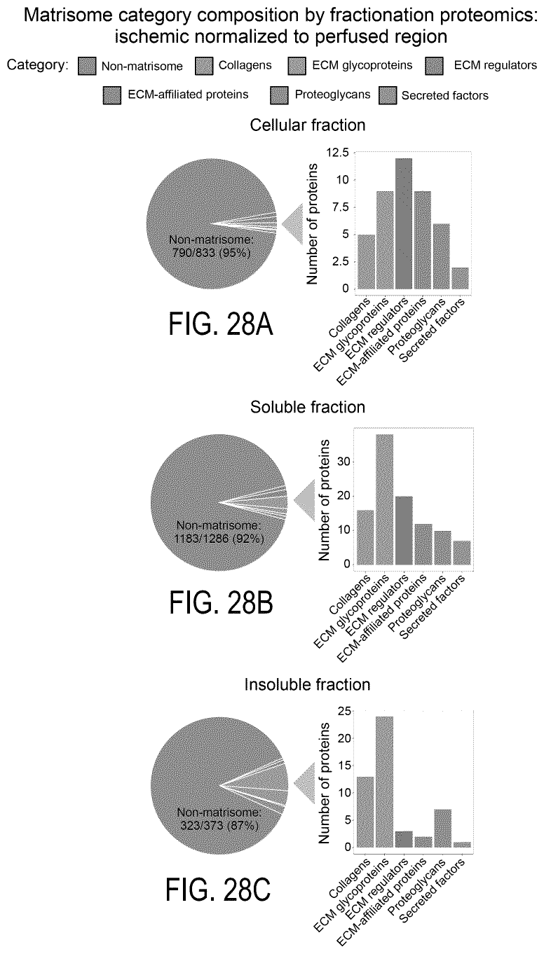

FIG. 28A shows matrisome category composition by fractionation proteomics: ischemic normalized to perfused region, for Cellular fraction. FIG. 28B shows matrisome category composition by fractionation proteomics: ischemic normalized to perfused region, for Soluble fraction. FIG. 28C shows matrisome category composition by fractionation proteomics: ischemic normalized to perfused region, for Insoluble fraction.

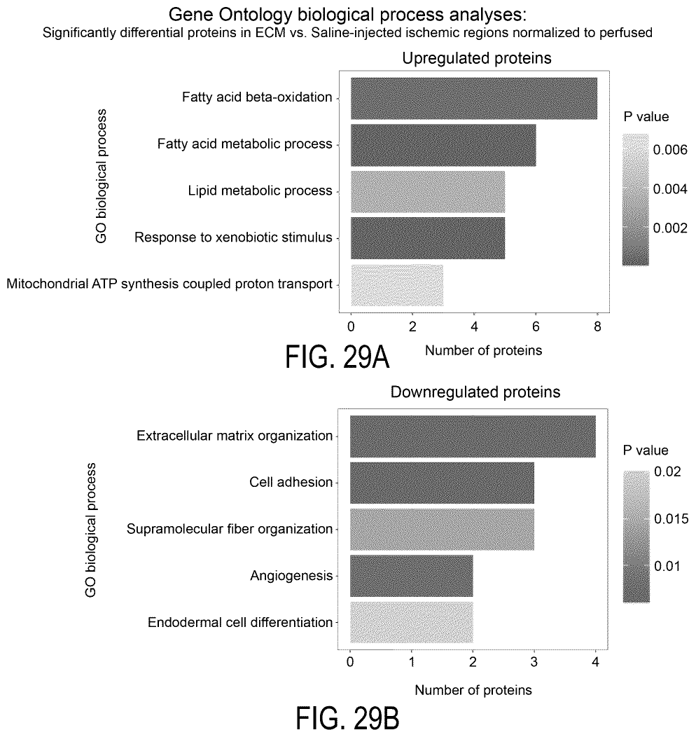

FIG. 29A shows upregulated proteins for the gene ontology biological process analyses (significantly differential proteins in the ECM vs. Saline-injected ischemic regions normalized to perfused). FIG. 29B shows downregulated proteins for the gene ontology biological process analyses (significantly differential proteins in the ECM vs. Saline-injected ischemic regions normalized to perfused). All of these data were acquired to fully enable the Invention disclosed here.

In some embodiments, the technology provides a method to alter the proteome of a diseased heart by the step of: contacting the diseased heart with extra-cellular matrix (ECM) particles produced from decellularized 3D microtissues of human mesenchymal stem cells, whereby the contacting changes the proteome of the diseased heart, restores the normal ratio of the levels of different collagens in the heart, alters the immune response in the heart, or alters metabolism and mitigates the effects of a TGF-Beta, a growth factor associated with fibrosis. The treatment of heart failure and other indications where scarring and fibrosis are problematic are major unmet medical needs. The extra-cellular matrix (ECM) particles provide a new regenerative therapy to treat diseases whose underlying pathology is multi-factorial.

### REFERENCES

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize, or be able to ascertain using no more than routine experimentation, many equivalents to the specific embodiments described herein. Such equivalents are intended to be encompassed by the following claims.
ⁱBlaheta, R. A., et al. (1998). “Development of an ultrasensitive in vitro assay to monitor growth of primary cell cultures with reduced mitotic activity.” J. Immunol. Methods 211(1-2): 159-169.
ⁱⁱCissell, D. D., et al. (2017). “A Modified Hydroxyproline Assay Based on Hydrochloric Acid in Ehrlich's Solution Accurately Measures Tissue Collagen Content.” Tissue Eng. Part C Methods 23(4): 243-250.
ⁱⁱⁱFarndale, R. W., et al. (1982). “A direct spectrophotometric microassay for sulfated glycosaminoglycans in cartilage cultures.” Connect. Tissue Res. 9(4): 247-248.
^{iv}Whitley, C. B., et al. (1989). “Diagnostic test for mucopolysaccharidosis. I. Direct method for quantifying excessive urinary glycosaminoglycan excretion.” Clin. Chem. 35(3): 374-379.
^{v}Naba, A., et al. (2015). “Enrichment of Extracellular Matrix Proteins from Tissues and Digestion into Peptides for Mass Spectrometry Analysis.” J. Vis. Exp. 101: e53057.
^{vi}Schwanhäusser, B., et al. (2011). “Global quantification of mammalian gene expression control.” Nature, 473(7347): 337-342.
^{vii}Özbek, S., et al. (2010). “The evolution of extracellular matrix.” Mol. Biol. Cell 21:4300-4305.
^{viii}Naba, A. et al. (2016). “The extracellular matrix: tools and insights for the ‘omics’ era.” Matrix Biol. 49, 10-24.
^{ix}International Publication No. WO 2022/140530, Ip, B. C. and Morgan, J. R. “DECELLULARIZED MAMMALIAN EXTRACELLULAR MATRIX MORSELS, METHODS MAKING AND METHODS OF USING SAME.”
^{x}Verrecchia, F. & Mauviel, A. (2002). “Transforming growth factor-beta signaling through the Smad pathway: role in extracellular matrix gene expression and regulation.” J. Invest. Dermatol. 118(2): 211-215.
^{xi}Horiguchi, M., et al. (2012). “Matrix control of transforming growth factor-β function.” J. Biochem. 152(4): 321-329.

We claim:

1. A method of altering the proteome of a diseased heart, comprising:
contacting the diseased heart with extra-cellular matrix (ECM) particles produced from decellularized 3D microtissues,
wherein the contacting alters the proteome of the diseased heart.

2. The method of claim 1, wherein the contacting restores a normal ratio of levels of different collagens in the heart.

3. The method of claim 1, wherein the contacting alters an immune response in the heart.

4. The method of claim 1, wherein the contacting alters metabolism in the heart.

5. The method of claim 1, wherein the contacting mitigates effects of TGF-Beta in the heart, wherein TGF-Beta is a growth factor associated with fibrosis.

6. The method of claim 1, wherein the 3D microtissues are derived from human mesenchymal stem cells.

7. A method of treating a diseased heart, comprising:
administering to the diseased heart extra-cellular matrix (ECM) particles produced from decellularized 3D microtissues,
wherein the administering treats the diseased heart by altering the proteome of the diseased heart.

8. The method of claim 7, wherein the administering restores a normal ratio of levels of different collagens in the heart.

9. The method of claim 7, wherein the administering alters an immune response in the heart.

10. The method of claim 7, wherein the administering alters metabolism in the heart.

11. The method of claim 7, wherein the administering mitigates effects of TGF-Beta in the heart, wherein TGF-Beta is a growth factor associated with fibrosis.

12. The method of claim 7, wherein the 3D microtissues are derived from human mesenchymal stem cells.

13. Extra-cellular matrix (ECM) particles for altering the proteome of a diseased heart, wherein the ECM particles are produced from decellularized 3D microtissues and treated to improve one or more of crosslinking, stability, injectability, or compatibility with a patient.

14. The ECM particles of claim 13, wherein the ECM particles, when contacted with the diseased heart, restore a normal ratio of levels of different collagens in the heart.

15. The ECM particles of claim 13, wherein the ECM particles, when contacted with the diseased heart, alter an immune response in the heart.

16. The ECM particles of claim 13, wherein the ECM particles, when contacted with the diseased heart, alter metabolism in the heart.

17. The ECM particles of claim 13, wherein the ECM particles, when contacted with the diseased heart, mitigate effects of TGF-Beta in the heart, wherein TGF-Beta is a growth factor associated with fibrosis.

18. The ECM particles of claim 13, wherein the 3D microtissues are derived from human mesenchymal stem cells.

19. A composition for treating a diseased heart, comprising:
extra-cellular matrix (ECM) particles produced from decellularized 3D microtissues and treated to improve one or more of crosslinking, stability, injectability, or compatibility with a patient; wherein the composition,
when administered to the diseased heart, treats the diseased heart by altering the proteome of the diseased heart.

20. The composition of claim 19, wherein the 3D microtissues are derived from human mesenchymal stem cells.
